(19) United States
(12) Patent Application Publication
(10) Pub. No.: US 2026/0355367 A1
(43) Pub. Date: Nov. 12, 2026

(54) RADIONUCLIDE COMPOSITION AND METHOD OF USING SAME FOR DETECTION OF TUMOR CELLS

(71) Applicant: The Research Foundation for The State University of New York, Albany, NY (US)

(72) Inventors: Jacob Houghton, Stony Brook, NY (US); Vilma Jallinoja, Stony Brook, NY (US)

(73) Assignee: The Research Foundation for The State University of New York, Albany, NY (US)

(21) Appl. No.: 19/108,598

(22) PCT Filed: Sep. 8, 2023
(86) PCT No.: PCT/US2023/073725

§ 371 Date: Mar. 4, 2025

Related U.S. Application Data
(60) Provisional application No. 63/375,159, filed on Sep. 9, 2022.
(60) Provisional application No. 63/481,517, filed on Jan. 25, 2023.

(51) Int. Cl. A61K 51/10

(52) U.S. Cl. CPC A61K 51/1093

(57) ABSTRACT

The present invention provides a compound having the structure:

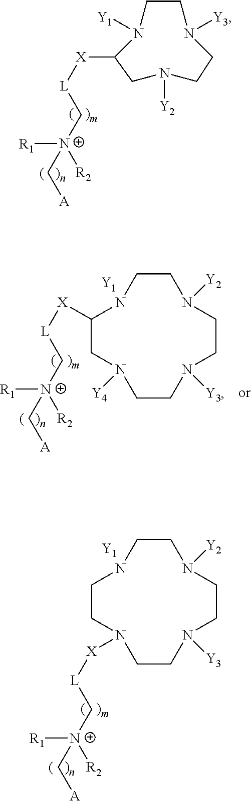

or salt or ester thereof.

This application claims priority of U.S. Provisional Application No. 62/481,517, filed Jan. 25, 2023 and U.S. Provisional Application No. 63/375,159, filed Sep. 9, 2022, the contents of which are hereby incorporated by reference.

### STATEMENT REGARDING FEDERALLY SPONSORED RESEARCH OR DEVELOPMENT

This invention was made with government support under EB027982 awarded by National Institutes of Health. The government has certain rights in the invention.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### BACKGROUND OF THE INVENTION

Pretargeted positron emission tomography (PET) provides quantitative, non-invasive whole-body in vivo profile of macromolecules with an overall lower total body radiation dose compared to directly radiolabeled macromolecules (Altai, 2017 and Jallinoja, 2021). Pretargeting is a two-step strategy that involves the administration of a target binding macromolecule which accumulates at the target site over several days while the unbound macromolecule excretes from non-target tissue. In the second step, a bioorthogonal small molecule radioligand is administered. (FIG. 1) The low molecular weight of the radioligand allows its target accumulation and excretion to occur faster than that of the initial macromolecule. The present invention harnesses a host-guest complex formation as the specific pretargeting interaction between the macromolecule and the radioligand. It was hypothesized that due to the high in vivo stability, modularity and low immunogenicity, the chosen host-guest pair, cucurbit[7]uril-adamantane (CB7-Adma, Kₐ~10¹⁴ M⁻¹) makes an ideal interaction pair for pretargeted PET (Assaf K I, 2015, Shetty D, 2015 and Wanka L, 2013). The strong complex between the two molecules forms when the Adma guest with an adjacent positively charged moiety binds to the carbonyl framed cavity of the macrocyclic CB7 host molecule via multiple van der Waals and ion-dipole interactions. So far, the medical imaging applications utilizing host-guest chemistry have been limited to pre-formed host-guest complexes to increase stability and/or sensitivity of imaging agents (Zhao, 2022, Sembo-Backonly, 2021 and Wu 2021). In nuclear medicine, the high affinity non-covalent binding between CB7 and Adma molecules has remained minimally explored (Strebl M G, 2018).

Three copper-64-labeled Adma guest molecules [⁶⁴Cu]Cu-NOTA-Adma (1), [⁶⁴Cu]Cu-NOTA-PEG₃-Adma (2) and [⁶⁴Cu]Cu-NOTA-PEG₇-Adma (3) were synthesized and characterized. (FIG. 2A) The in vivo profile of the ligands for pretargeting was evaluated using a CB7-modified carcinoembryonic antigen (CEA) targeting humanized full-length antibody (CB7-M5A) as the secondary pretargeting agent. To study the potential of the platform, two pretargeting lag time schedules, 72 and 144 h, were investigated. The pretargeting studies were performed in BxPC3 (CEA+) and MIAPaCa-2 (CEA-) human pancreatic cancer mouse xenografts (Girgis 2011, Yunis 1977 and Tan 1986).

The biodistribution and dosimetry of the pretargeted Adma-radioligand was compared to that of a directly zirconium-89-labeled M5A. The high stability, mutual high affinity and human compatibility of the proposed CB7-Adma pretargeting agents provide a great basis for a pretargeting platform that is widely applicable.

### BRIEF SUMMARY OF THE INVENTION

The present invention provides a compound having the structure:

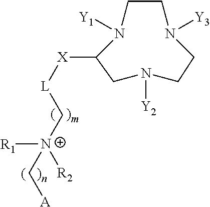

wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
wherein each occurrence of R₄ is independently, —H, —OH, —NH₂, halogen, alkyl, —O-alkyl, —NH-alkyl, —CHF₂, —CF₃, —OCHF₂, —OCF₃, alkenyl, alkynyl, alkyl-aryl, alkyl-heteroaryl, aryl, heteroaryl, alkyl-CF₃, or —Si(alkyl)₃, and
   wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

The present invention provides a compound having the structure:

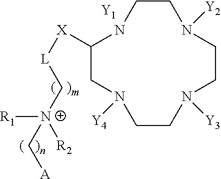

wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
wherein each occurrence of R₄ is independently, —H, —OH, —NH₂, halogen, alkyl, —O-alkyl, —NH-alkyl, —CHF₂, —CF₃, —OCHF₂, —OCF₃, alkenyl, alkynyl, alkyl-aryl, alkyl-heteroaryl, aryl, heteroaryl, alkyl-CF₃, or —Si(alkyl)₃, and
   wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

The present invention provides a compound having the structure:

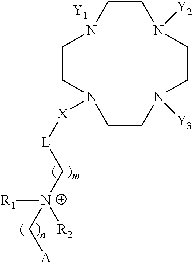

wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
wherein each occurrence of R₄ is independently, —H, alkyl, alkenyl, alkynyl, alkyl-aryl, alkyl-heteroaryl, aryl, heteroaryl, alkyl-CF₃, or —Si(alkyl)₃;
   wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

### BRIEF DESCRIPTION OF THE DRAWINGS

FIGS. 1A and 1B. An illustration of the two-step cucurbit[7]uril-adamantane pretargeting approach.

FIG. 2. The chemical structure of the [⁶⁴Cu]Cu-NOTA-Adma (1), [⁶⁴Cu]Cu-NOTA-PEG₃-Adma (2) and [⁶⁴Cu]Cu-NOTA-PEG₇-Adma (3) and their respective distribution coefficient (log D) and blood half-lives (A). The in vitro stability of 1-3 in phosphate buffer saline (PBS pH=7.4) and in bovine plasma at 37 °C and their plasma protein binding. (B)

FIG. 3. The in vivo biodistribution of pretargeted [⁶⁴Cu]Cu-NOTA-Adma; 1, [⁶⁴Cu]Cu-NOTA-PEG₃-Adma; 2 and [⁶⁴Cu]Cu-NOTA-PEG₇-Adma; 3 in BxPC3 tumor bearing nude mice. % ID/g; percentage of injected dose & ns; not significant & *p<0.05

FIG. 4. Maximum intensity projection PET images of the pretargeted [⁶⁴Cu]Cu-NOTA-Adma; 1, [⁶⁴Cu]Cu-NOTA-PEG₃-Adma; 2 and [⁶⁴Cu]Cu-NOTA-PEG₇-Adma; 3 in BxPC3 tumor bearing nude mice at 4, 8 and 24 h post radioligand injection with a 72 h lag time pretargeting schedule. The location of the tumor on the right shoulder is highlighted with a red circle.

FIG. 5. In vivo profile of [⁸⁹Zr]Zr-DFO-M5A at 72 h post injection and pretargeted 2 at 24 h post injection in BxPC3 and MIAPaCa-2 tumor bearing female nude mice. Maximum intensity projection PET images of [⁸⁹Zr]Zr-DFO-M5A (A), pretargeted 2 (B) and the in vivo biodistribution of [⁸⁹Zr]Zr-DFO-M5A (C) and pretargeted 2 (D). The mice administered with 2 were injected with CB7-M5A 72 h prior. The location of the tumor on the right shoulder is highlighted with a red circle.

FIG. 6. The reaction scheme for the synthesis of 4 and 5. I. triethylamine, methanol; II. sodium triacetoxyborohydride; III. iodomethane, sodium hydroxide; IV. trifluoroacetic acid, dichloromethane; V. p-SCN-Bn-NOTA×3 HCl, N,N-diisopropylethylamine and dimethyl sulfoxide.

FIG. 7. The reaction scheme for the synthesis of 6 and 7. I. triethylamine, methanol; II. sodium triacetoxyborohydride; III. iodomethane, sodium hydroxide, triethylamine; IV. trifluoroacetic acid, dichloromethane; V. p-SCN-Bn-NOTA×3 HCl, N,N-diisopropylethylamine, dimethyl sulfoxide.

FIG. 8. The reaction scheme for the synthesis of 8 and 9. I. triethylamine, methanol; II. sodium triacetoxyborohydride; III. iodomethane, sodium hydroxide; IV. trifluoroacetic acid, dichloromethane; V. p-SCN-Bn-NOTA×3 HCl, N,N-diisopropylethylamine, dimethyl sulfoxide.

FIG. 9. UV-Vis HPLC chromatograph of NOTA-Adma (5) monitoring absorption of wavelength 254 nm.

FIG. 10. UV-Vis HPLC chromatograph of NOTA-PEG₃-Adma (7) monitoring absorption of wavelength 254 nm.

FIG. 11. UV-Vis HPLC chromatograph of NOTA-PEG₇-Adma (9) monitoring absorption of wavelength 254 nm.

FIG. 12 Radio-HPLC chromatograph of [⁶⁴Cu]Cu-NOTA-Adma (1).

FIG. 13 Radio-HPLC chromatograph of [⁶⁴Cu]Cu-NOTA-PEG₃-Adma (2).

FIG. 14. Radio-HPLC chromatograph of [⁶⁴Cu]Cu-NOTA-PEG₇-Adma (3).

FIG. 15. FPLC chromatograph of the non-modified M5A (A), CB7-M5A (B) and DFO-M5A (C) with detection of absorption of 280 nm.

FIG. 16. The percentage of internalized and membrane bound [⁶⁴Cu]CuCl₂ and [⁶⁴Cu]Cu-NOTA-PEG₃-Adma (2) of total added activity over 6 hours in BxPC3 cells.

FIG. 17. The two-phase decay curves are based on the % ID/g of the blood samples as a function of time. [⁶⁴Cu]Cu-NOTA-Adma (A), [⁶⁴Cu]Cu-NOTA-PEG₃-Adma (B) and [⁶⁴Cu]Cu-NOTA-PEG₇-Adma (C).

FIG. 18. Western Blotting of carcinoembryonic antigen (CEA) in BxPC3 and MIAPaCa-2 cell lysates. Two CEA-targeting antibodies were used, hT84.66-M5A and H.426.3.

FIG. 19. Maximum intensity projection PET images of the pretargeted [⁶⁴Cu]Cu-NOTA-PEG₃-Adma; 2 in BxPC3 tumor bearing nude mice at 4, 8 and 24 h post radioligand injection. 144 h prior to the radioligand injection mice were administered with CB7-M5A. The location of the tumor on the right shoulder is highlighted with a red circle.

### DETAILED DESCRIPTION OF THE INVENTION

The present invention provides a compound having the structure:

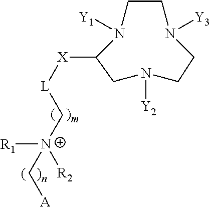

wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine,
or salt or ester thereof.

The present invention provides a compound having the structure:

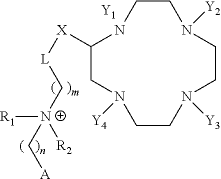

wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

The present invention provides a compound having the structure:

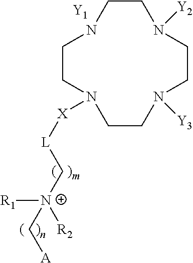

wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, or salt or ester thereof.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, n and m are each independently 0, 1, 2, 3, 4, 5, or 6.

In some embodiments, n and m are each independently 1, 2, or 3.

In some embodiments, n and m are each independently 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, R₁ and R₂ are ethyl.

In some embodiments, X is alkyl-aryl-thiourea, alkyl-heteroaryl-thiourea, alkyl-cycloalkyl-thiourea, alkenyl-aryl-thiourea, or alkenyl-heteroaryl-thiourea.

In some embodiments, X is alkyl-aryl-thiourea, alkyl-heteroaryl-thiourea, or alkyl-cycloalkyl-thiourea.

In some embodiments, X is alkyl-aryl-thiourea, or alkyl-heteroaryl-thiourea.

In some embodiments, X is alkyl-aryl-thiourea.

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

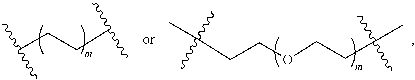

wherein m is 1, 2, 3, 4, 5, 6, 7, 8, 9, or 10.

In some embodiments of chemical linker L, m is 1, 2, 3, 4, 5, 6, or 7.

In some embodiments of chemical linker L, m is 1, 3, or 7.

In some embodiments of chemical linker L, m is 3.

In some embodiments, the chemical linker L has the following structure:

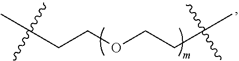

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments of chemical linker L, m is 3.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, amide.

In some embodiments, each occurrence of R₄ is independently, —OH, —NH₂, alkyl, —O-alkyl, —NH— alkyl.

In some embodiments, R₄ is —OH.

In some embodiments, R₄ is —NH₂.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃, Y₄ are each independently alkyl-CO₂H, or alkyl-CO₂NH₂, or alkyl-N(alkyl-CO₂H)₂.

In some embodiments, at least one of Y₁, Y₂, Y₃, Y₄ is alkyl-CO₂H.

In some embodiments, at least one of Y₁, Y₂, Y₃, Y₄ is alkyl-CO₂NH₂.

In some embodiments, at least one of Y₁, Y₂, Y₃, Y₄ is alkyl-N(alkyl-CO₂H)₂.

In some embodiments, at least one of Y₁, Y₂, Y₃, Y₄ is —CH₂—CO₂H.

In some embodiments, at least one of Y₁, Y₂, Y₃, Y₄ is —CH₂—CO₂NH₂.

In some embodiments, at least one of Y₁, Y₂, Y₃, Y₄ is —CH₂—N(alkyl-CO₂H)₂

In some embodiments, at least two of Y₁, Y₂, Y₃, Y₄ are the same.

In some embodiments, at least three of Y₁, Y₂, Y₃, Y₄ are the same.

In some embodiments, Y₁, Y₂, Y₃, Y₄ are the same.

In some embodiments, at least one of Y₁, Y₂, Y₃ and Y₄ is H.

In some embodiments, none of Y₁, Y₂, Y₃ and Y₄ are H.

In some embodiments, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃ are each independently alkyl-CO₂H, or alkyl-CO₂NH₂, or alkyl-N(alkyl-CO₂H)₂.

In some embodiments, at least one of Y₁, Y₂, Y₃ is alkyl-CO₂H.

In some embodiments, at least one of Y₁, Y₂, Y₃ is alkyl-CO₂NH₂.

In some embodiments, at least one of Y₁, Y₂, Y₃ is alkyl-N(alkyl-CO₂H)₂.

In some embodiments, at least one of Y₁, Y₂, Y₃ is —CH₂—CO₂H.

In some embodiments, at least one of Y₁, Y₂, Y₃ is —CH₂—CO₂NH₂.

In some embodiments, at least one of Y₁, Y₂, Y₃ is —CH₂—N(alkyl-CO₂H)₂

In some embodiments, at least two of Y₁, Y₂ and Y₃ are the same.

In some embodiments, Y₁, Y₂ and Y₃ are the same.

In some embodiments, at least one of Y₁, Y₂, and Y₃ is H.

In some embodiments, none of Y₁, Y₂, and Y₃ are H.

In some embodiments, Y₁, Y₂, Y₃, Y₄ are each independently —H,

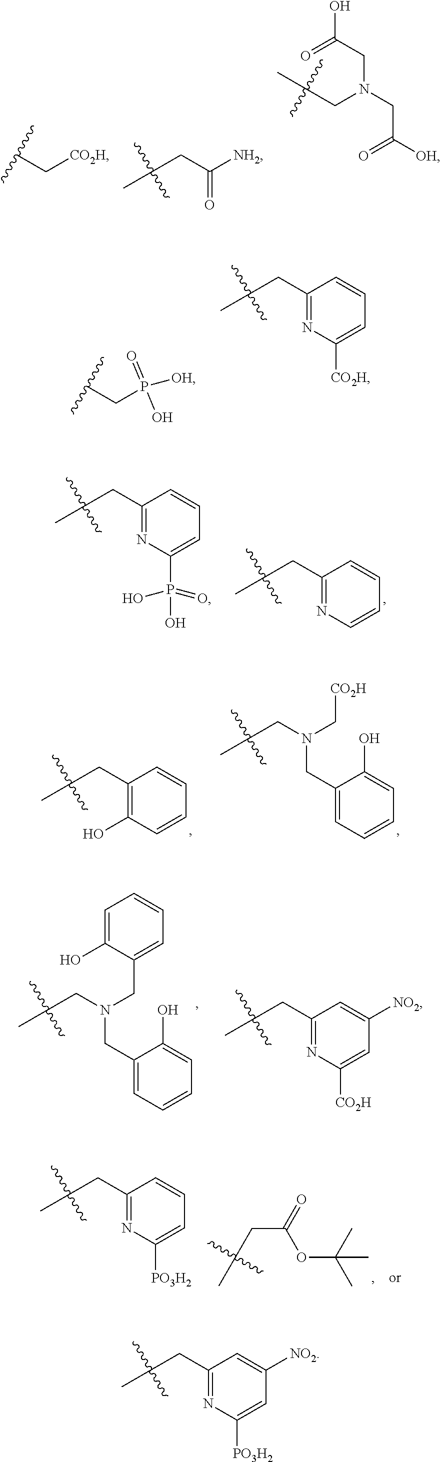

In some embodiments, Y₁, Y₂, Y₃, Y₄ are each independently

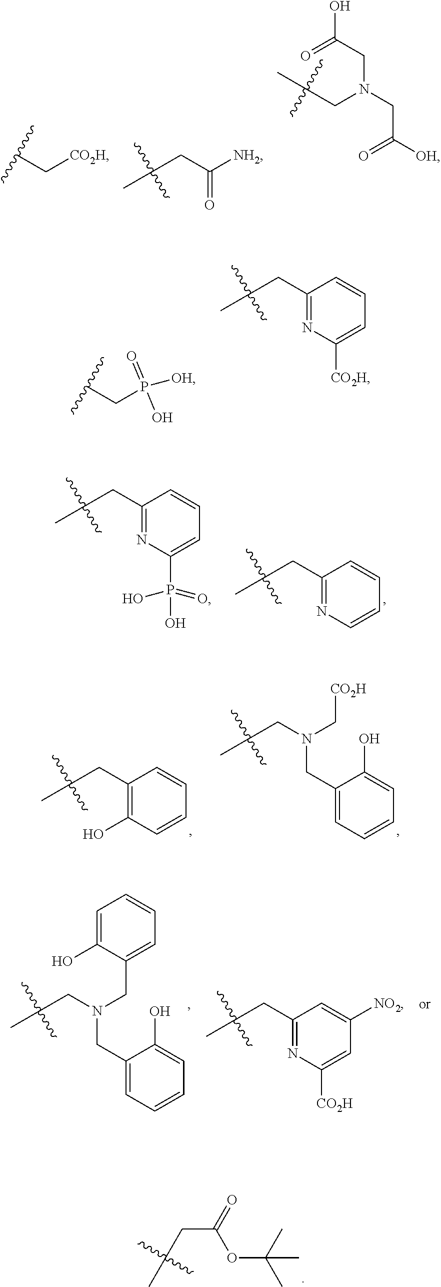

In some embodiments, Y₁, Y₂, Y₃, Y₄ are each independently

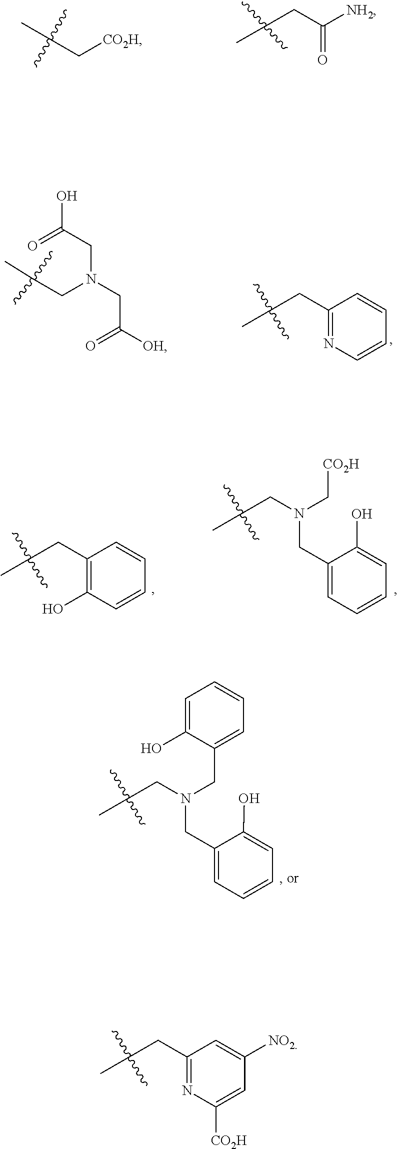

In some embodiments, Y₁, Y₂, Y₃, Y₄ are each independently

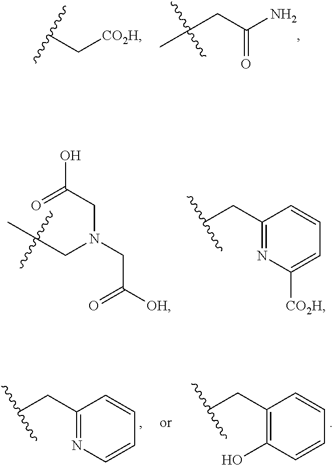 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]

In some embodiments, Y₁, Y₂, Y₃, Y₄ are each independently

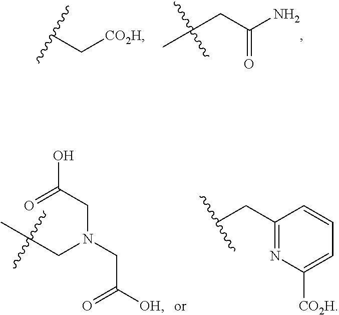 [SMILES: *CC(=O)O ; *CC(N)=O ; *CN(CC(=O)O)CC(=O)O ; *Cc1cccc(C(=O)O)n1]

In some embodiments, Y₁, and Y₃ is

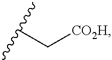 [SMILES: *CC(=O)O]

Y₂ and/or Y₄ is

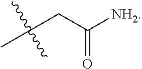 [SMILES: *CC(N)=O]

In some embodiments, the guest molecule A is substituted or unsubstituted adamantane, diamantane, 4,9-diamino diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, diamantane, 4,9-diamino diamantane or ferrocene.

In some embodiments, the guest molecule A is substituted or unsubstituted adamantane or diamantane.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, the guest molecule A is substituted with halogen, alkyl, —O-(alkyl), —N-(alkyl).

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

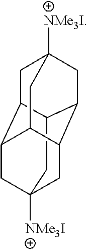

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments, the substituted ferrocene is substituted with C₁-C₆ alkyl, -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

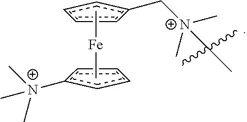

In some embodiments, the present invention provides a compound having the structure:

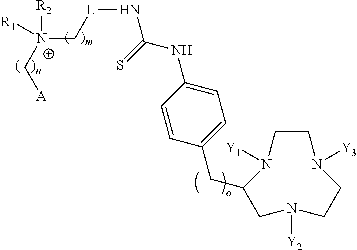

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R, alkylaryl-NH—CO₂R, alkylaryl-CO₂R, alkylheteroaryl-CO₂R, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; or salt or ester thereof.

In some embodiments, the present invention provides a compound having the structure:

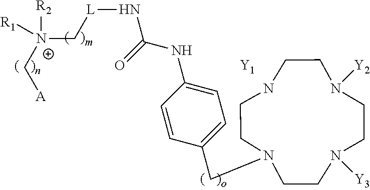

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, the compound is other than

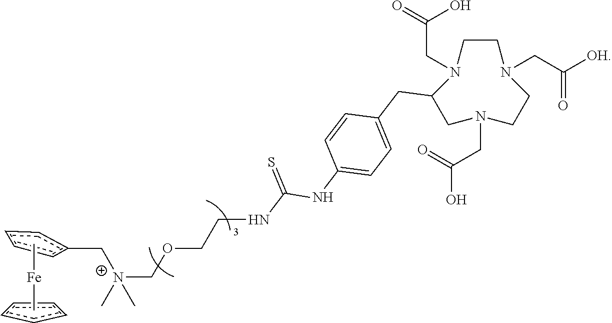

In some embodiments, n and m are each independently 0, 1, 2, 3, 4, 5, or 6.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, o is 0, 1, 2, 3, 4, 5, or 6.

In some embodiments, o is 0, 1, 2, or 3.

In some embodiments, o is 1 or 2.

In some embodiments, o is 1.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, R₁ and R₂ are ethyl.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, amide.

In some embodiments, each occurrence of R₄ is independently, —OH, —NH₂, alkyl, —O-alkyl, —NH— alkyl.

In some embodiments, R₄ is —OH.

In some embodiments, R₄ is —NH₂.

In some embodiments, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃ are each independently alkyl-CO₂H, or alkyl-CO₂NH₂, or alkyl-N(alkyl-CO₂H)₂.

In some embodiments, at least one of Y₁, Y₂, Y₃ is alkyl-CO₂H.

In some embodiments, at least one of Y₁, Y₂, Y₃ is alkyl-CO₂NH₂.

In some embodiments, at least one of Y₁, Y₂, Y₃ is alkyl-N(alkyl-CO₂H)₂.

In some embodiments, at least one of Y₁, Y₂, Y₃ is —CH₂—CO₂H.

In some embodiments, at least one of Y₁, Y₂, Y₃ is —CH₂—CO₂NH₂.

In some embodiments, at least one of Y₁, Y₂, Y₃ is —CH₂—N(alkyl-CO₂H)₂

In some embodiments, at least two of Y₁, Y₂ and Y₃ are the same.

In some embodiments, Y₁, Y₂ and Y₃ are the same.

In some embodiments, at least one of Y₁, Y₂, Y₃ and Y₄ is H.

In some embodiments, none of Y₁, Y₂, Y₃ and Y₄ are H.

In some embodiments, Y₁, Y₂, Y₃, Y₄ are each independently —H,

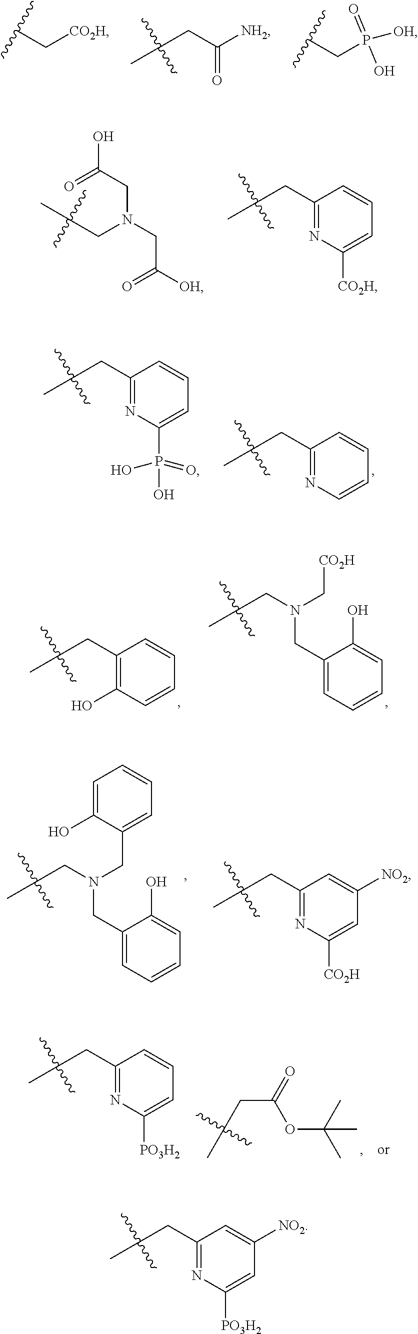

In some embodiments, Y₁, Y₂, Y₃, Y₄ are each independently

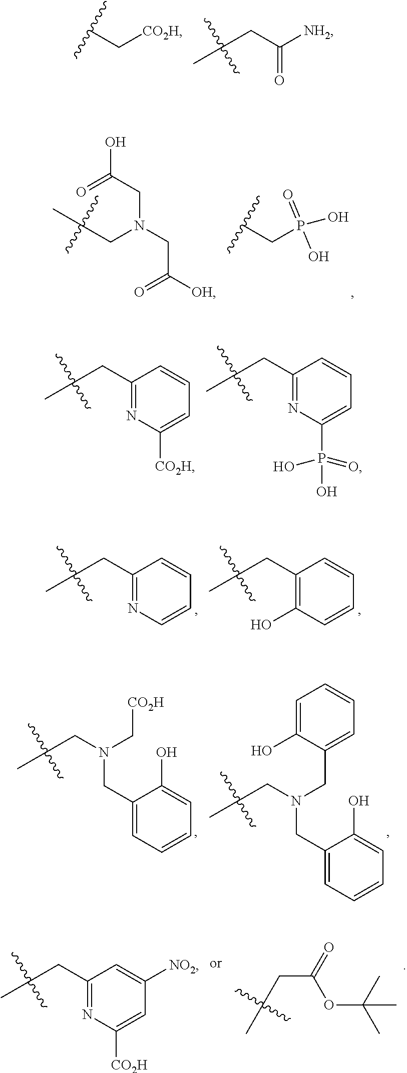

In some embodiments, Y₁, Y₂, Y₃, Y₄ are each independently

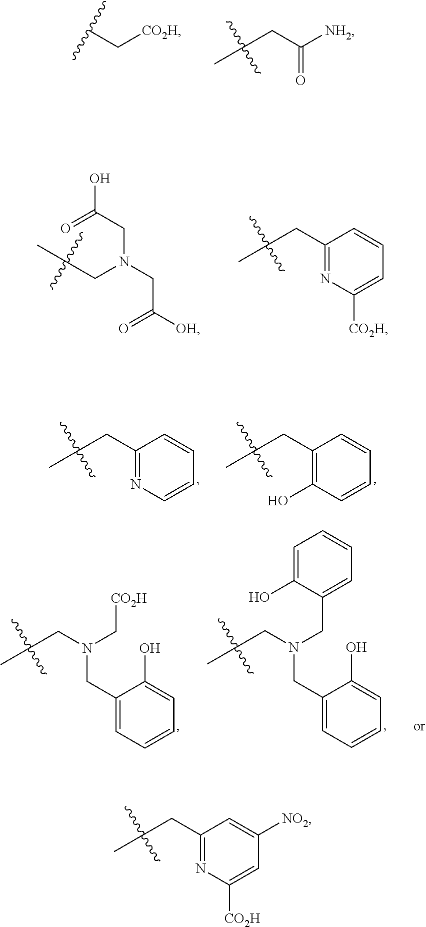

In some embodiments, Y₁, Y₂, Y₃, Y₄ are each independently

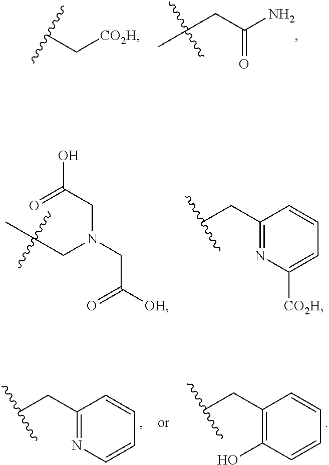 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]

In some embodiments, Y₁, Y₂, Y₃, Y₄ are each independently

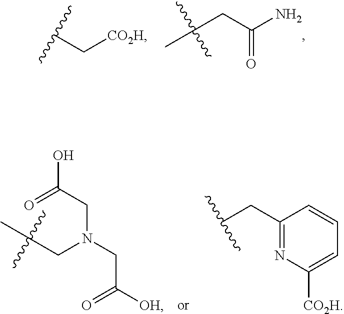 [SMILES: *CC(=O)O ; *CC(N)=O ; *CN(CC(=O)O)CC(=O)O ; *Cc1cccc(C(=O)O)n1]

In some embodiments, Y₁ and Y₃ is

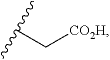 [SMILES: *CC(=O)O]

Y₂ and/or Y₄ is

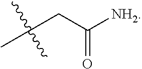 [SMILES: *CC(N)=O]

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

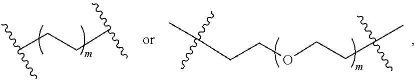

wherein m is 1, 2, 3, 4, 5, 6, 7, 8, 9, or 10; preferably m is 1, 2, 3, 4, 5, 6, or 7; more preferably, m is 1, 3, or 7.

In some embodiments, the chemical linker L has the following structure:

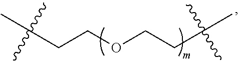

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments of chemical linker L, m is 3.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, amide.

In some embodiments, each occurrence of R₄ is independently, —OH, —NH₂, alkyl, —O-alkyl, —NH— alkyl.

In some embodiments, R₄ is —OH.

In some embodiments, R₄ is —NH₂.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, the guest molecule A is substituted or unsubstituted adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is substituted or unsubstituted adamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, diamantane, 4,9-diamino diamantane or ferrocene.

In some embodiments, the guest molecule A is substituted or unsubstituted adamantane or diamantane.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, the guest molecule A is substituted with halogen, alkyl, —O-(alkyl), —N-(alkyl).

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

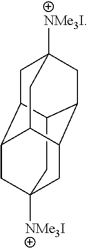

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments, the substituted ferrocene is substituted with C₁-C₆ alkyl, -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

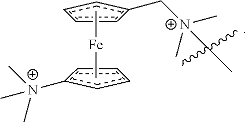

In some embodiments, the present invention provides a compound having the structure:

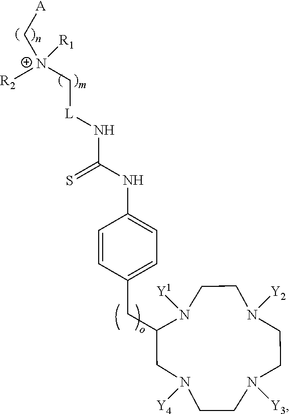

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, n and m are each independently 0, 1, 2, 3, 4, 5, or 6.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, o is 0, 1, 2, 3, 4, 5, or 6.

In some embodiments, o is 0, 1, 2, or 3.

In some embodiments, o is 1 or 2.

In some embodiments, o is 1.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH-alkyl, amide, aryl, heteroaryl, or alkyl-CF₃.

In some embodiments, each occurrence of R₄ is independently, —OH, —NH₂, alkyl, —O-alkyl, —NH-alkyl, or amide.

In some embodiments, each occurrence of R₄ is independently, —OH, —NH₂, alkyl, —O-alkyl, - or NH-alkyl.

In some embodiments, R₄ is —OH.

In some embodiments, R₄ is —NH₂.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃, Y₄ are each independently alkyl-CO₂H, or alkyl-CO₂NH₂, or alkyl-N(alkyl-CO₂H)₂.

In some embodiments, at least one of Y₁, Y₂, Y₃, Y₄ is alkyl-CO₂H.

In some embodiments, at least one of Y₁, Y₂, Y₃, Y₄ is alkyl-CO₂NH₂.

In some embodiments, at least one of Y₁, Y₂, Y₃, Y₄ is alkyl-N(alkyl-CO₂H)₂.

In some embodiments, at least one of Y₁, Y₂, Y₃, Y₄ is —CH₂—CO₂H.

In some embodiments, at least one of Y₁, Y₂, Y₃, Y₄ is —CH₂—CO₂NH₂.

In some embodiments, at least one of Y₁, Y₂, Y₃, Y₄ is —CH₂—N(alkyl-CO₂H)₂

In some embodiments, at least two of Y₁, Y₂ and Y₃, Y₄ are the same.

In some embodiments, at least three of Y₁, Y₂, Y₃ and Y₄ are the same.

In some embodiments, at least one of Y₁, Y₂, Y₃ and Y₄ is H.

In some embodiments, none of Y₁, Y₂, Y₃ and Y₄ are H.

In some embodiments, Y₁, Y₂ Y₃, and Y₄ are the same.

In some embodiments, at least one of Y₁, Y₂, Y₃ and Y₄ is H.

In some embodiments, none of Y₁, Y₂, Y₃ and Y₄ are H.

In some embodiments, Y₁, Y₂, Y₃, Y₄ are each independently —H,

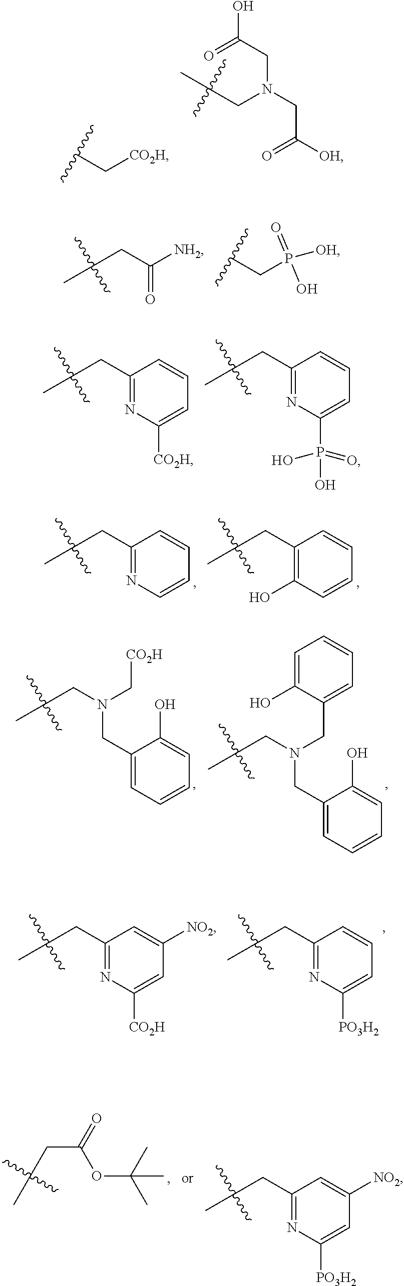

In some embodiments, Y₁, Y₂, Y₃, Y₄ are each independently

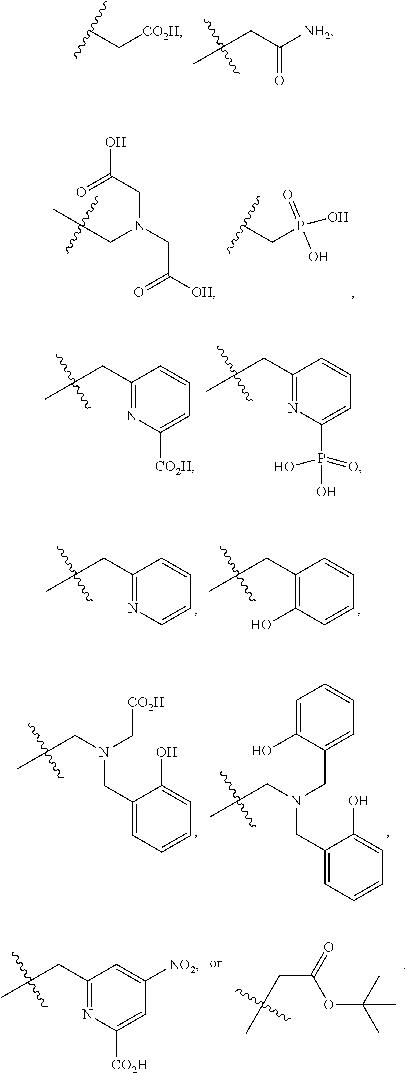

In some embodiments, Y₁, Y₂, Y₃, Y₄ are each independently

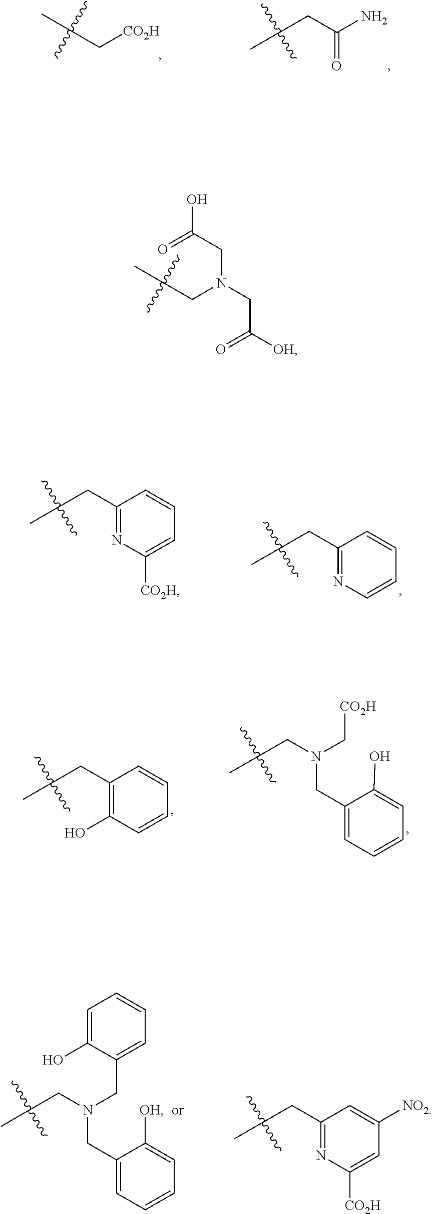

In some embodiments, Y₁, Y₂, Y₃, Y₄ are each independently

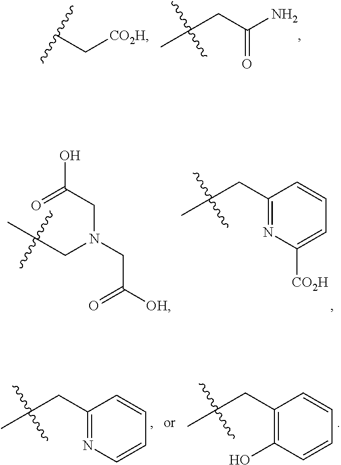 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]

In some embodiments, Y₁, Y₂, Y₃, Y₄ are each independently

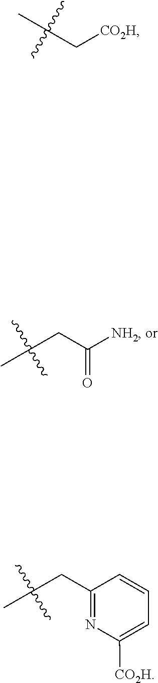

In some embodiments, Y₁ and Y₃ is

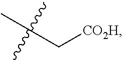 [SMILES: *CC(=O)O]

Y₂ and/or Y₄ is

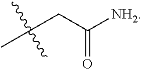 [SMILES: *CC(N)=O]

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

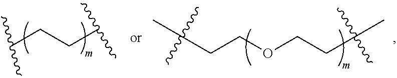

wherein m is 1, 2, 3, 4, 5, 6, 7, 8, 9, or 10; preferably m is 1, 2, 3, 4, 5, 6, or 7; more preferably, m is 1, 3, or 7.

In some embodiments, the chemical linker L has the following structure:

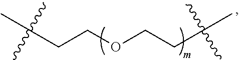

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments of chemical linker L, m is 3.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, the guest molecule A is substituted or unsubstituted adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is substituted or unsubstituted adamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, diamantane, 4,9-diamino diamantane or ferrocene.

In some embodiments, the guest molecule A is substituted or unsubstituted adamantane or diamantane.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, the guest molecule A is substituted with halogen, alkyl, —O-(alkyl), —N-(alkyl).

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

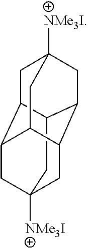

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments, the substituted ferrocene is substituted with C₁-C₆ alkyl, -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

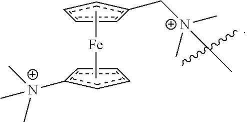

The present invention provides a compound having the structure:

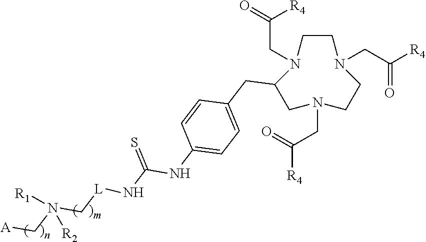

wherein L is a chemical linker;
wherein n and m are each independently 0, 1, 2, 3, 4, 5, or 6;
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

The present invention provides a compound having the structure:

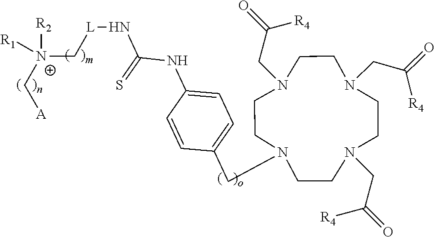

wherein L is a chemical linker;
wherein n and m are each independently 0, 1, 2, 3, 4, 5, or 6;
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH-alkyl, amide.

In some embodiments, each occurrence of R₄ is independently, —OH, —NH₂, alkyl, —O-alkyl, or —NH-alkyl.

In some embodiments, R₄ is —OH.

In some embodiments, R₄ is —NH₂.

In some embodiments, the compound is other than

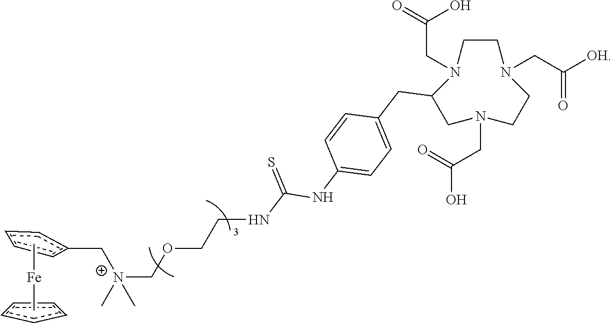

The present invention provides a compound having the structure:

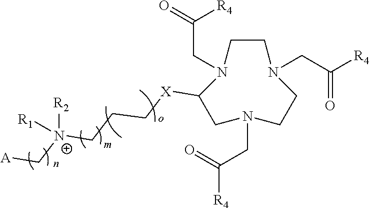

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, or amide.

In some embodiments, each occurrence of R₄ is independently, —OH, —NH₂, alkyl, —O-alkyl, or —NH-alkyl.

In some embodiments, R₄ is —OH.

In some embodiments, R₄ is —NH₂.

The present invention provides a compound having the structure:

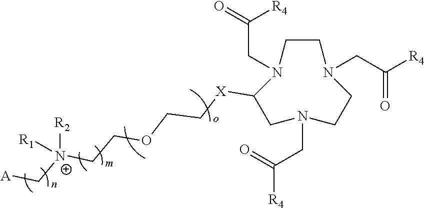

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH-alkyl, amide, aryl, heteroaryl, or alkyl-CF₃.

In some embodiments, each occurrence of R₄ is independently, —OH, —NH₂, alkyl, —O-alkyl, —NH-alkyl, or amide.

In some embodiments, each occurrence of R₄ is independently, —OH, —NH₂, alkyl, —O-alkyl, or —NH-alkyl.

In some embodiments, R₄ is —OH.

In some embodiments, R₄ is —NH₂.

The present invention provides a compound having the structure:

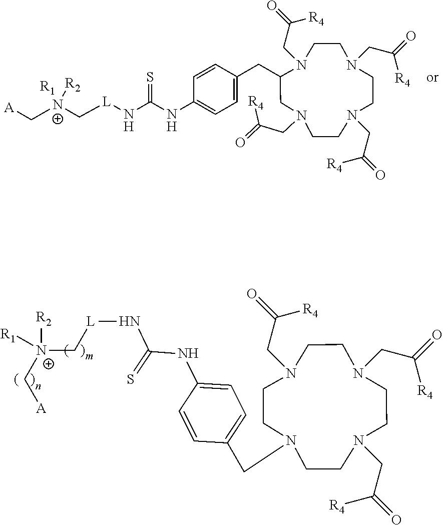

wherein L is a chemical linker;
wherein n and m are each independently 0, 1, 2, 3, 4, 5, or 6;
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

The present invention provides a compound having the structure:

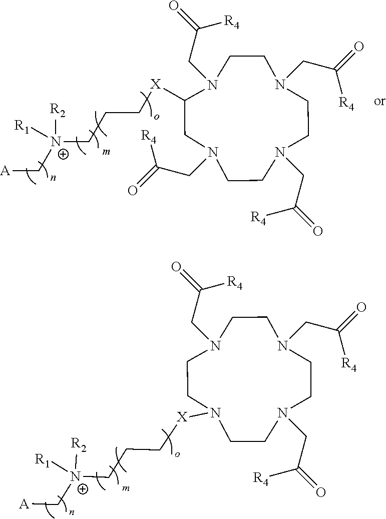

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

The present invention provides a compound having the structure:

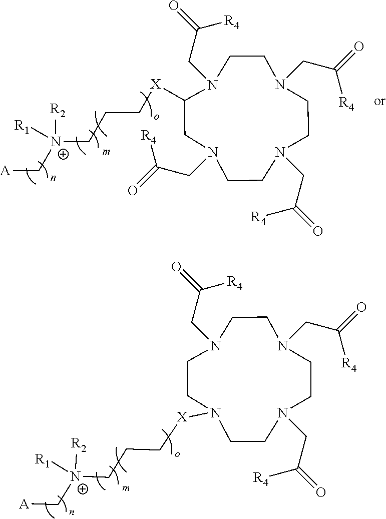

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, or amide.

In some embodiments, each occurrence of R₄ is independently, —OH, —NH₂, alkyl, —O-alkyl, or —NH-alkyl.

In some embodiments, R₄ is —OH.

In some embodiments, R₄ is —NH₂.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

The present invention provides a compound having the structure:

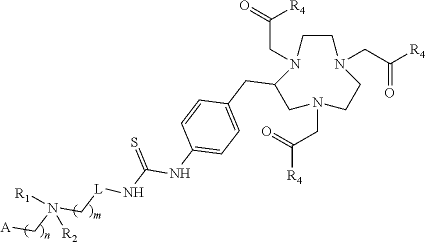

wherein L is alkyl, alkenyl, alkynyl, alkylether, alkylthioether, alkylamino, alkylamido, alkylester, alkylaryl, alklyheteroaryl, or polyethylene glycol (PEG);
wherein n and m are each independently 0, 1, 2, or 3;
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane; and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, the compound is other than

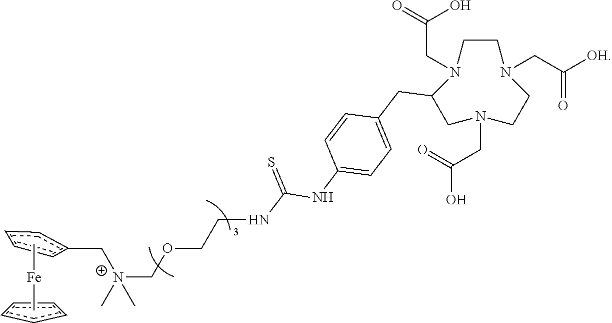

The present invention provides a compound having the structure:

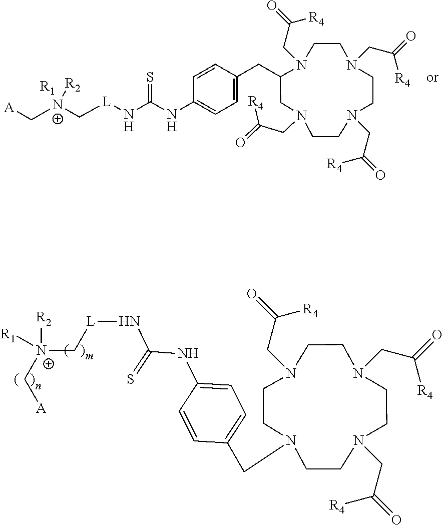

wherein L is alkyl, alkenyl, alkynyl, alkylether, alkylthioether, alkylamino, alkylamido, alkylester, alkylaryl, alklyheteroaryl, or polyethylene glycol (PEG);
wherein n and m are each independently 0, 1, 2, or 3;
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, or amide.

In some embodiments, each occurrence of R₄ is independently, —OH, —NH₂, alkyl, —O-alkyl, or —NH-alkyl.

In some embodiments, R₄ is —OH.

In some embodiments, R₄ is —NH₂.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, alkyl is C₁₋₆ alkyl.

In some embodiments, alkyl is C₁₋₃ alkyl.

In some embodiments, alkyl is methyl.

In some embodiments, aryl is phenyl, p-toluenyl (4-methylphenyl), naphthyl, tetrahydronaphthyl; indanyl, biphenyl, phenanthryl, anthryl or acenaphthyl.

In some embodiments, aryl is phenyl, p-toluenyl (4-methylphenyl), or naphthyl.

In some embodiments, aryl is phenyl.

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

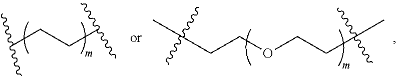

wherein m is 1, 2, 3, 4, 5, 6, 7, 8, 9, or 10; preferably m is 1, 2, 3, 4, 5, 6, or 7; more preferably, m is 1, 3, or 7.

In some embodiments, the chemical linker L has the following structure:

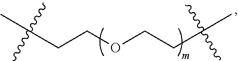

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments of chemical linker L, m is 3.

In some embodiments, the guest molecule A is substituted or unsubstituted adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is substituted or unsubstituted adamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, the guest molecule A is substituted with halogen, alkyl, —O-(alkyl), or —N-(alkyl).

In some embodiments, the guest molecule A is substituted or unsubstituted adamantane, diamantane, 4,9-diamino diamantane or ferrocene.

In some embodiments, the guest molecule A is substituted or unsubstituted adamantane or diamantane.

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

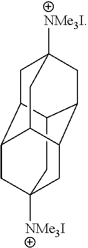

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), or —NH—(C₁-C₆ alkyl).

In some embodiments, the substituted ferrocene is substituted with C₁-C₆ alkyl, or -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

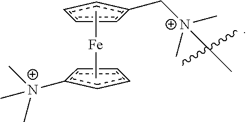

The present invention provides a compound having the structure:

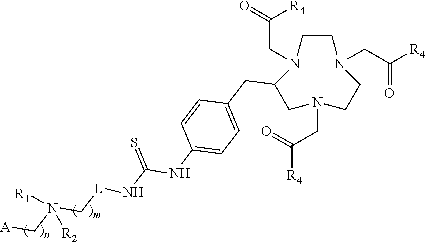

wherein L is alkyl, alkenyl, alkynyl, alkylether, or polyethylene glycol (PEG);
wherein n and m are each independently 1, 2, or 3;
wherein A is a guest molecule which is adamantane, ferrocene, or diamantane; and
wherein R₄ is —OH, —O—(C₁-C₆ alkyl), or NH—(C₁-C₆ alkyl), more preferably R₄ is —OH.

In some embodiments, the compound is other than

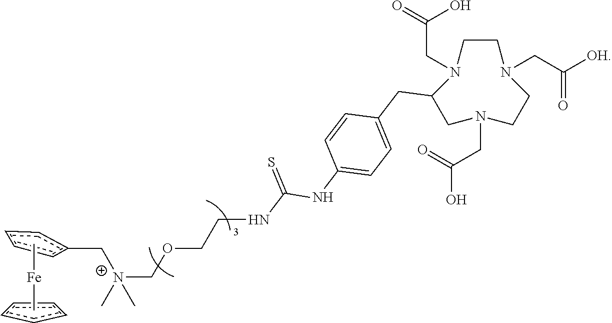

The present invention provides a compound having the structure:

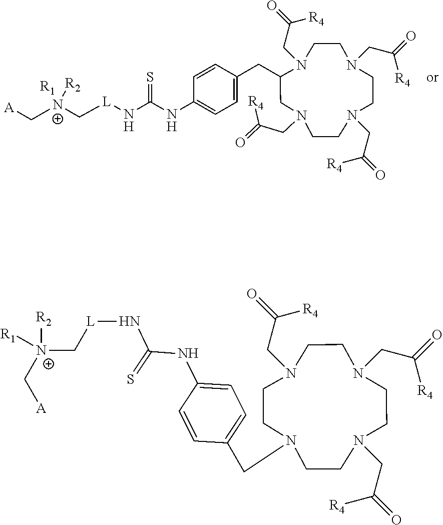

wherein L is alkyl, alkenyl, alkynyl, alkylether, or polyethylene glycol (PEG);
wherein n and m are each independently 1, 2, or 3;
wherein A is a guest molecule which is adamantane, ferrocene, or diamantane; and
wherein R₄ is —OH, —O—(C₁-C₆ alkyl), or NH—(C₁-C₆ alkyl), more preferably R₄ is —OH.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, R₁ and R₂ are ethyl.

The present invention provides compound having the structure:

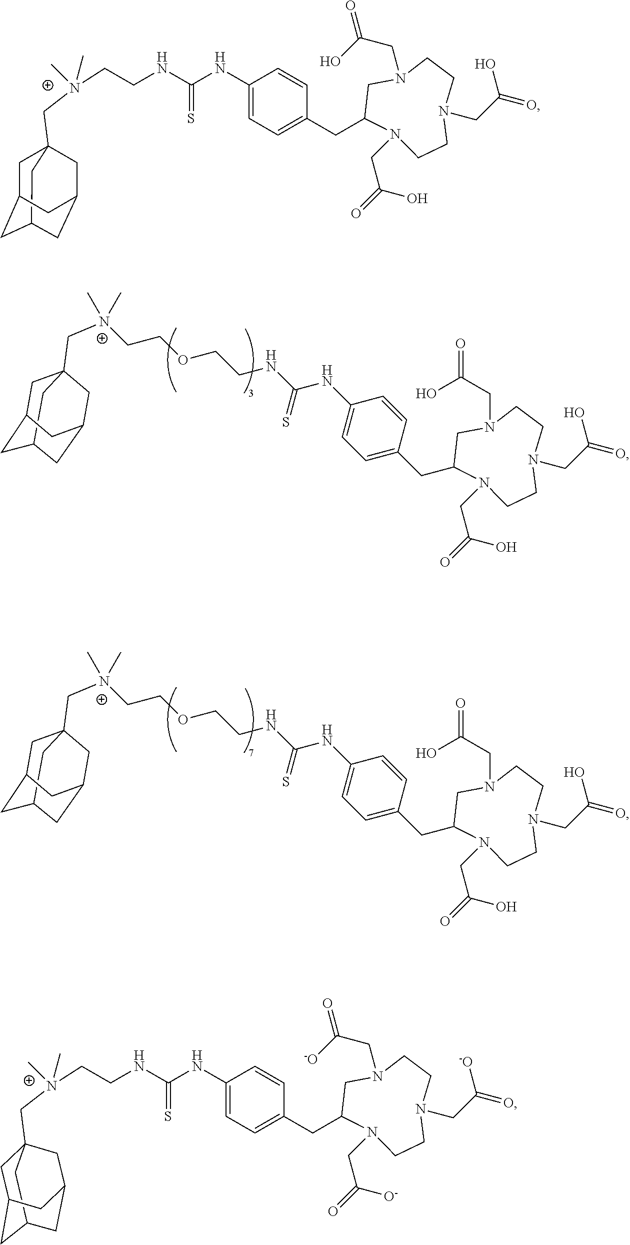

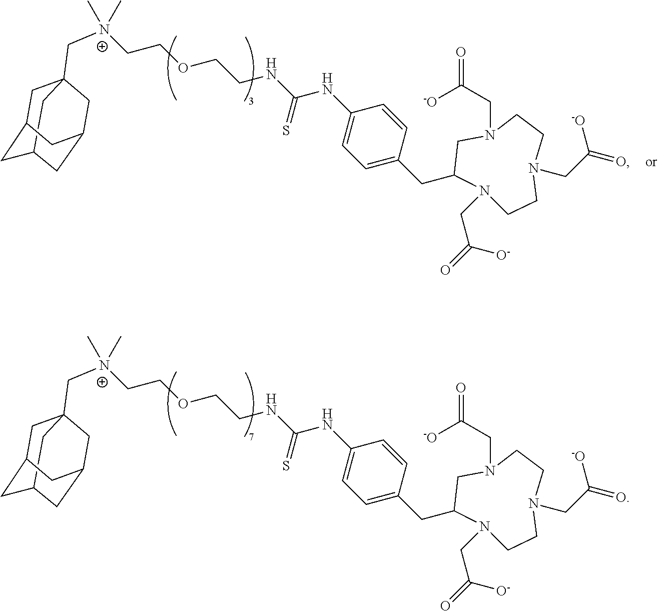

The present invention provides a metal complex comprising the compound described in the invention, wherein the compound coordinates to a metal.

The present invention provides a metal complex having the structure:

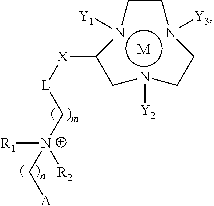

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane, cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; or salt or ester thereof.

The present invention provides a metal complex having the structure:

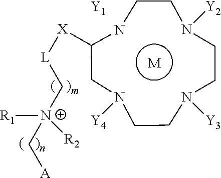

wherein M is the metal;
wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₁ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein n and m are each independently 0, 1, 2, 3, 4, 5, or 6; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane, cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

The present invention provides a metal complex having the structure:

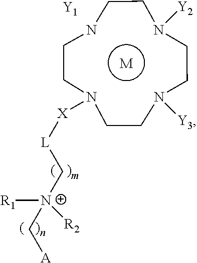

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane, cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, the metal complex is other than

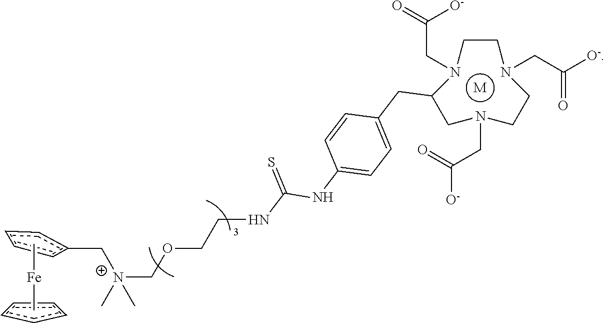

In some embodiments, n and m are each independently 0, 1, 2, 3, 4, 5, or 6.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, R₁ and R₂ are ethyl.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, amide.

In some embodiments, each occurrence of R₄ is independently, —OH, —NH₂, alkyl, —O-alkyl, —NH— alkyl.

In some embodiments, R₄ is —OH.

In some embodiments, R₄ is —NH₂.

In some embodiments, X is alkyl-aryl-thiourea, alkyl-heteroaryl-thiourea, alkyl-cycloalkyl-thiourea, alkenyl-aryl-thiourea, or alkenyl-heteroaryl-thiourea.

In some embodiments, X is alkyl-aryl-thiourea, alkyl-heteroaryl-thiourea, or alkyl-cycloalkyl-thiourea.

In some embodiments, X is alkyl-aryl-thiourea, or alkyl-heteroaryl-thiourea.

In some embodiments, X is alkyl-aryl-thiourea.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃, Y₄ are each independently alkyl-CO₂H, or alkyl-CO₂NH₂, or alkyl-N(alkyl-CO₂H)₂.

In some embodiments, at least one of Y₁, Y₂, Y₃, Y₄ is alkyl-CO₂H.

In some embodiments, at least one of Y₁, Y₂, Y₃, Y₄ is alkyl-CO₂NH₂.

In some embodiments, at least one of Y₁, Y₂, Y₃, Y₄ is alkyl-N(alkyl-CO₂H)₂.

In some embodiments, at least one of Y₁, Y₂, Y₃, Y₄ is —CH₂—CO₂H.

In some embodiments, at least one of Y₁, Y₂, Y₃, Y₄ is —CH₂—CO₂NH₂.

In some embodiments, at least one of Y₁, Y₂, Y₃, Y₄ is —CH₂—N(alkyl-CO₂H)₂

In some embodiments, at least two of Y₁, Y₂ and Y₃, Y₄ are the same.

In some embodiments, at least three of Y₁, Y₂, Y₃ and Y₄ are the same.

In some embodiments, Y₁, Y₂ Y₃, and Y₄ are the same.

In some embodiments, at least one of Y₁, Y₂, Y₃ and Y₄ is H.

In some embodiments, none of Y₁, Y₂, Y₃ and Y₄ are H.

In some embodiments, Y₁, Y₂, Y₃, Y₄ are each independently —H,

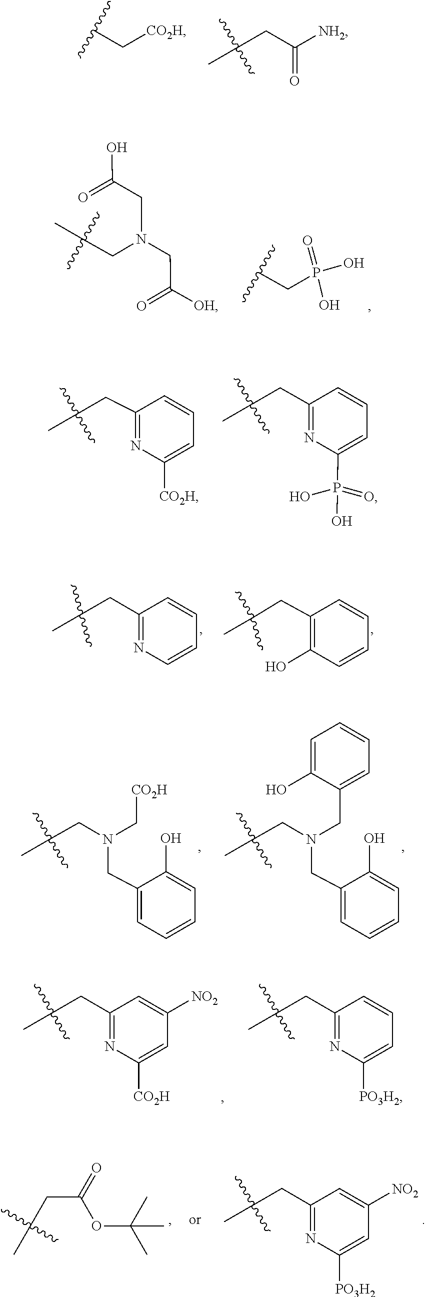

In some embodiments, Y₁, Y₂, Y₃, Y₄ are each independently

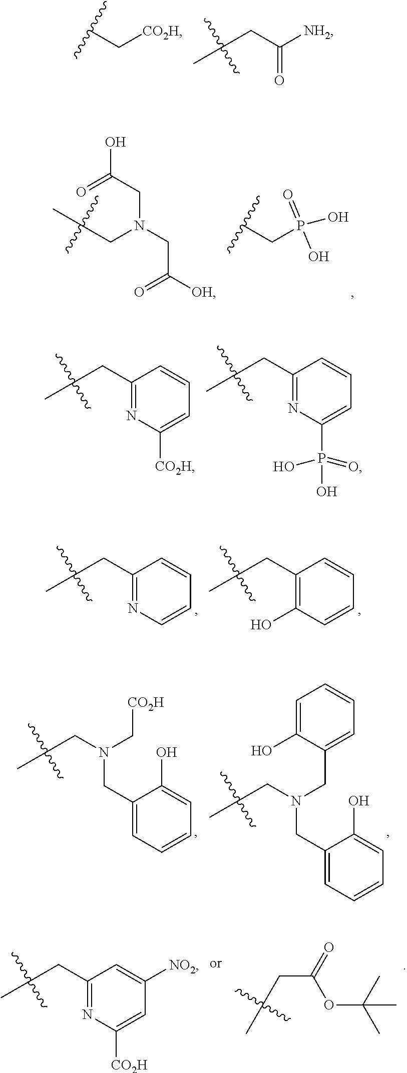

In some embodiments, Y₁, Y₂, Y₃, Y₄ are each independently

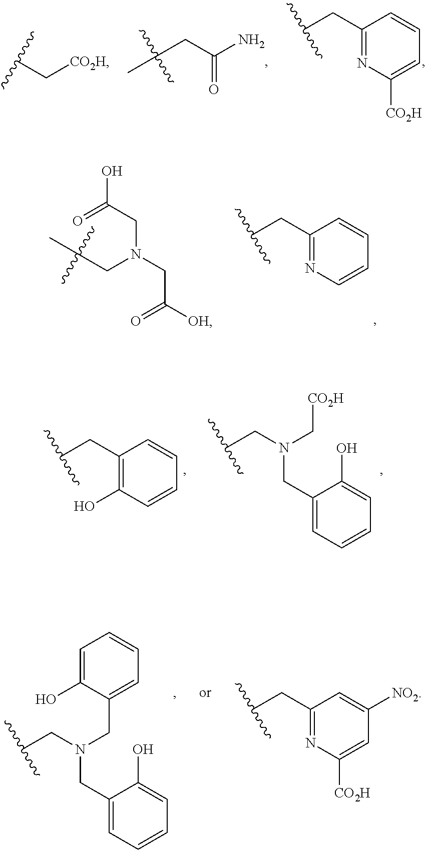

In some embodiments, Y₁, Y₂, Y₃, Y₄ are each independently

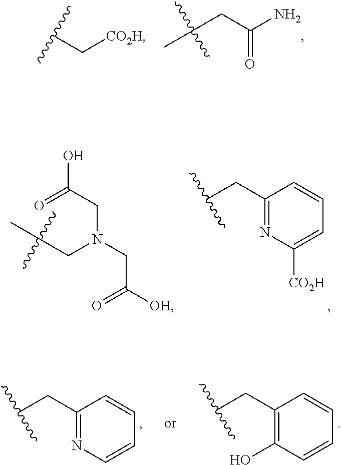 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]

In some embodiments, Y₁, Y₂, Y₃, Y₄ are each independently

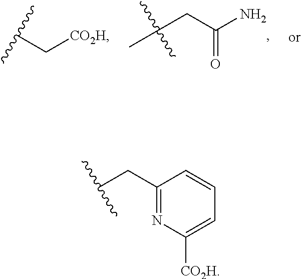 [SMILES: *CC(=O)O ; *CC(N)=O ; *Cc1cccc(C(=O)O)n1]

In some embodiments, Y₁ and Y₃ is

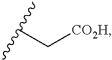 [SMILES: *CC(=O)O]

Y₂ and/or Y₄ is

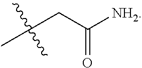 [SMILES: *CC(N)=O]

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

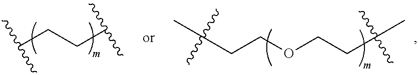

wherein m is 1, 2, 3, 4, 5, 6, 7, 8, 9, or 10; preferably m is 1, 2, 3, 4, 5, 6, or 7; more preferably, m is 1, 3, or 7.

In some embodiments, the chemical linker L has the following structure:

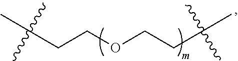

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments, of chemical linker L, m is 3.

In some embodiments, the guest molecule A is adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is adamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, diamantane, 4,9-diamino diamantane or ferrocene.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, the guest molecule A is substituted with halogen, alkyl, —O-(alkyl), —N-(alkyl).

In some embodiments, the guest molecule A is substituted or unsubstituted adamantane or diamantane.

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

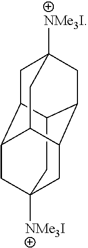

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments, the substituted ferrocene is substituted with C₁-C₆ alkyl, -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

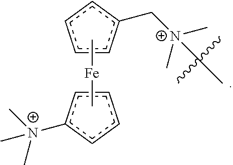

In some embodiments, the present invention provides a metal complex having the structure:

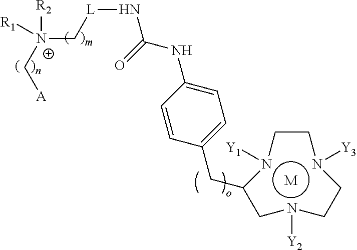

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein o is 0, 1, 2, 3, 4, 5, or 6;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, the present invention provides a metal complex having the structure:

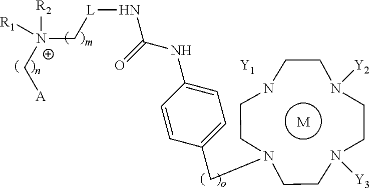

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein o is 0, 1, 2, 3, 4, 5, or 6;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, the metal complex is other than

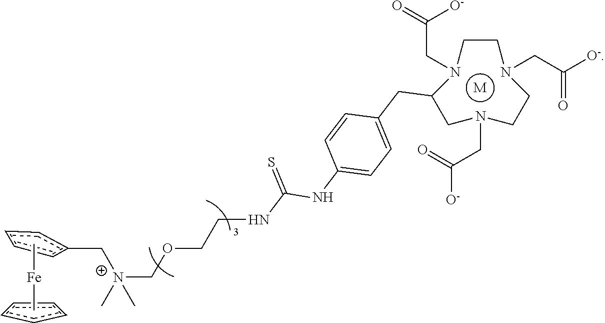

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, amide.

In some embodiments, each occurrence of R₄ is independently, —OH, —NH₂, alkyl, —O-alkyl, —NH— alkyl.

In some embodiments, R₄ is —OH.

In some embodiments, R₄ is —NH₂.

In some embodiments, n and m are each independently 0, 1, 2, 3, 4, 5, or 6.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, o is 0, 1, 2, 3, 4, 5, or 6.

In some embodiments, o is 0, 1, 2, or 3.

In some embodiments, o is 1 or 2.

In some embodiments, o is 1.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃ are each independently alkyl-CO₂H, or alkyl-CO₂NH₂, or alkyl-N(alkyl-CO₂H)₂.

In some embodiments, at least one of Y₁, Y₂, Y₃ is alkyl-CO₂H.

In some embodiments, at least one of Y₁, Y₂, Y₃ is alkyl-CO₂NH₂.

In some embodiments, at least one of Y₁, Y₂, Y₃ is alkyl-N(alkyl-CO₂H)₂.

In some embodiments, at least one of Y₁, Y₂, Y₃ is —CH₂—CO₂H.

In some embodiments, at least one of Y₁, Y₂, Y₃ is —CH₂—CO₂NH₂.

In some embodiments, at least one of Y₁, Y₂, Y₃ is —CH₂—N(alkyl-CO₂H)₂

In some embodiments, at least two of Y₁, Y₂ and Y₃ are the same.

In some embodiments, at least three of Y₁, Y₂, Y₃ and Y₄ are the same.

In some embodiments, Y₁, Y₂ and Y₃ are the same.

In some embodiments, at least one of Y₁, Y₂, and Y₃ is H.

In some embodiments, none of Y₁, Y₂, and Y₃ are H.

In some embodiments, Y₁, Y₂, Y₃, Y₄ are each independently —H,

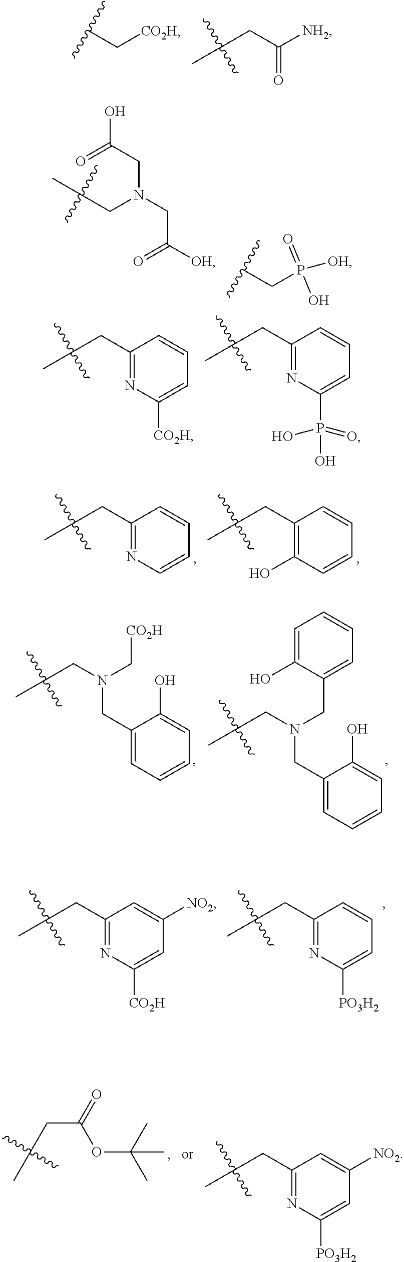

In some embodiments, Y₁, Y₂, Y₃, Y₄ are each independently

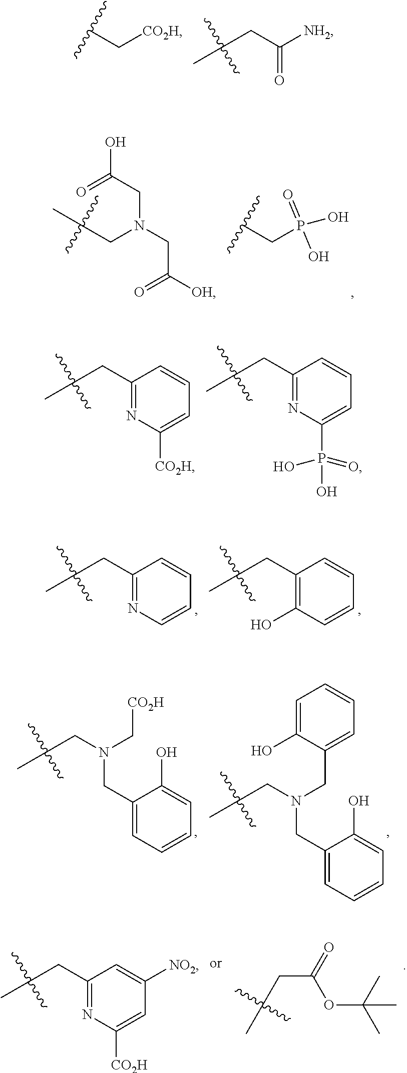

In some embodiments, Y₁, Y₂, Y₃, Y₄ are each independently

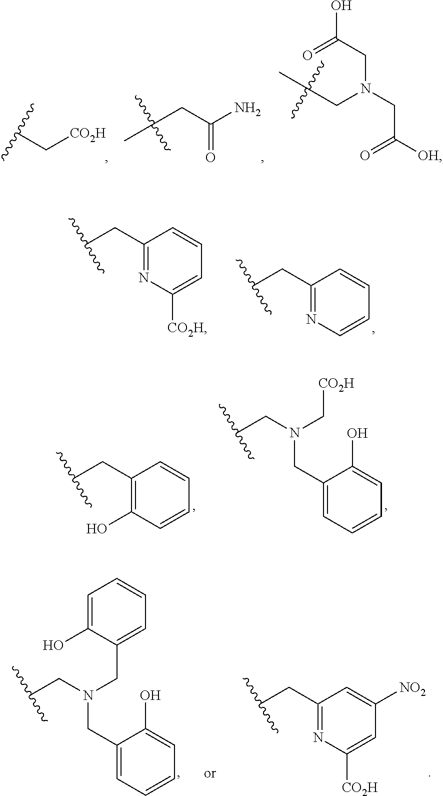

In some embodiments, Y₁, Y₂, Y₃, Y₄ are each independently

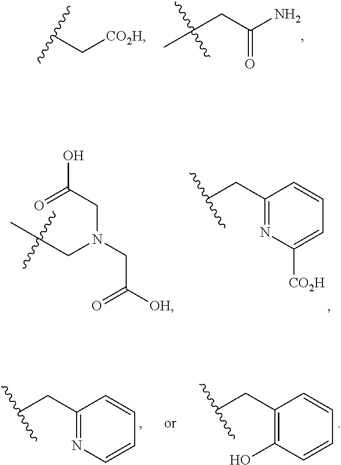 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]

In some embodiments, Y₁, Y₂, Y₃, Y₄ are each independently

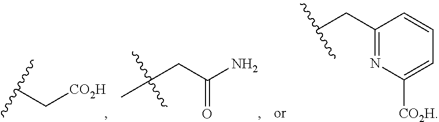 [SMILES: *CC(=O)O ; *CC(N)=O ; *Cc1cccc(C(=O)O)n1]

In some embodiments, Y₁ and Y₃ is

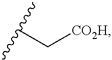 [SMILES: *CC(=O)O]

Y₂ and/or Y₄ is

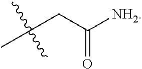 [SMILES: *CC(N)=O]

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

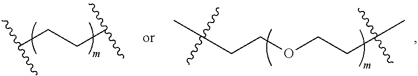

wherein m is 1, 2, 3, 4, 5, 6, 7, 8, 9, or 10; preferably m is 1, 2, 3, 4, 5, 6, or 7; more preferably, m is 1, 3, or 7.

In some embodiments, the chemical linker L has the following structure:

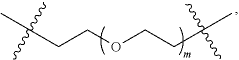

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments, of chemical linker L, m is 3.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, the guest molecule A is adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is adamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, diamantane, 4,9-diamino diamantane or ferrocene.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, the guest molecule A is substituted with halogen, alkyl, —O-(alkyl), —N-(alkyl).

In some embodiments, the guest molecule A is substituted or unsubstituted adamantane or diamantane.

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

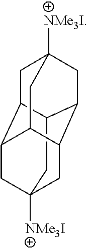

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments, the substituted ferrocene is substituted with C₁-C₆ alkyl, -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

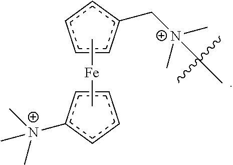

In some embodiments, the present invention provides a metal complex having the structure:

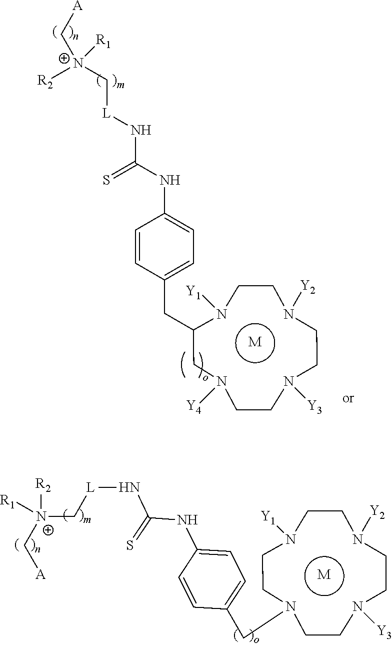

wherein M is the metal;
wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, amide.

In some embodiments, each occurrence of R₄ is independently, —OH, —NH₂, alkyl, —O-alkyl, —NH— alkyl.

In some embodiments, R₄ is —OH.

In some embodiments, R₄ is —NH₂.

In some embodiments, n and m are each independently 0, 1, 2, 3, 4, 5, or 6.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, o is 0, 1, 2, 3, 4, 5, or 6.

In some embodiments, o is 0, 1, 2, or 3.

In some embodiments, o is 1 or 2.

In some embodiments, o is 1.

In some embodiments, R₁ and R₂ are each independently H, halogen, alkyl, alkenyl, alkynyl, —OH, —O-(alkyl), —CHF₂, —CF₃, —OCHF₂ or —OCF₃.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, Y₁, Y₂, Y₃, Y₄ are each independently alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃, Y₄ are each independently alkyl-CO₂H, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments, Y₁, Y₂, Y₃, Y₄ are each independently alkyl-CO₂H, or alkyl-CO₂NH₂, or alkyl-N(alkyl-CO₂H)₂.

In some embodiments, at least one of Y₁, Y₂, Y₃, Y₄ is alkyl-CO₂H.

In some embodiments, at least one of Y₁, Y₂, Y₃, Y₄ is alkyl-CO₂NH₂.

In some embodiments, at least one of Y₁, Y₂, Y₃, Y₄ is alkyl-N(alkyl-CO₂H)₂.

In some embodiments, at least one of Y₁, Y₂, Y₃, Y₄ is —CH₂—CO₂H.

In some embodiments, at least one of Y₁, Y₂, Y₃, Y₄ is —CH₂—CO₂NH₂.

In some embodiments, at least one of Y₁, Y₂, Y₃, Y₄ is —CH₂—N(alkyl-CO₂H)₂

In some embodiments, at least two of Y₁, Y₂ and Y₃, Y₄ are the same.

In some embodiments, at least three of Y₁, Y₂, Y₃ and Y₄ are the same.

In some embodiments, Y₁, Y₂ Y₃, and Y₄ are the same.

In some embodiments, Y₁, Y₂, Y₃, Y₄ are each independently —H,

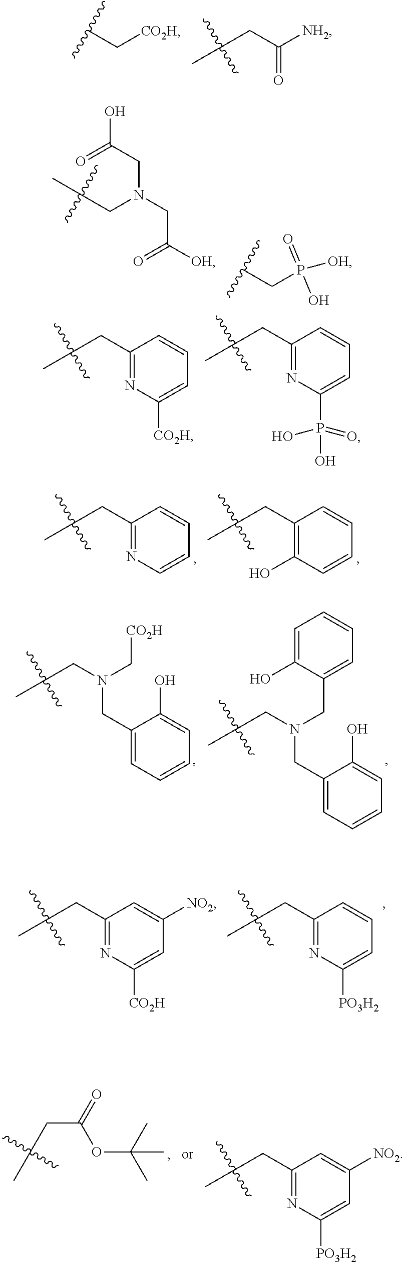

In some embodiments, Y₁, Y₂, Y₃, Y₄ are each independently

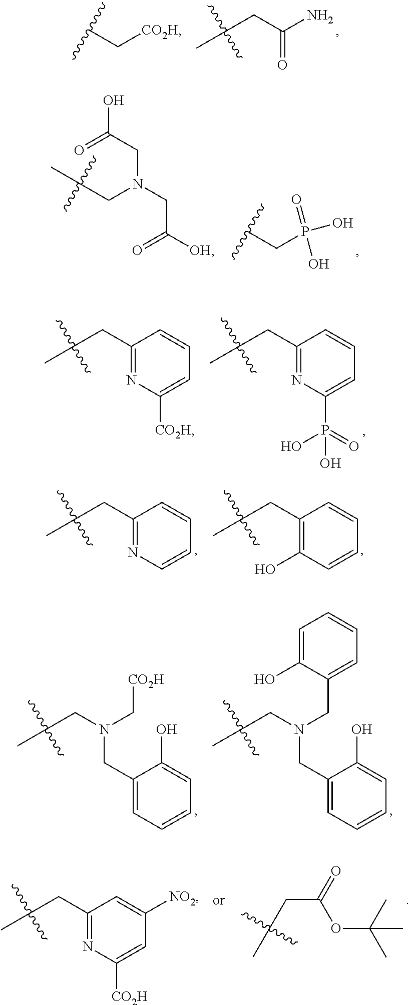

In some embodiments, Y₁, Y₂, Y₃, Y₄ are each independently

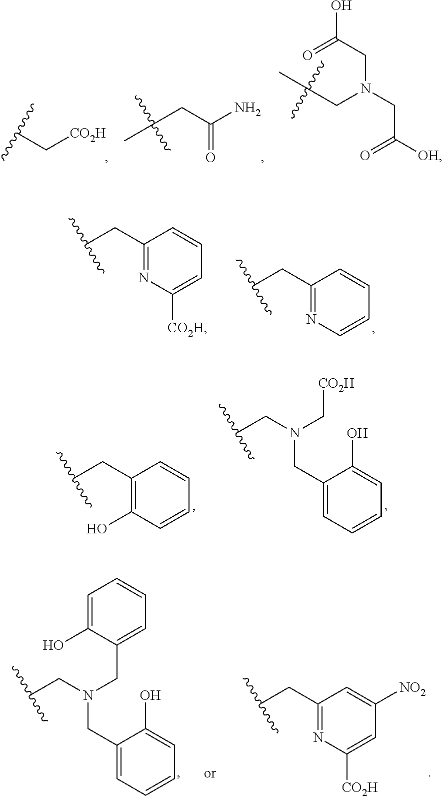

In some embodiments, Y₁, Y₂, Y₃, Y₄ are each independently

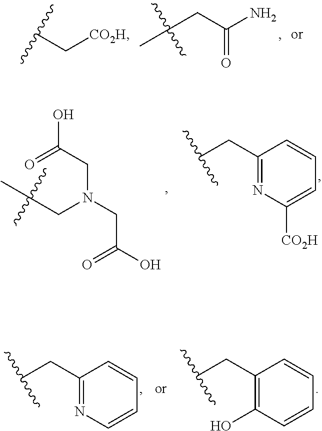 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]

In some embodiments, Y₁, Y₂, Y₃, Y₄ are each independently

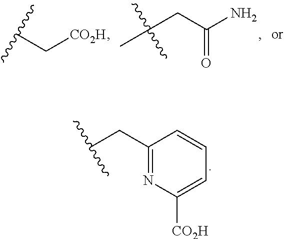 [SMILES: *CC(=O)O ; *CC(N)=O ; *Cc1cccc(C(=O)O)n1]

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

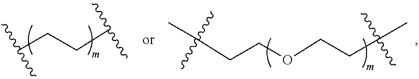

wherein m is 1, 2, 3, 4, 5, 6, 7, 8, 9, or 10; preferably m is 1, 2, 3, 4, 5, 6, or 7; more preferably, m is 1, 3, or 7.

In some embodiments, the chemical linker L has the following structure:

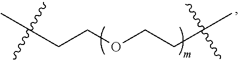

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments, of chemical linker L, m is 3.

In some embodiments, the guest molecule A is adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is adamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, diamantane, 4,9-diamino diamantane or ferrocene.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, the guest molecule A is substituted with halogen, alkyl, —O-(alkyl), —N-(alkyl).

In some embodiments, the guest molecule A is substituted or unsubstituted adamantane or diamantane.

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

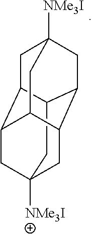

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments, the substituted ferrocene is substituted with C₁-C₆ alkyl, -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

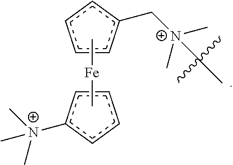

The present invention provides a metal complex having the structure:

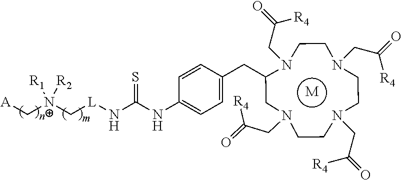

wherein M is the metal;
wherein L is a chemical linker;
wherein n and m are each independently 0, 1, 2, 3, 4, 5, or 6; preferably, n and m are each independently 1, 2, or 3; more preferably, n and m are 1;
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

The present invention provides a metal complex having the structure:

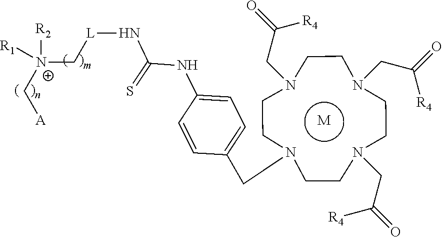

wherein M is the metal;
wherein L is a chemical linker;
wherein n and m are each independently 0, 1, 2, 3, 4, 5, or 6; preferably, n and m are each independently 1, 2, or 3; more preferably, n and m are 1;
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

The present invention provides a metal complex having the structure:

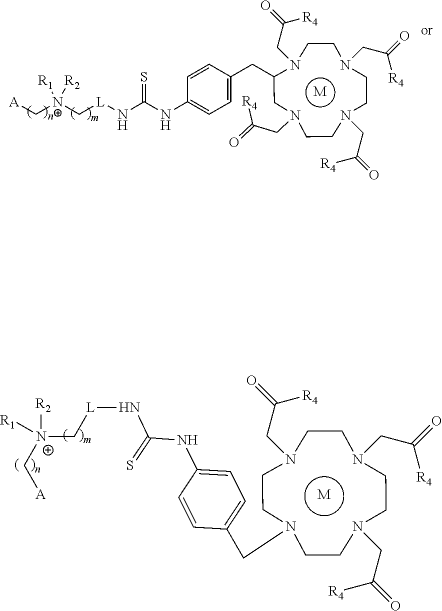

wherein M is the metal;
wherein L is an alkyl linker, an alkyne linker, alkynal linker or a polyethylene glycol (PEG) or combinations thereof;
wherein n and m are each independently 0, 1, 2, 3, 4, 5, or 6; preferably, n and m are each independently 1, 2, or 3; more preferably, n and m are 1;
wherein A is a guest molecule which is adamantane, ferrocene, diamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, or amide.

In some embodiments, each occurrence of R₄ is independently, —OH, —NH₂, alkyl, —O-alkyl, or —NH-alkyl.

In some embodiments, R₄ is —OH.

In some embodiments, R₄ is —NH₂.

In some embodiments, n and m are each independently 0, 1, 2, 3, 4, 5, or 6.

In some embodiments, n and m are each independently 1, 2, or 3.

In some embodiments, n and m are 1.

In some embodiments, n is 1, 2, or 3.

In some embodiments, m is 1, 2, or 3.

In some embodiments, n is 1 or 2.

In some embodiments, m is 1 or 2.

In some embodiments, n is 1.

In some embodiments, m is 1.

In some embodiments, n and m are the same.

In some embodiments, n and m are different.

In some embodiments, R₁ and R₂ are each independently H, halogen, C₁-C₆ alkyl, C₁-C₆ alkenyl, or C₁-C₆ alkynyl.

In some embodiments, R₁ and R₂ are each independently, C₁-C₆ alkyl or C₁-C₆ alkenyl.

In some embodiments, R₁ and R₂ are each independently C₁-C₆ alkyl.

In some embodiments, R₁ and R₂ are C₁₋₅ alkyl.

In some embodiments, R₁ and R₂ are C₁₋₃ alkyl.

In some embodiments, R₁ and R₂ are methyl.

In some embodiments, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments, the chemical linker L is an alkyl linker, an alkyne linker, alkynal linker or a polyethylene glycol (PEG) or combinations thereof.

In some embodiments, the chemical linker L is an alkyl or a PEG or combinations thereof.

In some embodiments, the chemical linker L is a PEG.

In some embodiments, the chemical linker L has the following structure:

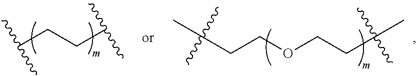

wherein m is 1, 2, 3, 4, 5, 6, 7, 8, 9, or 10; preferably m is 1, 2, 3, 4, 5, 6, or 7; more preferably, m is 1, 3, or 7.

In some embodiments, the chemical linker L has the following structure:

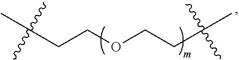

wherein m is 1, 2, 3, 4, 5, 6, 7, 8, 9, or 10; preferably m is 1, 2, 3, 4, 5, 6, or 7; more preferably, m is 1, 3, or 7.

In some embodiments of chemical linker L, m is 1, 3 or 7.

In some embodiments, of chemical linker L, m is 3.

In some embodiments, alkyl is C₁₋₆ alkyl.

In some embodiments, alkyl is C₁₋₃ alkyl.

In some embodiments, alkyl is methyl.

In some embodiments, aryl is phenyl, p-toluenyl (4-methylphenyl), naphthyl, tetrahydronaphthyl; indanyl, biphenyl, phenanthryl, anthryl or acenaphthyl.

In some embodiments, aryl is phenyl, p-toluenyl (4-methylphenyl), or naphthyl.

In some embodiments, aryl is phenyl.

In some embodiments, the guest molecule A is adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments, the guest molecule A is adamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments, the guest molecule A is substituted or unsubstituted adamantane, diamantane, 4,9-diamino diamantane or ferrocene.

In some embodiments, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments, the guest molecule A is substituted with halogen, alkyl, —O-(alkyl), —N-(alkyl).

In some embodiments, the guest molecule A is substituted or unsubstituted adamantane or diamantane.

In some embodiments, the guest molecule A is substituted or unsubstituted adamantane.

In some embodiments, the guest molecule A is substituted or unsubstituted diamantane.

In some embodiments, the guest molecule A is substituted or unsubstituted ferrocene.

In some embodiments, the guest molecule A is unsubstituted adamantane.

In some embodiments, the guest molecule A is substituted adamantane.

In some embodiments, the guest molecule A is 4,9-diamino diamantane.

In some embodiments, the guest molecule A is unsubstituted ferrocene.

In some embodiments, the guest molecule A is unsubstituted diamantane.

In some embodiments, the guest molecule A is substituted diamantane.

In some embodiments, the substituted diamantane having the following structure:

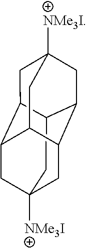

In some embodiments, the guest molecule A is substituted ferrocene.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments, the substituted ferrocene is substituted with C₁-C₆ alkyl, -alkyl-N—(C₁-C₆ alkyl).

In some embodiments, the substituted ferrocene is substituted with -alkyl-N—(C₁-C₆ alkyl).

In some embodiments, the substituted ferrocene having the following structure:

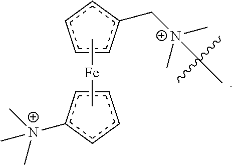

The present invention provides a metal complex having the structure:

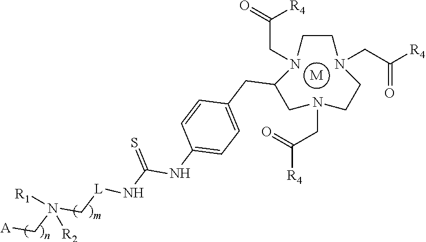

wherein M is the metal;
wherein L is a chemical linker;
wherein n and m are each independently 1, 2, or 3;
wherein A is a guest molecule which is adamantane, ferrocene, or diamantane; and
wherein R₄ is —OH, —NH₂, —O—(C₁-C₆ alkyl), or NH—(C₁-C₆ alkyl), more preferably R₄ is —OH or —NH₂.

The present invention provides a metal complex having the structure:

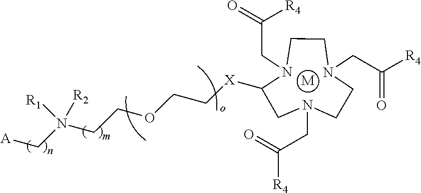

wherein \textcircled{M} is the metal;
wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

The present invention provides a metal complex having the structure:

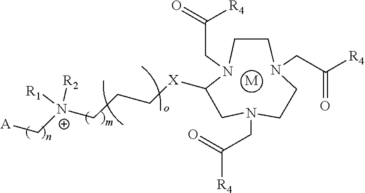

wherein \textcircled{M} is the metal;
wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

The present invention provides a metal complex having the structure:

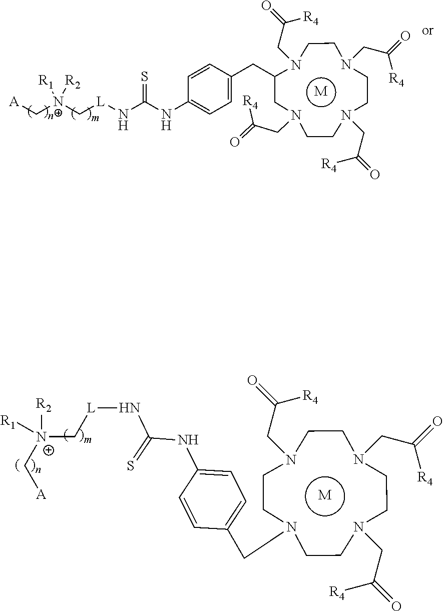

wherein M is the metal;
wherein L is a chemical linker;
wherein n and m are each independently 1, 2, or 3;
wherein A is a guest molecule which is adamantane, ferrocene, or diamantane; and
wherein each occurrence of R₄ is independently —OH, —NH₂, —O—(C₁-C₆ alkyl), or NH—(C₁-C₆ alkyl), more preferably R₄ is —OH or —NH₂.

The present invention provides a metal complex having the structure:

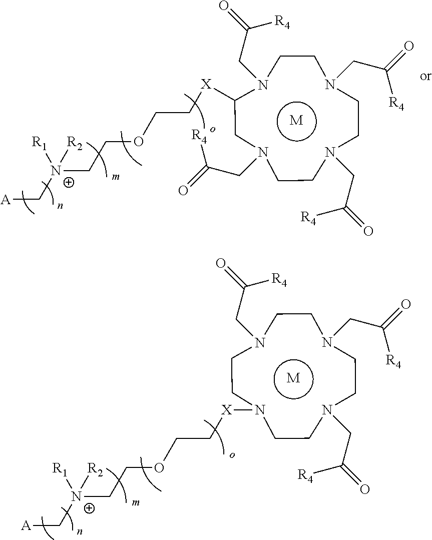

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
wherein R₄ is independently, —H, alkyl, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; or salt or ester thereof.

The present invention provides metal complex having the structure:

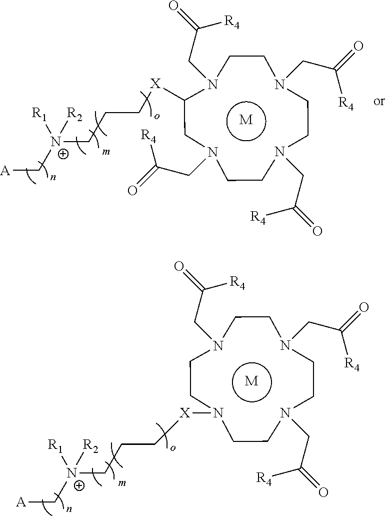

wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1;
wherein each occurrence of R₄ is independently —OH, —NH₂, —O—(C₁-C₆ alkyl), or NH—(C₁-C₆ alkyl), more preferably R₄ is —OH or —NH₂;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein R₁ and R₂ are each independently H, halogen, alkyl, alkenyl, alkynyl, —OH, —O-(alkyl), —CHF₂, —CF₃, —OCHF₂ or —OCF₃; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, A is substituted with —NH₂, -alkyl-NH₂, —NH-alkyl, or alkyl.

In some embodiments, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments, each occurrence of R₄ is independently, —OH, —NH₂, alkyl, —O-alkyl, —NH— alkyl, amide, aryl, heteroaryl, or alkyl-CF₃.

In some embodiments, each occurrence of R₄ is independently, —OH, —NH₂, alkyl, —O-alkyl, —NH— alkyl, or amide.

In some embodiments, each occurrence of R₄ is independently, —OH, —NH₂, alkyl, —O-alkyl, or —NH-alkyl.

The present invention provides a metal complex having the structure:

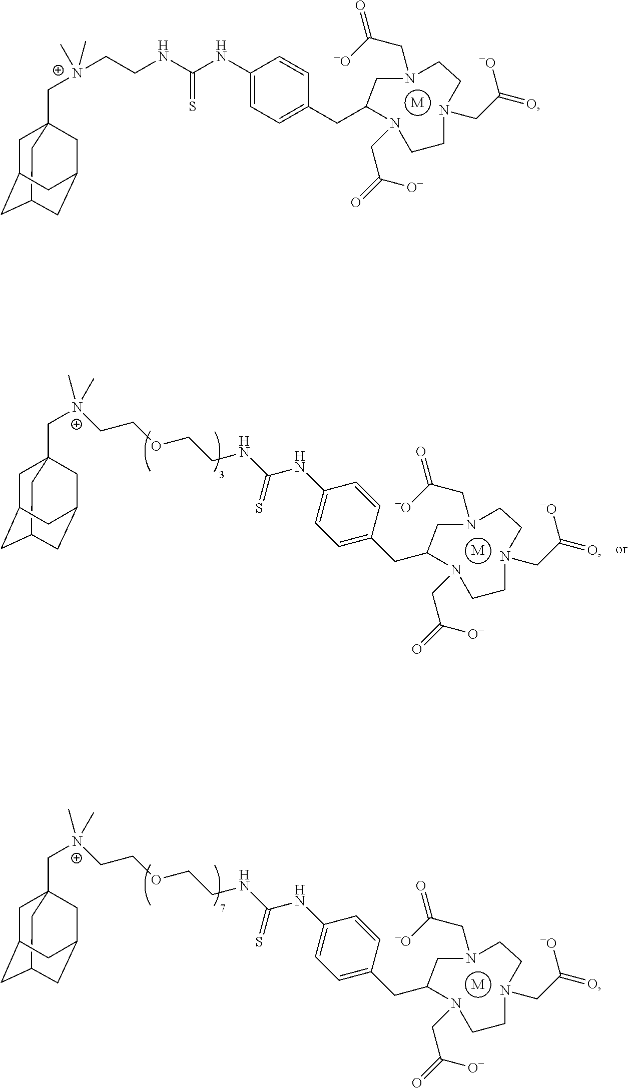

wherein \textcircled{M} is the metal.

In some embodiments, the metal is Copper-62 (⁶²Cu), Copper-64 (⁶⁴Cu), Copper-67 (⁶⁷Cu), Gallium-68 (⁶⁸Ga) Scandium-44 (⁴⁴Sc), Scandium-47 (⁴⁷Sc), Scandium-43 (⁴³Sc), Lead-203 (²⁰³Pb), Lead-212 (²¹²Pb), Lanthanum-132 (¹³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb)

In some embodiments, the metal is Copper-62 (⁶²Cu), Copper-64 (⁶⁴Cu), Copper-67 (⁶⁷Cu), Scandium-44 (⁴⁴Sc), Scandium-47 (⁴⁷Sc), or Scandium-43 (⁴³Sc).

In some embodiments, the metal is Copper-64 (⁶⁴Cu).

The present invention provides a pharmaceutical composition comprising the metal complex described in the invention and a marker attached to a host molecule.

In some embodiments, the marker is a biological marker.

In some embodiments, the marker is modified.

In some embodiments, the marker is un-modified.

In some embodiments, the marker is a tumor marker or a cancer marker.

In some embodiments, the tumor marker is a prostate-specific antigen (PSA), prostatic acid phosphatase (PAP), cancer antigen 125 (CA 125), carcinoembryonic antigen (CEA), alpha-fetoprotein (AFP), human chorionic gonadotropin (HCG), cancer antigen 19-9 (CA 19-9), cancer antigen 15-3 (CA 15-3), cancer antigen 27-29 (CA 27-29), lactate dehydrogenase (LDH), or neuron-specific enolase (NSE).

In some embodiments, the tumor marker is prostate-specific antigen (PSA), cancer antigen 125 (CA 125), carcinoembryonic antigen (CEA), cancer antigen 19-9 (CA 19-9), cancer antigen 15-3 (CA 15-3), or cancer antigen 27-29 (CA 27-29).

In some embodiments, the tumor marker is a prostate-specific antigen (PSA) or carcinoembryonic antigen (CEA).

In some embodiments, the tumor marker is a carcinoembryonic antigen (CEA).

In some embodiments, the host molecule comprises cucurbit[5]uril, cucurbit[6]uril, cucurbit[7]uril, cucurbit[8]uril, cucurbit[10]uril, cucurbit[14]uril, cyclodextrin, or calix-[5]-arenes.

In some embodiments, the host molecule comprises cucurbit[5]uril, cucurbit[6]uril, cucurbit[7]uril, cucurbit[8]uril, or cucurbit[10]uril.

In some embodiments, the host molecule comprises cucurbit[5]uril, cucurbit[6]uril, cucurbit[7]uril, or cucurbit[8]uril.

In some embodiments, the host molecule comprises cucurbit[7]uril, or cucurbit[8]uril.

In some embodiments, the host molecule is cucurbit[7]uril.

In some embodiments, the interaction between the host and the guest molecule is a non-covalent interaction.

In some embodiments, the non-covalent interaction is ion-ion interaction, ion-dipole interaction, dipole-dipole interaction, hydrogen bonding, cation-7 interaction, 1-1 interaction, van der Waals interaction or hydrophobic interaction.

In some embodiments, the non-covalent interaction is ion-ion interaction, or an der Waals interaction.

In some embodiments, the metal complex described in the invention and the host molecule described in the invention form a high affinity host-guest complex.

The present invention provides a method of detecting cells in a subject comprises administering an effective amount of metal complex having the structure:

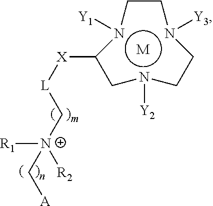

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof,
to the subject who contains a marker attached to a host molecule.

The present invention provides a method of detecting cells in a subject comprises administering an effective amount of metal complex having the structure:

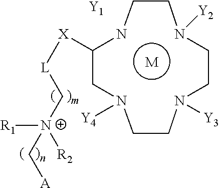

wherein M is the metal;
wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein n and m are each independently 0, 1, 2, 3, 4, 5, or 6; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof,
to the subject who contains a marker attached to a host molecule.

The present invention provides a method of detecting cells in a subject comprises administering an effective amount of metal complex having the structure:

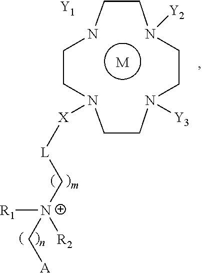

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof,
to the subject who contains a marker attached to a host molecule.

In some embodiments of the method, the metal complex is other than

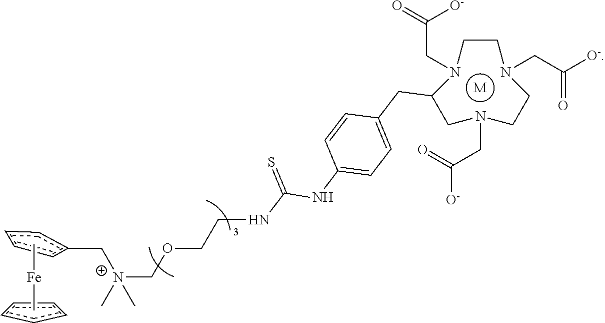

In some embodiments of the method, A is substituted with —OH, —NH₂, halogen, alkyl, —O-alkyl, -alkyl-NH₂, —NH-alkyl, —CHF₂, —CF₃, —OCHF₂, —OCF₃.

In some embodiments of the method, A is substituted with —NH₂, -alkyl-NH₂, —NH-alkyl, or alkyl.

In some embodiments of the method, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —OH, —NH₂, alkyl, —O-alkyl, —NH-alkyl, amide, aryl, heteroaryl, or alkyl-CF₃.

In some embodiments of the method, each occurrence of R₄ is independently, —OH, —NH₂, alkyl, —O-alkyl, —NH-alkyl, amide.

In some embodiments of the method, each occurrence of R₄ is independently, —OH, —NH₂, alkyl, —O-alkyl, —NH-alkyl.

In some embodiments of the method, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments of the method, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments of the method, Y₁, Y₂, Y₃ are each independently alkyl-CO₂H, or alkyl-CO₂NH₂, or alkyl-N(alkyl-CO₂H)₂.

In some embodiments of the method, at least one of Y₁, Y₂, Y₃ is alkyl-CO₂H.

In some embodiments of the method, at least one of Y₁, Y₂, Y₃ is alkyl-CO₂NH₂.

In some embodiments of the method, at least one of Y₁, Y₂, Y₃ is alkyl-N(alkyl-CO₂H)₂.

In some embodiments of the method, at least one of Y₁, Y₂, Y₃ is —CH₂—CO₂H.

In some embodiments of the method, at least one of Y₁, Y₂, Y₃ is —CH₂—CO₂NH₂.

In some embodiments of the method, at least one of Y₁, Y₂, Y₃ is —CH₂—N(alkyl-CO₂H)₂

In some embodiments of the method, at least two of Y₁, Y₂ and Y₃ are the same.

In some embodiments of the method, Y₁, Y₂, and Y₃ are the same.

In some embodiments of the method, at least one of Y₁, Y₂, Y₃ and Y₄ is H.

In some embodiments of the method, none of Y₁, Y₂, Y₃ and Y₄ are H.

In some embodiments of the method, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments of the method, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments of the method, Y₁, Y₂, Y₃, Y₄ are each independently alkyl-CO₂H, or alkyl-CO₂NH₂, or alkyl-N(alkyl-CO₂H)₂.

In some embodiments of the method, at least one of Y₁, Y₂, Y₃, Y₄ is alkyl-CO₂H.

In some embodiments of the method, at least one of Y₁, Y₂, Y₃, Y₄ is alkyl-CO₂NH₂.

In some embodiments of the method, at least one of Y₁, Y₂, Y₃, Y₄ is alkyl-N(alkyl-CO₂H)₂.

In some embodiments of the method, at least one of Y₁, Y₂, Y₃, Y₄ is —CH₂—CO₂H.

In some embodiments of the method, at least one of Y₁, Y₂, Y₃, Y₄ is —CH₂—CO₂NH₂.

In some embodiments of the method, at least one of Y₁, Y₂, Y₃, Y₄ is —CH₂—N(alkyl-CO₂H)₂

In some embodiments of the method, at least two of Y₁, Y₂, Y₃, Y₄ are the same.

In some embodiments of the method, at least three of Y₁, Y₂, Y₃, Y₄ are the same.

In some embodiments of the method, Y₁, Y₂, Y₃, Y₄ are the same.

In some embodiments of the method, at least one of Y₁, Y₂, Y₃ and Y₄ is H.

In some embodiments of the method, none of Y₁, Y₂, Y₃ and Y₄ are H.

In some embodiments of the method, Y₁, Y₂, Y₃, Y₄ are each independently —H,

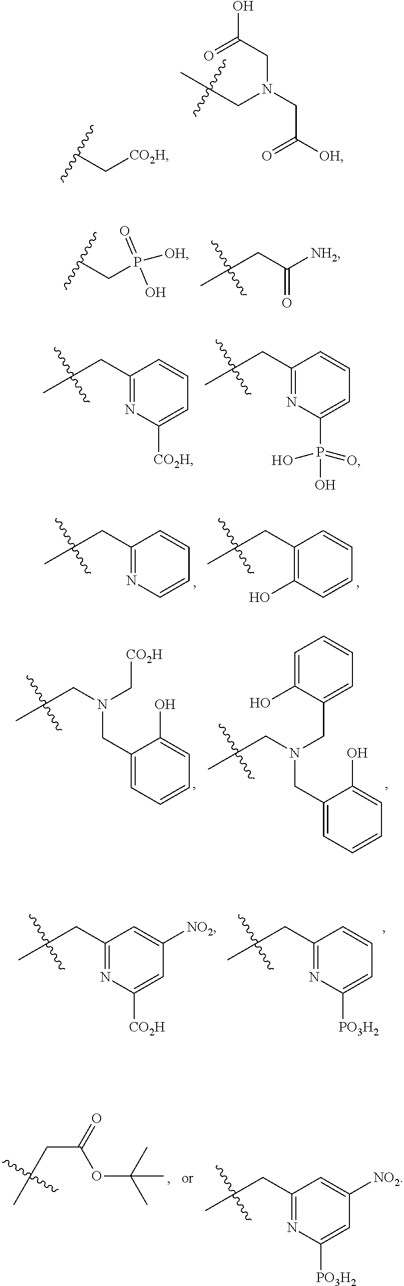

In some embodiments of the method, Y₁, Y₂, Y₃, Y₄ are each independently

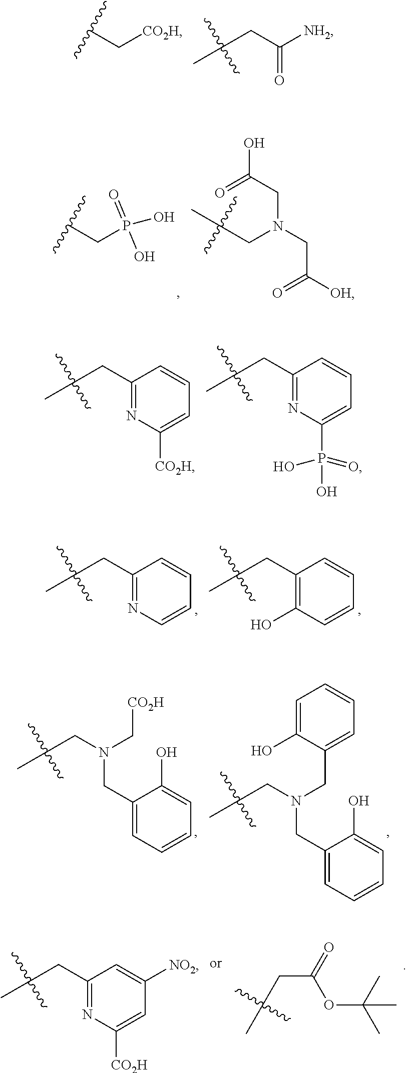

In some embodiments of the method, Y₁, Y₂, Y₃, Y₄ are each independently

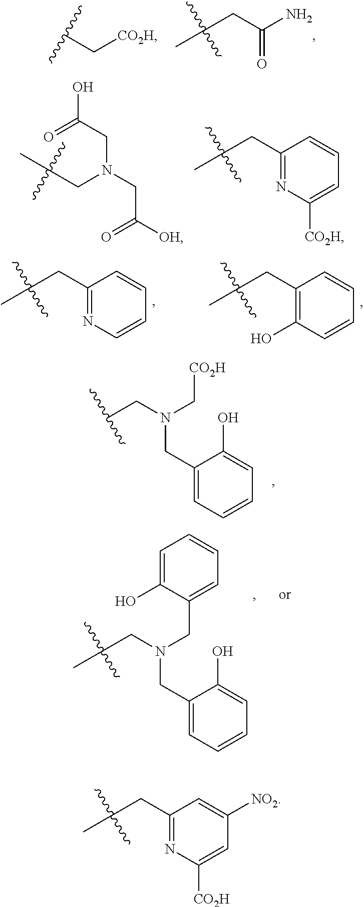

In some embodiments of the method, Y₁, Y₂, Y₃, Y₄ are each independently

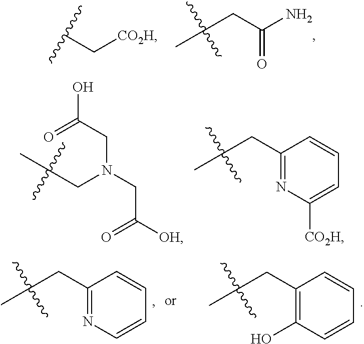

In some embodiments of the method, Y₁, Y₂, Y₃, Y₄ are each independently

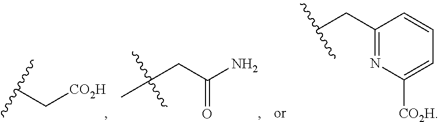 [SMILES: *CC(=O)O ; *CC(N)=O ; *Cc1cccc(C(=O)O)n1]

In some embodiments, Y₁ and Y₃ is

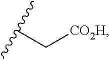 [SMILES: *CC(=O)O]

Y₂ and/or Y₄ is

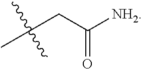 [SMILES: *CC(N)=O]

In some embodiments of the method, n and m are each independently 0, 1, 2, 3, 4, 5, or 6.

In some embodiments of the method, n and m are each independently 1, 2, or 3.

In some embodiments of the method, n and m are 1.

In some embodiments of the method, n is 1, 2, or 3.

In some embodiments of the method, m is 1, 2, or 3.

In some embodiments of the method, n is 1 or 2.

In some embodiments of the method, m is 1 or 2.

In some embodiments of the method, n is 1.

In some embodiments of the method, m is 1.

In some embodiments of the method, n and m are the same.

In some embodiments of the method, n and m are different.

In some embodiments of the method, R₁ and R₂ are each independently H, halogen, alkyl, alkenyl, alkynyl, —OH, —O-(alkyl), —CHF₂, —CF₃, —OCHF₂ or —OCF₃, wherein n is 1, 2, 3, 4, 5, or 6.

In some embodiments of the method, R₁ and R₂ are each independently H, halogen, C₁-C₆ alkyl, C₁-C₆ alkenyl, or C₁-C₆ alkynyl.

In some embodiments of the method, R₁ and R₂ are each independently, C₁-C₆ alkyl or C₁-C₆ alkenyl.

In some embodiments of the method, R₁ and R₂ are each independently C₁-C₆ alkyl.

In some embodiments of the method, R₁ and R₂ are C₁₋₅ alkyl.

In some embodiments of the method, R₁ and R₂ are C₁₋₃ alkyl.

In some embodiments of the method, R₁ and R₂ are methyl.

In some embodiments of the method, R₁ and R₂ are ethyl.

In some embodiments of the method, X is alkyl-aryl-thiourea, alkyl-heteroaryl-thiourea, alkyl-cycloalkyl-thiourea, alkenyl-aryl-thiourea, or alkenyl-heteroaryl-thiourea.

In some embodiments of the method, X is alkyl-aryl-thiourea, alkyl-heteroaryl-thiourea, or alkyl-cycloalkyl-thiourea.

In some embodiments of the method, X is alkyl-aryl-thiourea, or alkyl-heteroaryl-thiourea.

In some embodiments of the method, X is alkyl-aryl-thiourea.

In some embodiments of the method, alkyl is C₁₋₆ alkyl.

In some embodiments of the method, alkyl is C₁₋₃ alkyl.

In some embodiments of the method, alkyl is methyl.

In some embodiments of the method, alkyl is ethyl.

In some embodiments of the method, aryl is phenyl, p-toluenyl (4-methylphenyl), naphthyl, tetrahydronaphthyl; indanyl, biphenyl, phenanthryl, anthryl or acenaphthyl.

In some embodiments of the method, aryl is phenyl, p-toluenyl (4-methylphenyl), or naphthyl.

In some embodiments of the method, aryl is phenyl.

In some embodiments of the method, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments of the method, the chemical linker L is an alkyl linker, an alkyne linker, alkynal linker or a polyethylene glycol (PEG) or combinations thereof.

In some embodiments of the method, the chemical linker L is an alkyl or a PEG or combinations thereof.

In some embodiments of the method, the chemical linker L is a PEG.

In some embodiments of the method, the chemical linker L has the following structure:

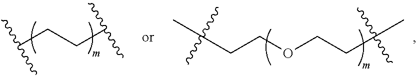

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method, the chemical linker L has the following structure:

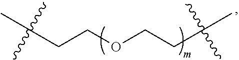

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method of the chemical linker L, m is 1, 3 or 7.

In some embodiments of the method of the chemical linker L, m is 3.

In some embodiments of the method, A is substituted with —OH, —NH₂, halogen, alkyl, —O-alkyl, -alkyl-NH₂, —NH-alkyl, —CHF₂, —CF₃, —OCHF₂, —OCF₃.

In some embodiments the method, A is substituted with —NH₂, -alkyl-NH₂, —NH-alkyl, or alkyl.

In some embodiments of the method, the guest molecule A is substituted or unsubstituted adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments of the method, the guest molecule A is substituted or unsubstituted adamantane, ferrocene, bicyclo[2.2.2]octane, iceane, diamantane, triamantane, isotetramantane, pentamantane, or cyclohexamantane.

In some embodiments of the method, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments of the method, the guest molecule A is substituted or unsubstituted adamantane, diamantane, 4,9-diamino diamantane or ferrocene.

In some embodiments of the method, the guest molecule A is substituted or unsubstituted adamantane or diamantane.

In some embodiments of the method, the guest molecule A is substituted or unsubstituted adamantane.

In some embodiments of the method, the guest molecule A is substituted or unsubstituted diamantane.

In some embodiments of the method, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments of the method, the guest molecule A is substituted with halogen, alkyl, —O-(alkyl), —N-(alkyl).

In some embodiments of the method, the guest molecule A is substituted or unsubstituted ferrocene.

In some embodiments of the method, the guest molecule A is unsubstituted adamantane.

In some embodiments of the method, the guest molecule A is substituted adamantane.

In some embodiments of the method, the guest molecule A is 4,9-diamino diamantane.

In some embodiments of the method, the guest molecule A is unsubstituted ferrocene.

In some embodiments of the method, the guest molecule A is unsubstituted diamantane.

In some embodiments of the method, the guest molecule A is substituted diamantane.

In some embodiments of the method, the substituted diamantane having the following structure:

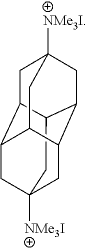

In some embodiments of the method, the guest molecule A is substituted ferrocene.

In some embodiments of the method, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments of the method, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments of the method, the substituted ferrocene is substituted with C₁-C₆ alkyl, -alkyl-N—(C₁-C₆ alkyl).

In some embodiments of the method, the substituted ferrocene is substituted with -alkyl-N—(C₁-C₆ alkyl).

In some embodiments of the method, the substituted ferrocene having the following structure:

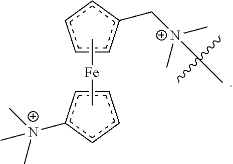

In some embodiments of the method, the metal complex having the structure:

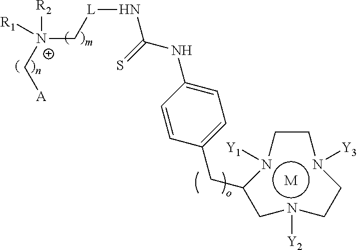

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; or salt or ester thereof.

In some embodiments of the method, the metal complex is other than

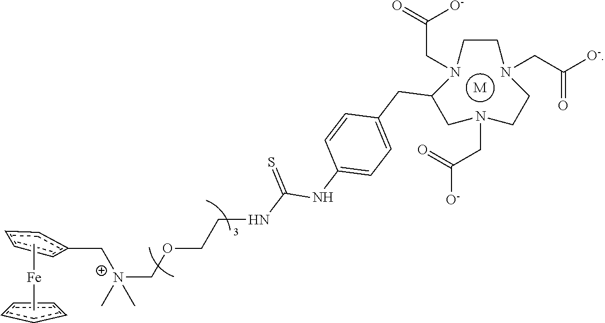

In some embodiments of the method, n and m are each independently 0, 1, 2, 3, 4, 5, or 6.

In some embodiments of the method, n and m are each independently 1, 2, or 3.

In some embodiments of the method, n and m are 1.

In some embodiments of the method, n is 1, 2, or 3.

In some embodiments of the method, m is 1, 2, or 3.

In some embodiments of the method, n is 1 or 2.

In some embodiments of the method, m is 1 or 2.

In some embodiments of the method, n is 1.

In some embodiments of the method, m is 1.

In some embodiments of the method, n and m are the same.

In some embodiments of the method, n and m are different.

In some embodiments of the method, R₁ and R₂ are each independently H, halogen, alkyl, alkenyl, alkynyl, —OH, —O-(alkyl), —CHF₂, —CF₃, —OCHF₂ or —OCF₃, wherein n is 1, 2, 3, 4, 5, or 6.

In some embodiments of the method, R₁ and R₂ are each independently H, halogen, C₁-C₆ alkyl, C₁-C₆ alkenyl, or C₁-C₆ alkynyl.

In some embodiments of the method, R₁ and R₂ are each independently, C₁-C₆ alkyl or C₁-C₆ alkenyl.

In some embodiments of the method, R₁ and R₂ are each independently C₁-C₆ alkyl.

In some embodiments of the method, R₁ and R₂ are C₁₋₅ alkyl.

In some embodiments of the method, R₁ and R₂ are C₁₋₃ alkyl.

In some embodiments of the method, R₁ and R₂ are methyl.

In some embodiments of the method, R₁ and R₂ are ethyl.

In some embodiments of the method, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —OH, —NH₂, alkyl, —O-alkyl, —NH-alkyl, amide, aryl, heteroaryl, or alkyl-CF₃.

In some embodiments of the method, each occurrence of R₄ is independently, —OH, —NH₂, alkyl, —O-alkyl, —NH-alkyl, or amide.

In some embodiments of the method, each occurrence of R₄ is independently, —OH, —NH₂, alkyl, —O-alkyl, or —NH-alkyl.

In some embodiments of the method, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments of the method, Y₁, Y₂, Y₃ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments of the method, Y₁, Y₂, Y₃ are each independently alkyl-CO₂H, or alkyl-CO₂NH₂, or alkyl-N(alkyl-CO₂H)₂.

In some embodiments of the method, at least one of Y₁, Y₂, Y₃ is alkyl-CO₂H.

In some embodiments of the method, at least one of Y₁, Y₂, Y₃ is alkyl-CO₂NH₂.

In some embodiments of the method, at least one of Y₁, Y₂, Y₃ is alkyl-N(alkyl-CO₂H)₂.

In some embodiments of the method, at least one of Y₁, Y₂, Y₃ is —CH₂—CO₂H.

In some embodiments of the method, at least one of Y₁, Y₂, Y₃ is —CH₂—CO₂NH₂.

In some embodiments of the method, at least one of Y₁, Y₂, Y₃ is —CH₂—N(alkyl-CO₂H)₂

In some embodiments of the method, at least two of Y₁, Y₂ and Y₃ are the same.

In some embodiments of the method, at least three of Y₁, Y₂, Y₃ and Y₄ are the same.

In some embodiments of the method, Y₁, Y₂ and Y₃ are the same.

In some embodiments of the method, at least one of Y₁, Y₂, Y₃ and Y₄ is H.

In some embodiments of the method, none of Y₁, Y₂, Y₃ and Y₄ are H.

In some embodiments of the method, Y₁, Y₂, Y₃, Y₄ are each independently —H,

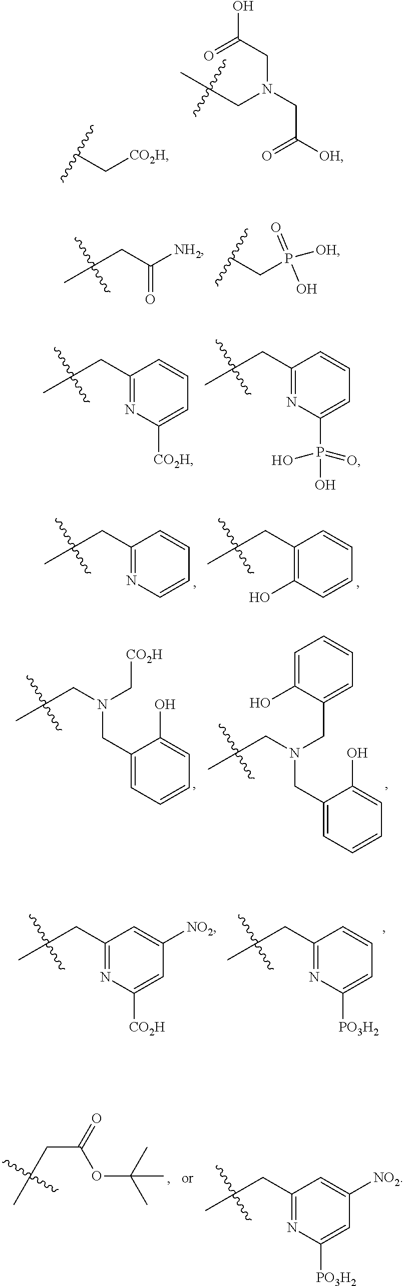

In some embodiments of the method, Y₁, Y₂, Y₃, Y₄ are each independently

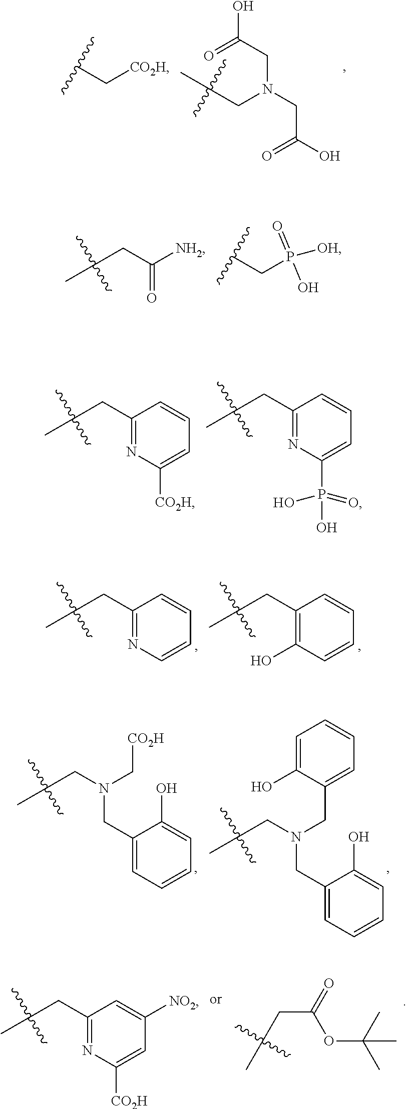

In some embodiments of the method, Y₁, Y₂, Y₃, Y₄ are each independently

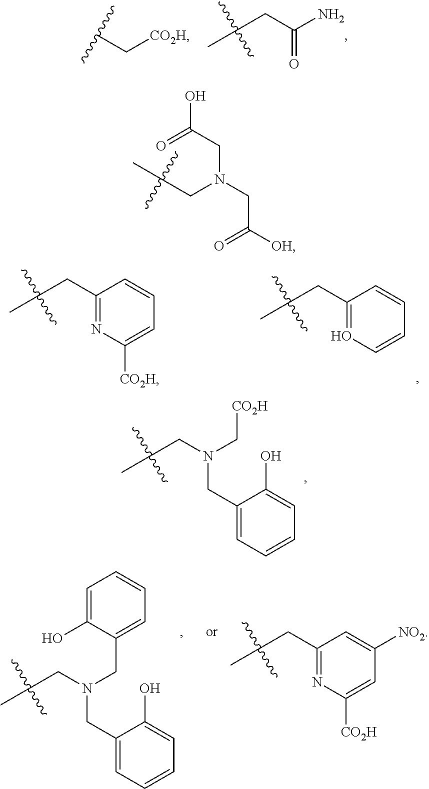

In some embodiments of the method, Y₁, Y₂, Y₃, Y₄ are each independently

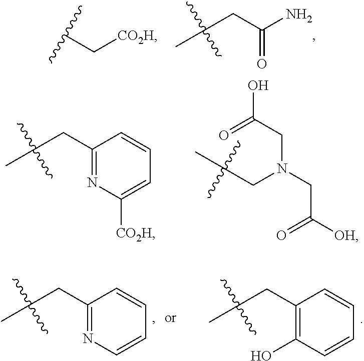

In some embodiments of the method, Y₁, Y₂, Y₃, Y₄ are each independently

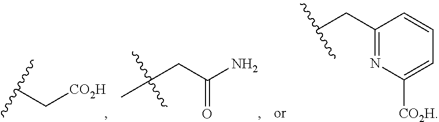 [SMILES: *CC(=O)O ; *CC(N)=O ; *Cc1cccc(C(=O)O)n1]

In some embodiments of the method, Y₁ and Y₃ is

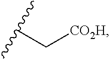 [SMILES: *CC(=O)O]

Y₂ and/or Y₄ is

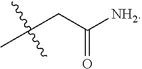 [SMILES: *CC(N)=O]

In some embodiments of the method, X is alkyl-aryl-thiourea, alkyl-heteroaryl-thiourea, alkyl-cycloalkyl-thiourea, alkenyl-aryl-thiourea, or alkenyl-heteroaryl-thiourea.

In some embodiments of the method, X is alkyl-aryl-thiourea, alkyl-heteroaryl-thiourea, or alkyl-cycloalkyl-thiourea.

In some embodiments of the method, X is alkyl-aryl-thiourea, or alkyl-heteroaryl-thiourea.

In some embodiments of the method, X is alkyl-aryl-thiourea.

In some embodiments of the method, alkyl is C₁₋₆ alkyl.

In some embodiments of the method, alkyl is C₁₋₃ alkyl.

In some embodiments of the method, alkyl is methyl.

In some embodiments of the method, alkyl is ethyl.

In some embodiments of the method, aryl is phenyl, p-toluenyl (4-methylphenyl), naphthyl, tetrahydronaphthyl; indanyl, biphenyl, phenanthryl, anthryl or acenaphthyl.

In some embodiments of the method, aryl is phenyl, p-toluenyl (4-methylphenyl), or naphthyl.

In some embodiments of the method, aryl is phenyl.

In some embodiments of the method, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments of the method, the chemical linker L is an alkyl linker, an alkyne linker, alkynal linker or a polyethylene glycol (PEG) or combinations thereof.

In some embodiments of the method, the chemical linker L is an alkyl or a PEG or combinations thereof.

In some embodiments of the method, the chemical linker L is a PEG.

In some embodiments of the method, the chemical linker L has the following structure:

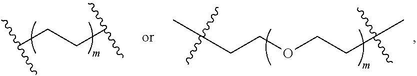

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method, the chemical linker L has the following structure:

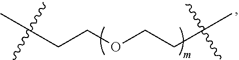

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method of chemical linker L, m is 1, 3 or 7.

In some embodiments of the method of chemical linker L, m is 3.

In some embodiments of the method, A is substituted with —OH, —NH₂, halogen, alkyl, —O-alkyl, -alkyl-NH₂, —NH-alkyl, —CHF₂, —CF₃, —OCHF₂, —OCF₃.

In some embodiments the method, A is substituted with —NH₂, -alkyl-NH₂, —NH-alkyl, or alkyl.

In some embodiments of the method, the guest molecule A is substituted or unsubstituted adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments of the method, the guest molecule A is substituted or unsubstituted adamantane, ferrocene, bicyclo[2.2.2]octane, iceane, diamantane, triamantane, isotetramantane, pentamantane, or cyclohexamantane.

In some embodiments of the method, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments of the method, the guest molecule A is substituted or unsubstituted adamantane, diamantane, 4,9-diamino diamantane or ferrocene.

In some embodiments of the method, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments the method, the guest molecule A is substituted with halogen, alkyl, —O-(alkyl), —N-(alkyl).

In some embodiments of the method, the guest molecule A is substituted or unsubstituted adamantane or diamantane.

In some embodiments of the method, the guest molecule A is substituted or unsubstituted adamantane.

In some embodiments of the method, the guest molecule A is substituted or unsubstituted diamantane.

In some embodiments of the method, the guest molecule A is substituted or unsubstituted ferrocene.

In some embodiments of the method, the guest molecule A is unsubstituted adamantane.

In some embodiments of the method, the guest molecule A is substituted adamantane.

In some embodiments of the method, the guest molecule A is 4,9-diamino diamantane.

In some embodiments of the method, the guest molecule A is unsubstituted ferrocene.

In some embodiments of the method, the guest molecule A is unsubstituted diamantane.

In some embodiments of the method, the guest molecule A is substituted diamantane.

In some embodiments of the method, the substituted diamantane having the following structure:

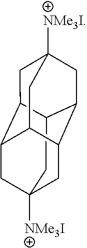

In some embodiments of the method, the guest molecule A is substituted ferrocene.

In some embodiments of the method, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments of the method, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments of the method, the substituted ferrocene is substituted with C₁-C₆ alkyl, -alkyl-N—(C₁-C₆ alkyl).

In some embodiments of the method, the substituted ferrocene is substituted with -alkyl-N—(C₁-C₆ alkyl).

In some embodiments of the method, the substituted ferrocene having the following structure:

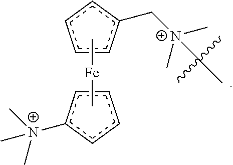

In some embodiments of the method, the metal complex having the structure:

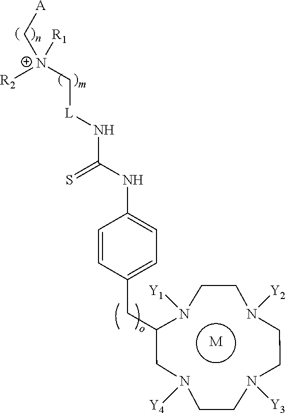

wherein M is the metal;
wherein Y₁, Y₂, Y₃, Y₄ are each, independently, —H, alkyl-N—(CO₂R₄)₂, alkyl-N-(alkyl-CO₂R₄)₂ alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments of the method, the metal complex having the structure:

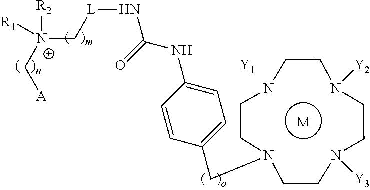

wherein M is the metal;
wherein Y₁, Y₂, Y₃ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
   wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃;
wherein L is a chemical linker; and
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

In some embodiments of the method, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —OH, —NH₂, alkyl, —O-alkyl, —NH-alkyl, amide, aryl, heteroaryl, or alkyl-CF₃.

In some embodiments of the method, each occurrence of R₄ is independently, —OH, —NH₂, alkyl, —O-alkyl, —NH-alkyl, amide.

In some embodiments of the method, each occurrence of R₄ is independently, —OH, —NH₂, alkyl, —O-alkyl, —NH-alkyl.

In some embodiments of the method, R₄ is —OH.

In some embodiments of the method, R₄ is —NH₂.

In some embodiments of the method, n and m are each independently 0, 1, 2, 3, 4, 5, or 6.

In some embodiments of the method, n and m are each independently 1, 2, or 3.

In some embodiments of the method, n and m are 1.

In some embodiments of the method, n is 1, 2, or 3.

In some embodiments of the method, m is 1, 2, or 3.

In some embodiments of the method, n is 1 or 2.

In some embodiments of the method, m is 1 or 2.

In some embodiments of the method, n is 1.

In some embodiments of the method, m is 1.

In some embodiments of the method, n and m are the same.

In some embodiments of the method, n and m are different.

In some embodiments of the method, R₁ and R₂ are each independently H, halogen, alkyl, alkenyl, alkynyl, —OH, —O-(alkyl), —CHF₂, —CF₃, —OCHF₂ or —OCF₃, wherein n is 1, 2, 3, 4, 5, or 6.

In some embodiments of the method, R₁ and R₂ are each independently H, halogen, C₁-C₆ alkyl, C₁-C₆ alkenyl, or C₁-C₆ alkynyl.

In some embodiments of the method, R₁ and R₂ are each independently, C₁-C₆ alkyl or C₁-C₆ alkenyl.

In some embodiments of the method, R₁ and R₂ are each independently C₁-C₆ alkyl.

In some embodiments of the method, R₁ and R₂ are C₁₋₅ alkyl.

In some embodiments of the method, R₁ and R₂ are C₁₋₃ alkyl.

In some embodiments of the method, R₁ and R₂ are methyl.

In some embodiments of the method, R₁ and R₂ are ethyl.

In some embodiments of the method, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-Nalkylheteroaryl-OH)(alkyl-CO₂H), or alkylheteroaryl-P(O)(OH)₂.

In some embodiments of the method, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂.

In some embodiments of the method, Y₁, Y₂, Y₃, Y₄ are each independently alkyl-CO₂H, or alkyl-CO₂NH₂, or alkyl-N(alkyl-CO₂H)₂.

In some embodiments of the method, at least one of Y₁, Y₂, Y₃, Y₄ is alkyl-CO₂H.

In some embodiments of the method, at least one of Y₁, Y₂, Y₃, Y₄ is alkyl-CO₂NH₂.

In some embodiments of the method, at least one of Y₁, Y₂, Y₃, Y₄ is alkyl-N(alkyl-CO₂H)₂.

In some embodiments of the method, at least one of Y₁, Y₂, Y₃, Y₄ is —CH₂—CO₂H.

In some embodiments of the method, at least one of Y₁, Y₂, Y₃, Y₄ is —CH₂—CO₂NH₂.

In some embodiments of the method, at least one of Y₁, Y₂, Y₃, Y₄ is —CH₂—N(alkyl-CO₂H)₂

In some embodiments of the method, at least two of Y₁, Y₂, Y₃, Y₄ are the same.

In some embodiments of the method, at least three of Y₁, Y₂, Y₃, Y₄ are the same.

In some embodiments of the method, Y₁, Y₂, Y₃, Y₄ are the same.

In some embodiments of the method, at least one of Y₁, Y₂, Y₃ and Y₄ is H.

In some embodiments of the method, none of Y₁, Y₂, Y₃ and Y₄ are H.

In some embodiments of the method, Y₁, Y₂, Y₃, Y₄ are each independently —H,

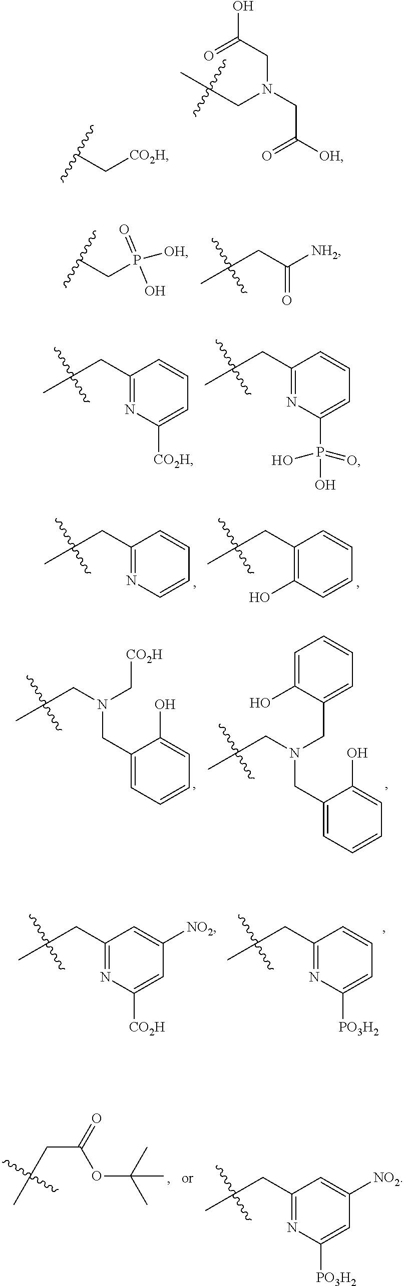

In some embodiments of the method, Y₁, Y₂, Y₃, Y₄ are each independently

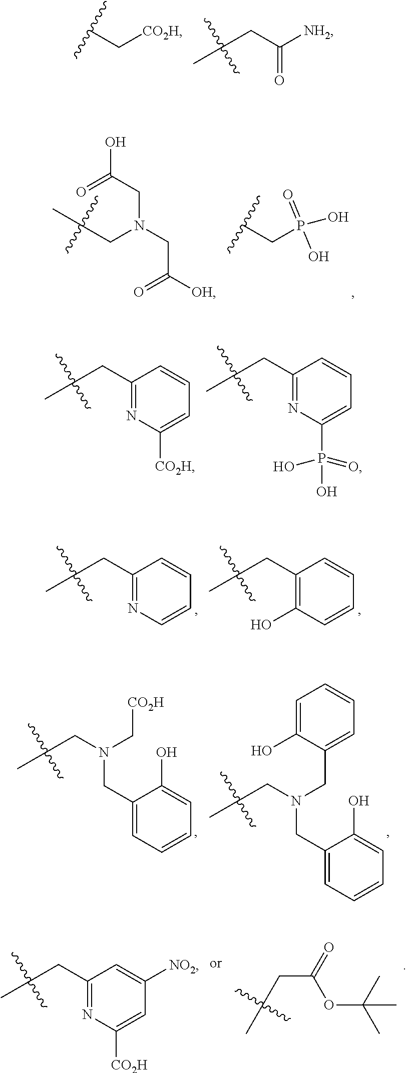

In some embodiments of the method, Y₁, Y₂, Y₃, Y₄ are each independently

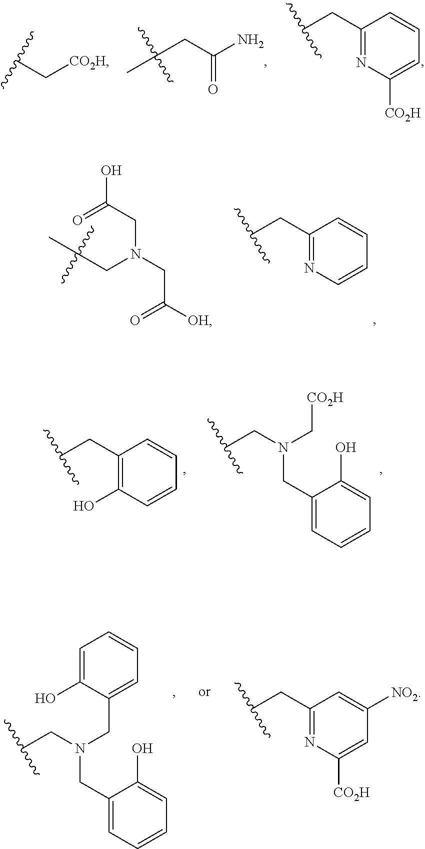

In some embodiments of the method, Y₁, Y₂, Y₃, Y₄ are each independently

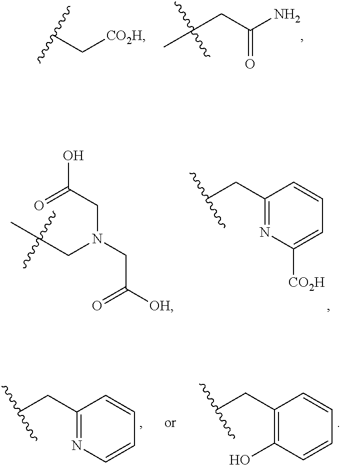 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]

In some embodiments of the method, Y₁, Y₂, Y₃, Y₄ are each independently

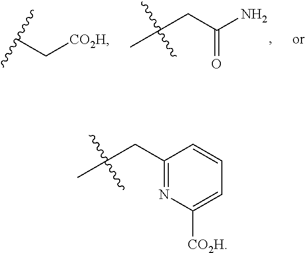 [SMILES: *CC(=O)O ; *CC(N)=O ; *Cc1cccc(C(=O)O)n1]

In some embodiments of the method, Y₁ and Y₃ is

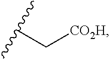 [SMILES: *CC(=O)O]

Y₂ and/or Y₄ is

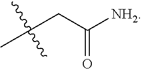 [SMILES: *CC(N)=O]

In some embodiments of the method, X is alkyl-aryl-thiourea, alkyl-heteroaryl-thiourea, alkyl-cycloalkyl-thiourea, alkenyl-aryl-thiourea, or alkenyl-heteroaryl-thiourea.

In some embodiments of the method, X is alkyl-aryl-thiourea, alkyl-heteroaryl-thiourea, or alkyl-cycloalkyl-thiourea.

In some embodiments of the method, X is alkyl-aryl-thiourea, or alkyl-heteroaryl-thiourea.

In some embodiments of the method, X is alkyl-aryl-thiourea.

In some embodiments of the method, alkyl is C₁₋₆ alkyl.

In some embodiments of the method, alkyl is C₁₋₃ alkyl.

In some embodiments of the method, alkyl is methyl.

In some embodiments of the method, alkyl is ethyl.

In some embodiments of the method, aryl is phenyl, p-toluenyl (4-methylphenyl), naphthyl, tetrahydronaphthyl; indanyl, biphenyl, phenanthryl, anthryl or acenaphthyl.

In some embodiments of the method, aryl is phenyl, p-toluenyl (4-methylphenyl), or naphthyl.

In some embodiments of the method, aryl is phenyl.

In some embodiments of the method, the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof.

In some embodiments of the method, the chemical linker L is an alkyl linker, an alkyne linker, alkynal linker or a polyethylene glycol (PEG) or combinations thereof.

In some embodiments of the method, the chemical linker L is an alkyl or a PEG or combinations thereof.

In some embodiments of the method, the chemical linker L is a PEG.

In some embodiments of the method, the chemical linker L has the following structure:

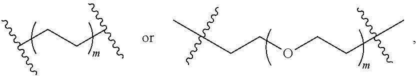

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method, the chemical linker L has the following structure:

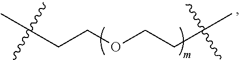

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method of chemical linker L, m is 1, 3 or 7.

In some embodiments of the method chemical linker L, m is 3.

In some embodiments of the method, A is substituted with —OH, —NH₂, halogen, alkyl, —O-alkyl, -alkyl-NH₂, —NH-alkyl, —CHF₂, —CF₃, —OCHF₂, —OCF₃.

In some embodiments the method, A is substituted with —NH₂, -alkyl-NH₂, —NH-alkyl, or alkyl.

In some embodiments of the method, the guest molecule A is substituted or unsubstituted adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments of the method, A is substituted with —OH, —NH₂, halogen, alkyl, —O-alkyl, -alkyl-NH₂, —NH-alkyl, —CHF₂, —CF₃, —OCHF₂, —OCF₃.

In some embodiments of the method, A is substituted with —NH₂, -alkyl-NH₂, —NH-alkyl, or alkyl.

In some embodiments of the method, the guest molecule A is substituted or unsubstituted adamantane, ferrocene, bicyclo[2.2.2]octane, iceane, diamantane, triamantane, isotetramantane, pentamantane, or cyclohexamantane.

In some embodiments of the method, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments of the method, the guest molecule A is substituted or unsubstituted adamantane, diamantane, 4,9-diamino diamantane or ferrocene.

In some embodiments of the method, the guest molecule A is substituted or unsubstituted adamantane or diamantane.

In some embodiments of the method, the guest molecule A is substituted or unsubstituted adamantane.

In some embodiments of the method, the guest molecule A is substituted or unsubstituted diamantane.

In some embodiments of the method, the guest molecule A is substituted or unsubstituted ferrocene.

In some embodiments of the method, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments of the method, the guest molecule A is substituted with halogen, alkyl, —O-(alkyl), —N-(alkyl).

In some embodiments of the method, the guest molecule A is unsubstituted adamantane.

In some embodiments of the method, the guest molecule A is substituted adamantane.

In some embodiments of the method, the guest molecule A is 4,9-diamino diamantane.

In some embodiments of the method, the guest molecule A is unsubstituted ferrocene.

In some embodiments of the method, the guest molecule A is unsubstituted diamantane.

In some embodiments of the method, the guest molecule A is substituted diamantane.

In some embodiments of the method, the substituted diamantane having the following structure:

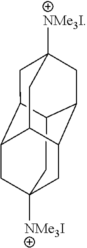

In some embodiments of the method, the guest molecule A is substituted ferrocene.

In some embodiments of the method, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments of the method, the substituted ferrocene is substituted with C₁-C₆ alkyl, -alkyl-N—(C₁-C₆ alkyl), —OH, —O—(C₁-C₆ alkyl), —NH—(C₁-C₆ alkyl).

In some embodiments of the method, the substituted ferrocene is substituted with C₁-C₆ alkyl, -alkyl-N—(C₁-C₆ alkyl).

In some embodiments of the method, the substituted ferrocene is substituted with -alkyl-N—(C₁-C₆ alkyl).

In some embodiments of the method, the substituted ferrocene having the following structure:

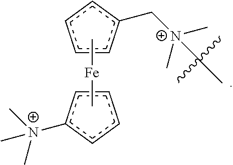

In some embodiments of the method, the metal complex having the structure:

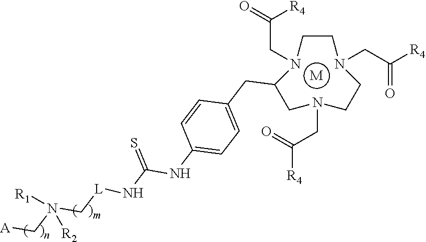

wherein M is the metal;
wherein L is a chemical linker;
wherein n and m are each independently 1, 2, or 3;
wherein A is a guest molecule which is substituted and unsubstituted adamantane, ferrocene, diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments of the method, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —OH, —NH₂, alkyl, —O-alkyl, —NH-alkyl, amide, aryl, heteroaryl, or alkyl-CF₃.

In some embodiments of the method, each occurrence of R₄ is independently, —OH, —NH₂, alkyl, —O-alkyl, —NH-alkyl, or amide.

In some embodiments of the method, each occurrence of R₄ is independently, —OH, —NH₂, alkyl, —O-alkyl, or —NH-alkyl.

In some embodiments of the method, R₄ is —OH.

In some embodiments of the method, R₄ is —NH₂.

In some embodiments of the method, the metal complex having the structure:

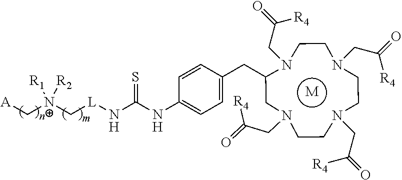

wherein M is the metal;
wherein L is a chemical linker;
wherein n and m are each independently 1, 2, or 3;
wherein A is a guest molecule which is substituted and unsubstituted adamantane, ferrocene, diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments of the method, the metal complex having the structure:

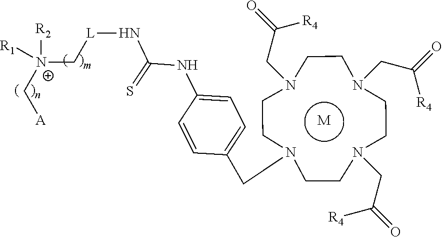

wherein M is the metal;
wherein L is a chemical linker;
wherein n and m are each independently 1, 2, or 3;
wherein A is a guest molecule which is substituted and unsubstituted adamantane, ferrocene, diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments of the method, each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —H, —OH, —NH₂, halogen, alkyl, —O-alkyl, —NH-alkyl, amide, alkenyl, alkynyl, alkyl-aryl, alkyl-heteroaryl, aryl, heteroaryl, alkyl-CF₃, or —Si(alkyl)₃.

In some embodiments of the method, each occurrence of R₄ is independently, —OH, —NH₂, alkyl, —O-alkyl, —NH-alkyl, amide, aryl, heteroaryl, or alkyl-CF₃.

In some embodiments of the method, each occurrence of R₄ is independently, —OH, —NH₂, alkyl, —O-alkyl, —NH-alkyl, or amide.

In some embodiments of the method, each occurrence of R₄ is independently, —OH, —NH₂, alkyl, —O-alkyl, or —NH-alkyl.

In some embodiments of the method, R₄ is —OH.

In some embodiments of the method, R₄ is —NH₂.

In some embodiments of the method, alkyl is C₁₋₆ alkyl.

In some embodiments of the method, alkyl is C₁₋₃ alkyl.

In some embodiments of the method, alkyl is methyl.

In some embodiments of the method, alkyl is ethyl.

In some embodiments of the method, aryl is phenyl, p-toluenyl (4-methylphenyl), naphthyl, tetrahydronaphthyl; indanyl, biphenyl, phenanthryl, anthryl or acenaphthyl.

In some embodiments of the method, the chemical linker L is an alkyl, alkenyl, alkynyl, alkylether, alkylthioether, alkylamino, alkylamido, alkylester, alkylaryl, 125lkylheteroaryl, polyethylene glycol (PEG), aryl, heteroaryl, a natural amino acid, an unnatural amino acid, a disulfide or thioether containing linker or combinations thereof.

In some embodiments of the method, the chemical linker L is an alkyl linker, an alkyne linker, alkynal linker or a polyethylene glycol (PEG) or combinations thereof.

In some embodiments of the method, the chemical linker L is an alkyl or a PEG or combinations thereof.

In some embodiments of the method, the chemical linker L is a PEG.

In some embodiments of the method, the chemical linker L has the following structure:

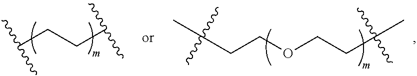

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method, the chemical linker L has the following structure:

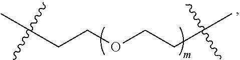

wherein m is 1, 2, 3, 4, 5, 6, 7, 8, 9, or 10; preferably m is 1, 2, 3, 4, 5, 6, or 7; more preferably, m is 1, 3, or 7.

In some embodiments of the method of chemical linker L, m is 1, 3 or 7.

In some embodiments of the method of chemical linker L, m is 3.

In some embodiments of the method, A is substituted with —OH, —NH₂, halogen, alkyl, —O-alkyl, -alkyl-NH₂, —NH-alkyl, —CHF₂, —CF₃, —OCHF₂, —OCF₃.

In some embodiments, of the method, A is substituted with —NH₂, -alkyl-NH₂, —NH-alkyl, or alkyl.

In some embodiments of the method, the guest molecule A is substituted or unsubstituted adamantane, diamantane, ferrocene, bicyclo[2.2.2]octane, buckminsterfullerene (C60), iceane, triamantane, isotetramantane, ferrocene-modified peracetic acid, pentamantane, or cyclohexamantane.

In some embodiments of the method, the guest molecule A is substituted or unsubstituted adamantane, 4,9-diamino diamantane, ferrocene, bicyclo[2.2.2]octane, iceane, diamantane, triamantane, isotetramantane, pentamantane, or cyclohexamantane.

In some embodiments of the method, the guest molecule A is substituted or unsubstituted adamantane, diamantane, 4,9-diamino diamantane or ferrocene.

In some embodiments of the method, the guest molecule A is substituted or unsubstituted adamantane or diamantane.

In some embodiments of the method, A is substituted with —OH, —NH₂, halogen, alkyl, —O-alkyl, -alkyl-NH₂, —NH-alkyl, —CHF₂, —CF₃, —OCHF₂, —OCF₃.

In some embodiments of the method, A is substituted with —NH₂, -alkyl-NH₂, —NH-alkyl, or alkyl.

In some embodiments of the method, the guest molecule A is substituted with halogen, alkyl, alkenyl, alkynyl, —OH, —O-(alkyl), —N-(alkyl), —CHF₂, —CF₃, —OCHF₂ or —OCF₃.

In some embodiments of the method, the guest molecule A is substituted with halogen, alkyl, -0-(alkyl), or —N-(alkyl).

In some embodiments of the method, the guest molecule A is substituted or unsubstituted adamantane.

In some embodiments of the method, the guest molecule A is substituted or unsubstituted diamantane.

In some embodiments of the method, the guest molecule A is substituted or unsubstituted ferrocene.

In some embodiments of the method, the guest molecule A is unsubstituted adamantane.

In some embodiments of the method, the guest molecule A is substituted adamantane.

In some embodiments of the method, the guest molecule A is 4,9-diamino diamantane.

In some embodiments of the method, the guest molecule A is unsubstituted ferrocene.

In some embodiments of the method, the guest molecule A is unsubstituted diamantane.

In some embodiments of the method, the guest molecule A is substituted diamantane.

In some embodiments of the method, the substituted diamantane having the following structure:

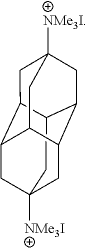

In some embodiments of the method, the guest molecule A is substituted ferrocene.

In some embodiments of the method, the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃.

In some embodiments of the method, the substituted ferrocene is substituted with C₁-C₆ alkyl, -alkyl-N—(C₁-C₆ alkyl), —OH, —O—(C₁-C₆ alkyl), or —NH—(C₁-C₆ alkyl).

In some embodiments of the method, the substituted ferrocene is substituted with C₁-C₆ alkyl, or -alkyl-N—(C₁-C₆ alkyl).

In some embodiments of the method, the substituted ferrocene having the following structure:

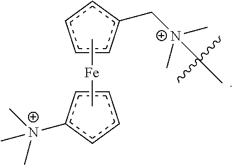

In some embodiments of the method, the metal complex having the structure:

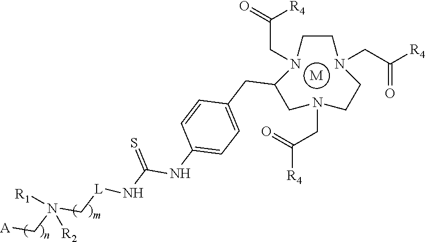

wherein \textcircled{M} is the metal;
wherein L is alkyl, alkenyl, alkynyl, alkylether, alkylthioether, alkylamino, alkylamido, alkylester, alkylaryl, alklyheteroaryl, or polyethylene glycol (PEG);
wherein n and m are each independently 1, 2, or 3;
wherein A is a guest molecule which is substituted or unsubstituted adamantane, ferrocene, diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments of the method, the metal complex having the structure:

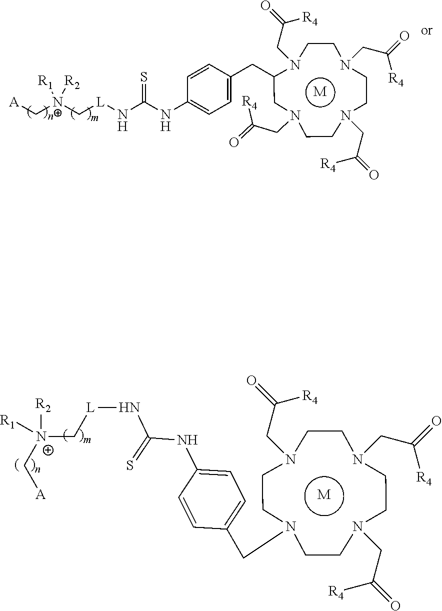

wherein \textcircled{M} is the metal;
wherein L is alkyl, alkenyl, alkynyl, alkylether, alkylthioether, alkylamino, alkylamido, alkylester, alkylaryl, alklyheteroaryl, or polyethylene glycol (PEG);
wherein n and m are each independently 1, 2, or 3;
wherein A is a guest molecule which is substituted or unsubstituted adamantane, ferrocene, diamantane, bicyclo[2.2.2]octane, iceane, triamantane, isotetramantane, pentamantane cyclohexamantane, super-adamantane, 1,3,5,7-tetramethyl-1,3,5,7-tetrasilaadamantane; and
wherein each occurrence of R₄ is independently, —H, —OH, —NH₂, halogen, alkyl, —O-alkyl, —NH-alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂.

In some embodiments of the method, the metal complex having the structure:

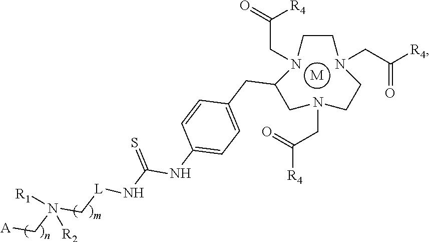

wherein \textcircled{M} is the metal;
wherein L is a chemical linker;
wherein n and m are each independently 1, 2, or 3;
wherein A is a guest molecule which is adamantane, ferrocene, or diamantane,
wherein R₄ is —OH, —NH₂, —O—(C₁-C₆ alkyl), or NH—(C₁-C₆ alkyl), more preferably R₄ is —OH or —NH₂.

In some embodiments of the method, the metal complex having the structure:

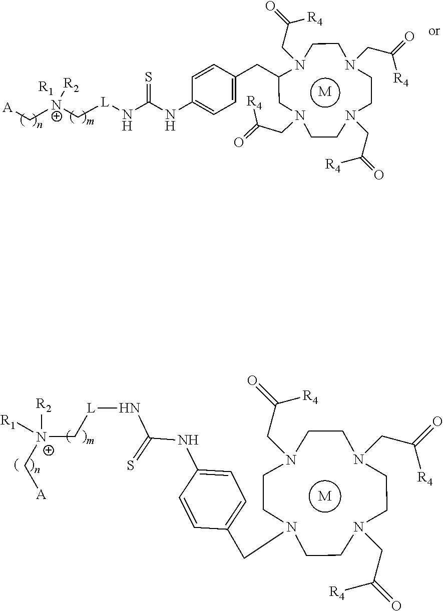

wherein \textcircled{M} is the metal;
wherein L is a chemical linker;
wherein n and m are each independently 1, 2, or 3;
wherein A is a guest molecule which is adamantane, ferrocene, or diamantane; and
wherein R₄ is —OH, —O—(C₁-C₆ alkyl), or NH—(C₁-C₆ alkyl), more preferably R₄ is —OH.

In some embodiments of the method, the metal complex having the structure:

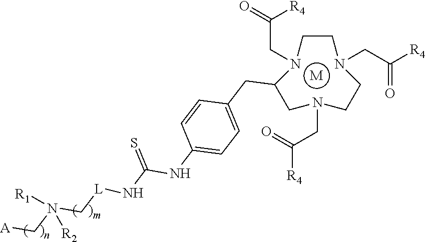

wherein \textcircled{M} is the metal;
wherein L is alkyl, alkenyl, alkynyl, alkylether, or polyethylene glycol (PEG);
wherein n and m are each independently 1, 2, or 3;
wherein A is a guest molecule which is adamantane, ferrocene, or diamantane; and
wherein R₄ is —OH, —NH₂, —O—(C₁-C₆ alkyl), or NH—(C₁-C₆ alkyl), more preferably R₄ is —OH or —NH₂.

In some embodiments of the method, the metal complex having the structure:

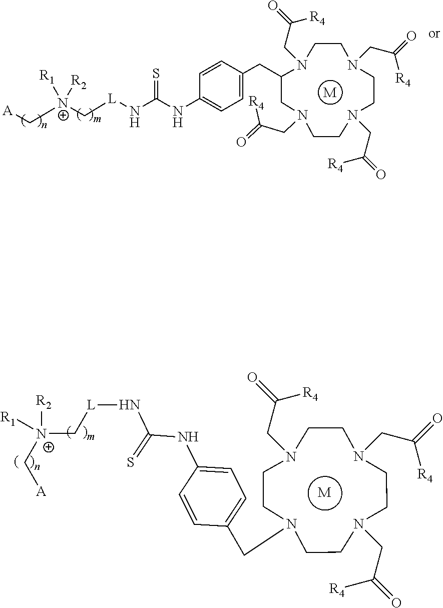

wherein \textcircled{M} is the metal;
wherein L is alkyl, alkenyl, alkynyl, alkylether, or polyethylene glycol (PEG);
wherein n and m are each independently 1, 2, or 3;
wherein A is a guest molecule which is adamantane, ferrocene, or diamantane; and
wherein R₄ is —OH, —NH₂, —O—(C₁-C₆ alkyl), or NH—(C₁-C₆ alkyl), more preferably R₄ is —OH or —NH₂.

In some embodiments of the method, the metal complex having the structure:

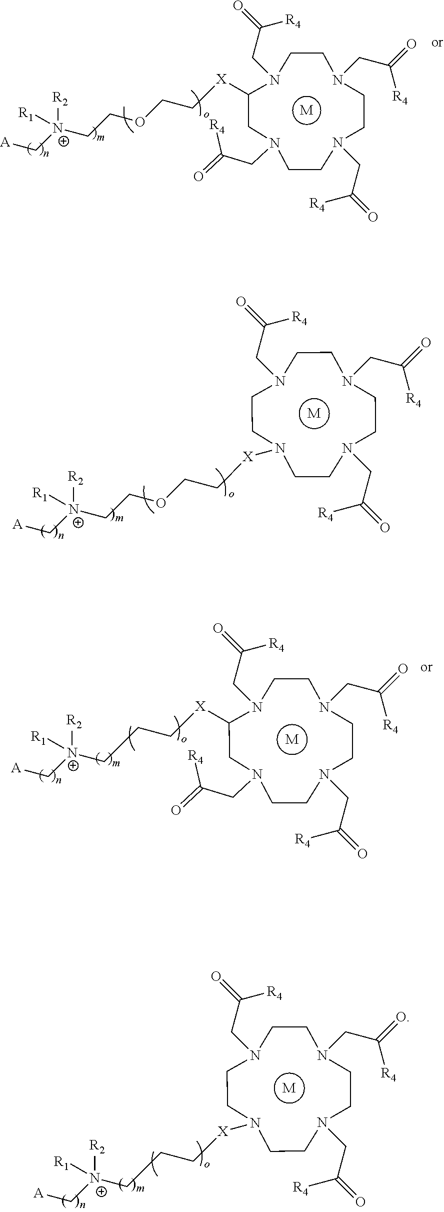

In some embodiments of the method, the metal complex having the structure:

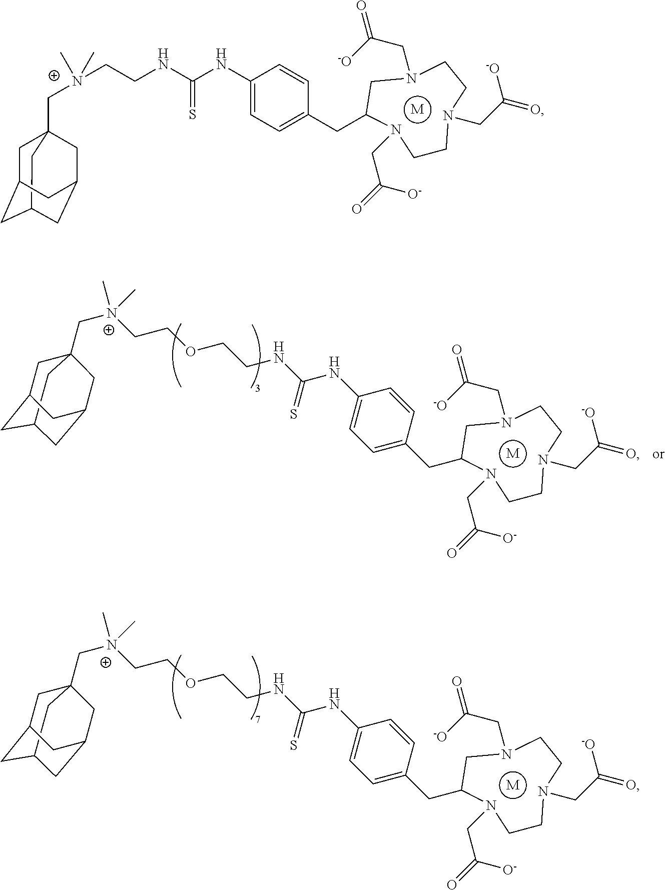

wherein \textcircled{M} is the metal.

In some embodiments of the method, the metal is Copper-62 (⁶²Cu), Copper-64 (⁶⁴Cu), Copper-67 (⁶⁷Cu), Gallium-68 (⁶⁸Ga) Scandium-44 (⁴⁴Sc), Scandium-47 (⁴⁷Sc), Scandium-43 (⁴³Sc), Lead-203 (²⁰³Pb), Lead-212 (²¹²Pb), Lanthanum-132 (¹³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Th)

In some embodiments of the method, the metal is Copper-62 (⁶²Cu), Copper-64 (⁶⁴Cu), Copper-67 (⁶⁷Cu), Scandium-44 (⁴⁴Sc), Scandium-47 (⁴⁷Sc), or Scandium-43 (⁴³Sc).

In some embodiments of the method, the metal is Copper-64 (⁶⁴Cu).

The present invention provides a method of detecting cells in a subject comprises administering an effective amount of a pharmaceutical composition comprising the metal complex described in the invention and a marker attached to a host molecule.

In some embodiments of the method, the marker is a biological marker.

In some embodiments of the method, the marker is a tumor marker or a cancer marker.

In some embodiments of the method, the tumor marker is prostate-specific antigen (PSA), prostatic acid phosphatase (PAP), cancer antigen 125 (CA 125), carcinoembryonic antigen (CEA), alpha-fetoprotein (AFP), human chorionic gonadotropin (HCG), cancer antigen 19-9 (CA 19-9), cancer antigen 15-3 (CA 15-3), cancer antigen 27-29 (CA 27-29), lactate dehydrogenase (LDH), or neuron-specific enolase (NSE).

In some embodiments of the method, the tumor marker is prostate-specific antigen (PSA), cancer antigen 125 (CA 125), carcinoembryonic antigen (CEA), cancer antigen 19-9 (CA 19-9), cancer antigen 15-3 (CA 15-3), or cancer antigen 27-29 (CA 27-29).

In some embodiments of the method, the tumor marker is prostate-specific antigen (PSA) or carcinoembryonic antigen (CEA).

In some embodiments of the method, the tumor marker is carcinoembryonic antigen (CEA).

In some embodiments of the method, the host molecule comprises cucurbit[5]uril, cucurbit[6]uril, cucurbit[7]uril, cucurbit[8]uril, cucurbit[10]uril, cucurbit[14]uril, cyclodextrin, calix-[5]-arenes.

In some embodiments of the method, the host molecule comprises cucurbit[5]uril, cucurbit[6]uril, cucurbit[7]uril, cucurbit[8]uril, or cucurbit[10]uril.

In some embodiments of the method, the host molecule comprises cucurbit[5]uril, cucurbit[6]uril, cucurbit[7]uril, or cucurbit[8]uril.

In some embodiments of the method, the host molecule comprises cucurbit[7]uril, or cucurbit[8]uril.

In some embodiments of the method, the host molecule is cucurbit[7]uril.

In some embodiments of the method, the interaction between the host and the guest molecule is a non-covalent interaction.

In some embodiments of the method, the non-covalent interaction is ion-ion interaction, ion-dipole interaction, dipole-dipole interaction, hydrogen bonding, cation-π interaction, π-π interaction, van der Waals interaction or hydrophobic interaction.

In some embodiments of the method, the non-covalent interaction is ion-ion interaction, or van der Waals interaction.

In some embodiments of the method, the metal complex described in the invention and the host molecule described in the invention form a high affinity host-guest complex.

The present invention provides a method of detecting cells in a subject comprises administering an effective amount of the modified marker with a host molecule described in the invention to the subject who contains a guest molecule.

The present invention provides a method of detecting cells in a subject comprising administering an effective amount of the pharmaceutical composition described in the invention to the subject and imaging the subject with a molecular imaging device to detect the composition in the subject.

The present invention provides a method of imaging cells in a subject comprising:
1) administering to the subject an effective amount of the composition described in the invention, wherein the composition specifically accumulates at the cells in the subject;
2) detecting in the subject the location of the composition; and
3) obtaining an image of the cells in the subject based on the location of the composition in the subject.

The present invention provides a method of detecting the presence of cells in a subject which comprises determining if an amount of the composition described in the invention is present in the subject at a period of time after administration of the composition to the subject, thereby detecting the presence of the cells based on the amount of the composition determined to be present in the subject.

In some embodiments of the method, the metal complex and the host molecule are applied concurrently, or wherein the host molecule is applied first, and the metal complex is applied after a period of time.

In some embodiments of the method, the period of time is 24 hours, 48 hours, 72, hours, 96 hours, 120 hours, or 144 hours.

In some embodiments of the method, the period of time is 72 hours.

In some embodiments of the method, the cells are cancer cells or tumor cells.

In some embodiments of the method, the cancer cells or tumor cells have elevated levels of proteins or antigens, or both.

In some embodiments of the method, the cancer is lung cancer, breast cancer, prostate cancer, cervical cancer, pancreatic cancer, colon cancer, ovarian cancer, stomach cancer, esophagus cancer, skin cancer, heart cancer, liver cancer, bronchial cancer, testicular cancer, kidney cancer, bladder cancer, spleen cancer, thymus cancer, thyroid cancer, brain cancer, or gall bladder cancer.

In some embodiments of the method, the cancer is pancreatic cancer.

In some embodiments of the method, the tumor is bone tumor, brain tumor, malignant soft tissue tumor, organ tumor, ovarian germ cell tumor, gland tumor, lymphatic tumor, or skin tumor.

In some embodiments of the method, the subject is a mammal.

In some embodiments of the method, the subject is a human.

In some embodiments of the method, the molecular imaging device is a PET imaging device.

The present invention provides a use of an effective amount of metal complex described in the invention to detect cells within a subject, wherein the subject contains a host molecule.

The present invention provides a use of an effective amount of a marker attached to a host molecule described in the invention to detect cells in a subject, wherein the subject contains a guest molecule.

The present invention provides a use of an effective amount of the composition described in the invention to image a subject with a molecular imaging device to detect cells in a subject.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

Exemplary functional groups of Y₁, Y₂, Y₃, Y₄ are described in U.S. Patent Application Publication No. 2021/0276971 A1, International Application No. US/2022/078389, and International Application No. PCT/US2023/064637, the contents of which are hereby incorporated by reference.

Compounds and metal complexes disclosed and tested in U.S. Patent Application Publication No. 2021/0276971 A1, International Application No. US/2022/078389, and International Application No. PCT/US2023/064637 can be used to attach to a guest molecule as disclosed in the subject application. The contents of U.S. Patent Application Publication No. 2021/0276971 A1, International Application No. US/2022/078389, and International Application No. PCT/US2023/064637 are hereby incorporated by reference.

Compounds and metal complexes disclosed and tested in U.S. Patent Application Publication No. 2021/0276971 A1, International Application No. US/2022/078389, and International Application No. PCT/US2023/064637 can be used to attach to a guest molecule as disclosed in the subject application for PET imaging in a subject. The contents of U.S. Patent Application Publication No. 2021/0276971 A1, International Application No. US/2022/078389, and International Application No. PCT/US2023/064637 are hereby incorporated by reference.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, the term “biological marker” refers to a broad subcategory of medical signs—that is, objective indications of medical state observed from outside the patient—which can be measured accurately and reproducibly. Medical signs stand in contrast to medical symptoms, which are limited to those indications of health or illness perceived by patients themselves. In 1998, the National Institutes of Health Biomarkers Definitions Working Group defined a biomarker as “a characteristic that is objectively measured and evaluated as an indicator of normal biological processes, pathogenic processes, or pharmacologic responses to a therapeutic intervention.” A joint venture on chemical safety, the International Programme on Chemical Safety, led by the World Health Organization (WHO) and in coordination with the United Nations and the International Labor Organization, has defined a biomarker as “any substance, structure, or process that can be measured in the body or its products and influence or predict the incidence of outcome or disease”. An even broader definition takes into account not just incidence and outcome of disease, but also the effects of treatments, interventions, and even unintended environmental exposure, such as to chemicals or nutrients. In their report on the validity of biomarkers in environment risk assessment, the WHO has stated that a true definition of biomarkers includes “almost any measurement reflecting an interaction between a biological system and a potential hazard, which may be chemical, physical, or biological. The measured response may be functional and physiological, biochemical at the cellular level, or a molecular interaction.” Examples of biomarkers include everything from pulse and blood pressure through basic chemistries to more complex laboratory tests of blood and other tissues.

As used herein, the term “Guest-Host” refers to host guest interactions involving two molecules or materials that can form complexes through unique structural relationships and noncovalent binding. Also referred to as molecular recognition, this type of interaction is widely found in biorecognition processes, such as enzyme-inhibitor and antigen-antibody interactions.

As used herein the term “Prostate-specific antigen (PSA)” is always present in low concentrations in the blood of adult males. An elevated PSA level in the blood may indicate prostate cancer, but other conditions such as benign prostatic hyperplasia (BPH) and prostatitis can also raise PSA levels. PSA levels are used to evaluate how a patient has responded to treatment and to check for tumor recurrence.

As used herein the term “Prostatic acid phosphatase (PAP)” originates in the prostate and is normally present in small amounts in the blood. In addition to prostate cancer, elevated levels of PAP may indicate testicular cancer, leukemia, and non-Hodgkin's lymphoma, as well as some noncancerous conditions.

As used herein, ovarian cancer is the most common cause of elevated CA 125, but cancers of the uterus, cervix, pancreas, liver, colon, breast, lung, and digestive tract can also raise CA 125 levels. Several noncancerous conditions can also elevate CA 125. CA 125 is mainly used to monitor the treatment of ovarian cancer.

As used herein “Carcinoembryonic antigen (CEA)” is normally found in small amounts in the blood. Colorectal cancer is the most common cancer that raises this tumor marker. Several other cancers can also raise levels of carcinoembryonic antigen.

As used herein “Alpha-fetoprotein (AFP)” is normally elevated in pregnant women since it is produced by the fetus. However, AFP is not usually found in the blood of adults. In men, and in women who are not pregnant, an elevated level of AFP may indicate liver cancer or cancer of the ovary or testicle. Noncancerous conditions may also cause elevated AFP levels.

As used herein “Human chorionic gondadotropin (HCG)” is another substance that appears normally in pregnancy and is produced by the placenta. If pregnancy is ruled out, HCG may indicate cancer in the testis, ovary, liver, stomach, pancreas, and lung. Marijuana use can also raise HCG levels.

As used herein “CA 19-9” marker is associated with cancers in the colon, stomach, and bile duct. Elevated levels of CA 19-9 may indicate advanced cancer in the pancreas, but it is also associated with noncancerous conditions, including gallstones, pancreatitis, cirrhosis of the liver, and cholecystitis.

As used herein “CA 15-3” marker is most useful in evaluating the effect of treatment for women with advanced breast cancer. Elevated levels of CA 15-3 are also associated with cancers of the ovary, lung, and prostate, as well as noncancerous conditions such as benign breast or ovarian disease, endometriosis, pelvic inflammatory disease, and hepatitis. Pregnancy and lactation also can raise CA 15-3 levels.

As used herein “CA 27-29” marker, like CA 15-3, is used to follow the course of treatment in women with advanced breast cancer. Cancers of the colon, stomach, kidney, lung, ovary, pancreas, uterus, and liver may also raise CA 27-29 levels. Noncancerous conditions associated with this substance are first trimester pregnancy, endometriosis, ovarian cysts, benign breast disease, kidney disease, and liver disease.

As used herein “Lactate dyhydrogenase (LDH)” is a protein that normally appears throughout the body in small amounts. Many cancers can raise LDH levels, so it is not useful in identifying a specific kind of cancer. Measuring LDH levels can be helpful in monitoring treatment for cancer. Noncancerous conditions that can raise LDH levels include heart failure, hypothyroidism, anemia, and lung or liver disease.

As used herein “Neuroson-specific enolase (NSE)” is associated with several cancers, but it is used most often to monitor treatment in patients with neuroblastoma or small cell lung cancer.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₄-C₁₂ alkenyl and so on.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl, C₃-C₁₂ alkynyl, C₄-C₁₂ alkynyl and so on.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As herein, “cycloalkyl” shall mean cyclic rings ofalkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or pluraly. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Fumis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds ofthe present invention may also form salts with basic amino acids such a lysine, arginine, etc. and with basic sugars such as N-methylglucamine, 2-amino-2-deoxyglucose, etc. and any other physiologically non-toxic basic substance.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method ofthe present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, asuitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modem Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

The present involves host-guest chemistry, CB7-Adma driven pre-targeting platform. Three copper-64-labeled Adma guest molecules (1-3) were synthesized and characterized. The in vivo profile of the ligands in pre-targeting strategy were evaluated with using a CB7-modified carcinoembryonic antigen (CEA) targeting humanized full-length antibody (CB7-M5A) as the secondary pre-targeting agent. The pre-targeting studies were performed in CEA+ and CEA− human pancreatic cancer mouse xenografts. The biodistribution of the pre-targeted Adma-radioligand was compared to that of a zirconium-89-labeled directly radiolabeled antibody. The dosimetry of the two antibody-based imaging approaches were compared. It was hypothesized that the highly stability, mutual high affinity and human compatibility of the proposed CB7-Adma pre-targeting agents provide a great basis for a pre-targeting platform.

### Materials and Methods

Three copper-64-labeled adamantane guest radioligands were developed and their in vitro stability, lipophilicity and in vivo blood half-life values were compared. The adamantane radioligands were analyzed for pretargeting using a cucurbit[7]uril modified carcinoembryonic antigen targeting full-length antibody hT84.66-M5A as the macromolecule pretargeting agent with two different dosing schedules. These molecules were evaluated for pretargeting in human pancreatic cancer BxPC3 and MIAPaCa-2 mouse xenografts using positron emission tomography and in vivo biodistribution. The dosimetry of the cucurbit[7]uril-adamantane pretargeting approach in human male was calculated and compared to that of the directly zirconium-89-labeled hT84.66-M5A.

### Reagents

All reagents were purchased from Sigma-Aldrich unless stated otherwise. p-SCN-Bn-NOTA×3 HCl and p-SCN-Bn-Deferoxamine were purchased from Macrocyclics. t-BOC—N-Amido-PEG3-Amine and t-Boc-N-amido-PEG7-amine were acquired from BroadPharm. The cucurbit[7]uril-azide (CB7-N₃) was synthesized by the Chemical Synthesis Core of Vanderbilt University, Tennessee USA. The M5A (hT84.66-M5A) antibody was received from Dr. Yazaki at City of Hope, California USA. The [⁸⁹Zr]Zr(C₂O₄)₂ and [⁶⁴Cu]CuCl₂ were purchased from Washington University School of Medicine MIR Cyclotron Facility. All cell culturing media solutions were purchased from VWR International unless mentioned otherwise. The reagents for the SDS PAGE and Western Blotting were purchased from ThermoFisher Scientific. PBS7.4 solution was prepared from phosphate buffered saline powder pH 7.4.

### Instruments

4-9 were purified with a reverse phase (RP) HPLC set up including an Agilent HPLC 1260 Infinity II LC System comprised of a 1260 Quat Pump VL, 1260 DAD WR and LabLogic Flow-RAM radio-HPLC Detector equipped with a LabLogic Systems Limited NaI Detector with a Luna 5 m C18(2) 100 Å LC column 250×10 mm (Phenomenex) column. The chemical purity of 5, 7, 9 and the radiochemical purity of 1-3 were measured with the same HPLC instrument using a Kinetex 5 m EVO C18 100 Å column, 150×4.6 mm (Phenomenex) analytical column. The radiochemical purity of [⁸⁹Zr]Zr-DFO-M5A was analyzed with BioScan AR-2000 radio-TLC scanner. The quality control of CB7-M5A and DFO-M5A was performed with BioRad NGC-Chormatography System including SystemPump10 and Multi UV/Vis-Conductivity detector using a Superdex 200 Increase 10/300GL Cytiva size exclusion column (SEC). The radioactivity of log D, blood half-life, cell internalization and in vivo biodistribution samples were measured with Hidex Automatic Gamma Counter. The radioactivity of the radiotracer doses was measured with a CRC-55tR Capintec Inc dose calibrator. 4-9 were characterized with a 500 MHz Bruker Avance III proton nuclear magnetic resonance spectroscopy and Q-Exactive HF (Thermo-Fisher) Orbi-trap mass spectrometer high-resolution mass spectrometry instrument.

### Liquid Chromatography Methods

The RP HPLC method used to purify 4-9 involved the use of 0.1% trifluoroacetic acid (TFA) in H₂O and 0.1% TFA in acetonitrile as solvents A and B, where % solvent B remained 5 for the first minute followed by increasing the solvent B % to 95 over a 24-minute gradient with a flow rate of 4 mL/min. The RP HPLC method used to determine the radiochemical or chemical purity of 1-3 and 5, 7 and 9 involved the use of the same solvents as in the previous method. The % solvent B remained at 5 the first minute of the run followed by gradient from 5 to 95 over 17 minutes using a 1 mL/min flow rate. The final RP HPLC method was used to analyze the in vitro plasma stability samples of 1-3. The solvent A and B were H₂O and acetonitrile. The % of solvent B remained at 0 for the first 5 minutes of the run. Next the solvent B % was increased to 95 over a 15-minute gradient.

The FPLC—size exclusion method used to analyze the purity of the CB7-M5A and DFO-M5A involved the use of PBS7.4 as the only solvent over a 70-minute period with a flow rate of 0.7 mL/min.

### Synthesis of NBOC-Adma (4)

NBOC-ethylenediamine (287 mg; 1.81 mmol; 1 eq.), adamantane-1-carbaldehyde (306 mg; 1.87 mmol; 1 eq.) and triethylamine (14.5 mg; 0.14 mmol; 0.1 eq.) were dissolved in methanol (50 mL). The reaction was stirred for 30 minutes in room temperature before the addition of sodium triacetoxyborohydride (596 mg; 2.81 mmol; 1.5 eq.). The reaction solution was stirred for 60 minutes in room temperature after which the solvent was evaporated off Water (50 mL) was added to the crude product material. The water phase was extracted with diethyl ether (3×40 mL) followed by combining and evaporating the diethyl ether phases to yield oily material. Iodomethane (30 mL) and sodium hydroxide (119 mg; 2.98 mmol; 1.6 eq.) were added to the reaction vial. The methylation reaction was stirred for 2 hours, which was followed by evaporating the iodomethane and adding fresh iodomethane which was repeated once more. The solvent was evaporated and replaced with dichloromethane. Undissolved sodium hydroxide was filtered out and the dichloromethane evaporated. The crude product NBOC-Adma material was dissolved in acetonitrile (7 mL). The crude product was purified with a UV-Vis-HPLC chromatography system using a C18 semipreparative column. The product fractions were combined and concentrated. Reaction yield: 60.0% (366 mg) Chemical purity: 95.8%

HRMS (ESI)(+) m/z calculated for C₂₀H₃₇N₂O₂⁺ [M]⁺: 337.2850 and measured: 337.2849.

¹H NMR (500 MHz, methanol-d4); 3.580-3524 (m, 2H), 3.518-3.472 (m, 2H), 3.237 (s, 6H), 3.182 (s, 2H), 2.053 (s, 3H), 1.863 (d, 6H), 1.781 (q, 6H), 1.454 (s, 9H).

### Synthesis of NOTA-ADMA (5)

4 (44.6 mg; 0.13 mmol) was dissolved in dichloromethane (3 mL) and trifluoroacetic acid (1 mL) and the reaction at room temperature for 20 minutes. The solvent and acid were evaporated and the deprotected molecule was redissolved in dimethyl sulfoxide (1 mL). p-SCN-Bn-NOTA×3 HCl (38.7 mg; 0.07 mmol; 0.5 eq.) and N,N-diisopropylethylamine (20.0 mg; 0.16 mmol; 1.2 eq.) was added to the reaction solution and stirred for 1 hour. The product was purified with a C18 semipreparative column with a UV-Vis-HPLC chromatography system detecting λ=254 nm. Reaction yield: 24.2% (11.7 mg). Chemical purity: 97.6%

HRMS (ESI)(+) m/z calculated for C₃₅H₅₅N₆O₆S⁺ [M]⁺: 687.3898 and measured: 687.3899.

¹H-NMR (500 MHz, methanol-d4); 7.343 (d, 2H), 7.292 (d, 2H), 4.142 (t, 2H), 3.992-3.704 (m, 4H), 3.673 (t, 2H), 3.477-3.322 (m, 4H), 3.279 (s, 6H), 3.228 (s, 4H), 3.192-3.139 (m, 2H), 3.138-3.003 (m, 2H), 2.999-2.902 (m, 1H), 2.900-2.745 (m, 2H), 2.744-2.540 (m, 2H), 2.046 (s, 3H), 1.868 (d, 6H), 1.771 (q, 6H).

### Synthesis of NBOC-PEG₃-ADMA (6)

Adamantane-1-carbaldehyde (213 mg; 1.3 mmol; 1 eq.), t-BOC—N-Amido-PEG3-Amide (356 mg; 1.2 mmol; 1.1 eq.) and triethylamine (21.2 mg; 0.2 mmol; 0.2 eq.) were dissolved in methanol (50 mL). Reaction solution was stirred for 30 minutes at room temperature followed by an addition of sodium triacetoxyborohydride (509 mg; 2.4 mmol; 1.8 eq.). The reaction was stirred overnight at room temperature. The solvent was evaporated off followed by an addition of diethyl ether (10 mL). The undissolved material was filtered, and the diethyl ether phase was collected and evaporated. The crude material was dissolved in iodomethane (20 mL) along with triethylamine (21.2 mg; 0.2 mmol; 0.2 eq.) and sodium hydroxide (110 mg; 2.8 mmol; 2 eq.). The methylation reaction was stirred at room temperature for 2 hours. The iodomethane was evaporated and replaced with fresh iodomethane twice (20 mL). Finally iodomethane was evaporated off and replaced with dichloromethane. Undissolved sodium hydroxide was filtered out and the dichloromethane evaporated. The crude product NBOC-PEG₃-Adma material was dissolved in acetonitrile (7 mL) and methanol (3 mL). The dissolved material was injected to UV-Vis-HPLC chromatography system for purification with a C18 semipreparative column. The product fractions were combined and concentrated. Reaction yield 68.0% (388 mg). Chemical purity: 99.8%

HRMS (ESI)(+) m/z calculated for C₂₆H₄₉N₂O₅⁺ [M]⁺: 469.3636 and measured: 469.3639.

¹H NMR (500 MHz, methanol-d4); 3.977-3.928 (m, 2H), 3.718-3.636 (m, 8H), 3.629-3.595 (m, 2H), 3.497 (t, 2H), 3.274-3.237 (m, 6H), 3.236-3.181 (m, 4H), 2.048 (m, 3H), 1.861 (d, 6H), 1.776 (q, 6H), 1.440 (s, 9H).

### Synthesis of NOTA-PEG₃-ADMA (7)

6 (52 mg; 0.11 mmol; 1 eq.) was dissolved to dichloromethane (3 mL) and trifluoroacetic acid (2 mL) and the reaction was stirred at room temperature for 20 minutes. The solvent and acid were evaporated with a rotary evaporator. The material was redissolved in dimethyl sulfoxide (1 mL). 2-S-(4-Isothiocyanatobenzyl)-1,4,7-triazacyclononane-1,4,7-triacetic acid (p-SCN-Bn-NOTA; 17.4 mg; 0.03 mmol; 0.3 eq.) was added to the reaction vial along with N,N-diisopropylethylamine (18.6 mg; 0.14 mmol; 1.3 eq.). The reaction was stirred for 3 hours. The product was purified with a C18 semipreparative column with a UV-Vis-HPLC chromatograph. The product fractions were combined and concentrated. Reaction yield 53.2% (16.1 mg). Chemical purity: 97.2%

HRMS (ESI)(+) m/z calculated for C₄₁H₆₇N₆O₉S⁺[M]⁺: 819.4685 and measured: 819.4755. ¹H-NMR (500 MHz, methanol-d4); 7.378 (d, 2H), 7.276 (d, 2H), 4.017-3.916 (m, 4H), 3.916-3.800 (m, 2H), 3.699 (s, 2H), 3.728-3.671 (m, 2H), 3.670-3.650 (m, 8H), 3.649-3.612 (m, 2H), 3.434-3.325 (m, 4H), 3.236 (s, 6H), 3.209 (s, 4H), 3.184-3.133 (m, 2H), 3.131-2.951 (m, 2H), 2.948-2.885 (m, 1H), 2.882-2.740 (m, 2H), 2.738-2.572 (m, 2H), 2.033 (s, 3H), 1.842 (d, 6H), 1.762 (q, 6H).

### Synthesis of NBOC-PEG₇-ADMA (8)

Adamantane-1-carbaldehyde (188 mg; 1.1 mmol), t-Boc-N-amido-PEG7-amine (215 mg; 0.46 mmol) and triethylamine (21.8 mg; 0.22 mmol) were dissolved in methanol (30 mL). The solution was stirred at room temperature for 1 hour. Sodium triacetoxyborohydride (543 mg; 2.56 mmol) was added to the reaction solution and the stirring was continued for 2 hours. The solvent was evaporated off and the crude material was mixed with diethyl ether (10 mL). Undissolved sodium triacetoxyborohydride was filtered off and washed twice with diethyl ether (2×10 mL). Diethyl ether phases were combined and concentrated with a rotary evaporator. The crude material (366 mg) was dissolved in iodomethane (10 mL). NaOH (110 mg; 2.75 mmol) was added to the reaction solution. The methylation reaction mixture was stirred at RT for 2 hours which was followed by evaporation of the iodomethane. Additional fresh iodomethane (10 mL) was added the reaction vial. The iodomethane change was repeated twice more. Iodomethane was evaporated off one last time. The crude product was dissolved in ACN (4 mL) and purified with a RP-HPLC C18 semipreparative column. Due to the lack of UV absorption of the product molecule, all eluted material was collected in fractions. The fraction containing the product was determined with MS. The product fractions were combined and the solvent was evaporated off Reaction yield: 31.3% (93.1 mg) Chemical purity: 98.9%

HRMS (ESI)(+) m/z calculated for C₃₄H₆₅N₂O₉⁺ [M]⁺: 645.4685 and measured: 645.4679.

¹H-NMR (500 MHz, methanol-d4); 3.972-3.933 (s, m H), 3.670-3.672 (m, 2H), 3.671-3.622 (m, 24H), 3.621-3.598 (m, 2H), 3.506 (t, 2H), 3.246 (s, 6H), 3.233-3.206 (m, 4H), 2.046 (s, 3H), 1.858 (d, 6H), 1.770 (q, 6H), 1.438 (s, 9H).

### Synthesis of NOTA-PEG₇-ADMA (9)

8 (21.4 mg; 0.033 mmol) was dissolved in dichloromethane (3 mL) and trifluoro acetic acid (0.6 mL). The solution was stirred at RT for 15 minutes. Dichloromethane and trifluoroacetic acid were evaporated off. The dried material and p-SCN-Bn-NOTA (15.7 mg; 0.028 mmol) was dissolved in dimethyl sulfoxide (1 mL) mixed with N,N-diisopropylethylamine (18.55 mg; 0.14 mmol). The reaction was stirred for 40 minutes at RT. Dimethyl sulfoxide was evaporated off under air flow over night at RT. The crude product was dissolved in acetonitrile (2 mL). The product was purified with a RP-HPLC C18 semipreparative column. Reaction yield: 45.0% (12.6 mg) Chemical purity: 95.8%

HRMS (ESI)(+) m/z calculated for C₄₉H₈₃N₆O₁₃S⁺[M]⁺: 995.5733 and measured: 995.5724.

¹H-NMR (500 MHz, methanol-d4); 7.377 (d, 2H), 7.270 (d, 2H), 4.024-3.911 (m, 4H), 3.910-3.795 (m, 2H), 3.770 (s, 2H), 3.694-3.671 (m, 2H), 3.664-3.614 (m, 24H), 3.612-3.544 (m, 2H), 3.429-3.320 (m, 4H), 3.237 (s, 6H), 3.214 (s, 4H), 3.186-3.143 (m, 2H), 3.141-2.985 (m, 2H), 2.974-2.887 (m, 1H), 2.886-2.737 (m, 2H), 2.755-2.544 (m, 2H), 2.041 (s, 3H), 1.852 (d, 6H), 1.771 (q, 6H).

### In Vitro Stability of the Adamantane Radioligands

TABLE 1
In vitro stability of [⁶⁴Cu]Cu-NOTA-Adma (1),
[⁶⁴Cu]Cu-NOTA-PEG₃-Adma (2) and [⁶⁴Cu]Cu-NOTA-PEG₇-
Adma (3) in PBS7.4 at 37 °C.

| | 1 | 2 | 3 |
|---|---|---|---|
| 1 h | 98.2 ± 1.9 | 96.8 ± 2.0 | 93.4 ± 7.2 |
| 6 h | 99.3 ± 1.0 | 97.4 ± 3.1 | 91.0 ± 0.5 |
| 24 h | 94.9 ± 0.2 | 99.0 ± 0.8 | 94.2 ± 3.2 |

TABLE 2
In vitro stability of [⁶⁴Cu]Cu-NOTA-Adma (1), [⁶⁴Cu]Cu-NOTA-
PEG₃-Adma (2) and [⁶⁴Cu]Cu-NOTA-PEG₇-Adma (3) in bovine
plasma at 37 °C.

| | 1 | 2 | 3 |
|---|---|---|---|
| 1 h | 99.6 ± 0.6 | 95.1 ± 0.2 | 98.6 ± 0.1 |
| 6 h | 98.1 ± 0.4 | 93.9 ± 0.6 | 96.8 ± 0.9 |
| 24 h | 95.2 ± 0.9 | 90.6 ± 1.6 | 97.5 ± 0.6 |

### Cell Culturing

All cell lines were purchased from ATCC (Manassas, VA). BxPC3 human pancreatic adenocarcinoma cells were maintained in RPMI-1640 medium with 0.3 g/l glutamine, 25 mM HEPES, 1% (vol/vol) Penicillin-Streptomycin and 10% (vol/vol) fetal bovine serum. MIAPaCa-2 human pancreatic carcinoma cells were grown in DMEM/High glucose medium containing 4 mM L-glutamine, 4.5 g/L glucose, and sodium pyruvate, 1% (vol/vol) Penicillin-Streptomycin, 10% (vol/vol) fetal bovine serum and 2.5% (vol/vol) Horse Serum (donor herd) (Sigma-Aldrich). Both cells were kept in a 37 °C environment containing 5% CO₂ and extracted using 0.25% Trypsin-EDTA.

### Animals

All animals were female nude mice (NU/NU Charles River). The mice were housed in static microisolator caging with corn cob bedding (The Anderson Bed-o'Cobs ⅛″). The food provided was Lab Diet 5053 Irradiated PicoLab Rodent Diet 20. The mice were housed in 12-hour light/dark cycles. The mice were maintained in maximum isolation rooms, which are health monitored quarterly for pathogens and ecto/endo parasites quarterly. For experiments including xenografts, the mice were injected subcutaneously with BxPC3 or MIAPaCa-2 cells (5-6 □10⁶ cells in respective media and matrigel, 1:1% V/V; 150 L). The mice were used once the tumor volume reached ~100 mm³ which occurred 5-6 weeks after implantation of the xenografts. All experiments involving laboratory animals were performed in accordance with Institutional Animal Care and Use Committee at Stony Brook Medicine.

### Blood Half-Life Studies of the Adma Radioligands

In total 3-5 μL of blood was collected from each mouse per one timepoint. The blood was collected using capillary tubes (VWR Micro-Hematocrit tubes with HEPARIN).

For the analysis, a two-phase decay curve was formed based on the plotted % ID/g (blood) values as a function of time. The y₀ of the curve was set to 50% ID/g, because it was estimated that 100% of the activity is in the blood pool at 0 min (x₀=0) and that the total mass of the blood pool of a nude female mouse is 2 g. Based on the two-phase decay curve, each radioligand's percental fast (t_{1/2fast}) and slow phase (t_{1/2slow}) values were used to calculate the radioligand's weighted blood half-life with equation one.

$${t}_{1 / 2} = \frac{( {t}_{1 / 2 slow} \times {\%}_{fast} ) + ( {t}_{1 / 2 fast} \times {\%}_{slow} )}{100} \qquad ( 1 )$$

### Renal Excretion of 2 in Healthy Nude Mice

Healthy female nude mice (n=3) were injected intravenously via the tail vein with 2 (7.1±0.3 MBq/nmol; 10.7±0.4 MBq in 150 μL of PBS). 20 and 60 min post injection the urine was collected to preweighed Eppendorf tubes. The tubes were weighed and counted on a gamma counter to determine the % ID/g value of each sample.

TABLE 3
The % ID/g of collected urine samples
in healthy nude female mice.

| | 20 min post | 60 min post |
|---|---|---|
| | injection of 2 | injection of 2 |
| % ID/g (urine) | 684 ± 206 | −1047 ± 454 |

### Western Blotting for Carcinoembryonic Antigen

The cell lysates for BxPC3 and MIAPaCa-2 were obtained by incubating the cells with the Cell Lysis Buffer II containing PMSF protease inhibitor and Halt™ Protease inhibitor cocktail. Protein quantification was performed with Pierce™ BCA Protein Assay Kit. The protein lysates were prepared in NuPAGE LDS Sample buffer. Each protein sample (20 g) was separated by SDS-PAGE using a NuPAGE™ 4 to 12%, Bis-Tris, 1.0-1.5 mm, Mini Protein Gels at 100V for 15 minutes followed by an increase to 150V for an additional 60 minutes. Proteins were transferred electrophoretically onto Invitrolon™ PVDF/Filter Paper Sandwiches at 100 V for 60 minutes. Membranes were blocked in 5% Non-Fat Dry Milk prepared in TBS Tween™20 Buffer for 1 hour and incubated with the primary antibodies overnight at 4 °C. The primary antibodies used were CEA Monoclonal Antibody (H.426.3) (ThermoFisher Scientific), beta Tubulin Loading Control Monoclonal Antibody (BT7R) (ThermoFisher Scientific) and the hT84.66-M5A. The membranes were washed with TBS Tween™20 Buffer followed by incubation with the respective secondary antibodies (Goat anti-Mouse IgG (H+L), Superclonal Recombinant Secondary Antibody, HRP and Goat anti-human IgG FC Antibody, Horseradish Peroxidase HRP conjugate, cross absorbed) at room temperature for 1 hour. Western blots were developed using Pierce™ Fast Western Blot Kit, ECL Substrate. Immunoreactivity was detected on a Konica Minolta SRX-101A Medical Film Processor.

### In Vivo Biodistribution Procedure

The mice were euthanized by inhalation of 5% isoflurane with a 2.0 mL/min flow followed by a cervical dislocation. Blood, tumor, heart, lungs, stomach, liver, pancreas, spleen, small intestine, large intestine, kidneys, muscle and bone were harvested and placed in pre-weighed tubes. The contents of the small and large intestines were collected along with the tissue. To determine each organ sample's % ID/g value, the samples were weighed and counted with a gamma counter.

### In Vivo Pretargeted 1, 2 and 3

TABLE 4
In vivo biodistribution organ % ID/g values of BxPC3
tumor bearing female nude mice administered with 1
72 h post CB7-M5A injection. The timepoints represent
the amount of time passed post injection of 1.

| | CB7-M5A & 1 | | | 1 |
|---|---|---|---|---|
| | 4 h | 8 h | 24 h | 24 h (CTRL) |
| blood | 1.8 ± 0.1 | 2.4 ± 0.2 | 2.1 ± 0.5 | 0.1 ± 0.0 |
| tumor | 5.3 ± 0.8 | 4.6 ± 0.9 | 8.9 ± 2.0 | 0.0 ± 0.0 |
| heart | 0.5 ± 0.1 | 0.8 ± 0.2 | 0.5 ± 0.1 | 0.0 ± 0.1 |
| lungs | 0.8 ± 0.2 | 0.8 ± 0.5 | 0.6 ± 0.2 | 0.1 ± 0.1 |
| stomach | 0.4 ± 0.0 | 0.5 ± 0.2 | 0.5 ± 0.2 | 0.1 ± 0.1 |
| liver | 0.2 ± 0.0 | 0.3 ± 0.2 | 0.2 ± 0.2 | 0.1 ± 0.2 |
| pancreas | 0.3 ± 0.1 | 0.3 ± 0.1 | 0.2 ± 0.1 | 0.0 ± 0.0 |
| spleen | 0.7 ± 0.1 | 0.6 ± 0.3 | 0.7 ± 0.1 | 0.2 ± 0.2 |
| s. intestine | 0.4 ± 0.1 | 0.5 ± 0.6 | 0.1 ± 0.0 | 0.1 ± 0.1 |
| l. intestine | 7.5 ± 1.0 | 1.8 ± 0.7 | 0.1 ± 0.0 | 0.2 ± 0.2 |
| kidneys | 0.9 ± 0.1 | 0.8 ± 0.4 | 0.7 ± 0.1 | 0.2 ± 0.2 |
| muscle | 0.2 ± 0.1 | 0.3 ± 0.0 | 0.2 ± 0.1 | 0.0 ± 0.0 |
| bone | 0.2 ± 0.0 | 0.3 ± 0.2 | 0.2 ± 0.1 | 0.0 ± 0.0 |

TABLE 5
In vivo biodistribution organ % ID/g values of BxPC3
tumor bearing female nude mice administered with 2
72 h post CB7-M5A injection. The timepoints represent
the amount of time passed post injection of 2.

| | | | | | 2 |
|---|---|---|---|---|---|
| | CB7-M5A & 2 | | | | 24 h |
| | 2 h | 4 h | 8 h | 24 h | (CTRL) |
| blood | 2.5 ± 0.6 | 2.4 ± 1.1 | 1.9 ± 0.5 | 2.1 ± 0.1 | 0.0 ± 0.0 |
| tumor | 3.3 ± 0.8 | 3.5 ± 1.1 | 5.9 ± 1.6 | 12.0 ± 0.9 | 0.0 ± 0.0 |
| heart | 0.7 ± 0.1 | 0.7 ± 0.3 | 0.6 ± 0.2 | 0.6 ± 0.1 | 0.0 ± 0.0 |
| lungs | 1.2 ± 0.4 | 0.7 ± 0.3 | 0.7 ± 0.1 | 1.0 ± 0.3 | 0.0 ± 0.0 |
| stomach | 0.6 ± 0.2 | 0.4 ± 0.2 | 0.5 ± 0.1 | 0.5 ± 0.2 | 0.0 ± 0.0 |
| liver | 0.3 ± 0.1 | 0.4 ± 0.4 | 0.1 ± 0.0 | 0.1 ± 0.0 | 0.0 ± 0.0 |
| pancreas | 0.3 ± 0.1 | 0.2 ± 0.1 | 0.2 ± 0.1 | 0.1 ± 0.0 | 0.0 ± 0.0 |
| spleen | 0.9 ± 0.3 | 0.8 ± 0.2 | 0.6 ± 0.2 | 0.6 ± 0.1 | 0.0 ± 0.0 |
| s. intestine | 0.9 ± 0.3 | 0.3 ± 0.1 | 0.2 ± 0.0 | 0.1 ± 0.0 | 0.0 ± 0.0 |
| l. intestine | 7.2 ± 2.3 | 7.0 ± 1.2 | 0.8 ± 0.3 | 0.1 ± 0.0 | 0.0 ± 0.0 |
| kidneys | 1.0 ± 0.1 | 0.8 ± 0.2 | 0.7 ± 0.2 | 0.8 ± 0.2 | 0.1 ± 0.0 |
| muscle | 0.3 ± 0.1 | 0.2 ± 0.1 | 0.2 ± 0.1 | 0.2 ± 0.0 | 0.0 ± 0.0 |
| bone | 0.3 ± 0.1 | 0.2 ± 0.1 | 0.2 ± 0.1 | 0.2 ± 0.0 | 0.0 ± 0.0 |

TABLE 6
In vivo biodistribution organ % ID/g values of BxPC3
tumor bearing female nude mice administered with 3
72 h post CB7-M5A injection. The timepoints represent
the amount of time passed post injection of 3.

| | CB7-M5A & 3 | | | 3 |
|---|---|---|---|---|
| | 4 h | 8 h | 24 h | 24 h (CTRL) |
| blood | 1.1 ± 0.3 | 0.7 ± 0.2 | 0.9 ± 0.3 | 0.0 ± 0.0 |
| tumor | 2.2 ± 0.8 | 1.7 ± 0.9 | 3.9 ± 2.1 | 0.0 ± 0.0 |
| heart | 0.3 ± 0.1 | 0.2 ± 0.1 | 0.2 ± 0.1 | 0.0 ± 0.0 |
| lungs | 0.4 ± 0.0 | 0.4 ± 0.1 | 0.3 ± 0.1 | 0.0 ± 0.0 |
| stomach | 0.2 ± 0.1 | 0.2 ± 0.0 | 0.2 ± 0.1 | 0.0 ± 0.0 |
| liver | 0.1 ± 0.1 | 0.0 ± 0.0 | 0.0 ± 0.0 | 0.0 ± 0.0 |
| pancreas | 0.1 ± 0.0 | 0.1 ± 0.0 | 0.1 ± 0.0 | 0.0 ± 0.0 |
| spleen | 0.5 ± 0.1 | 0.4 ± 0.1 | 0.4 ± 0.1 | 0.1 ± 0.0 |
| s. intestine | 0.4 ± 0.3 | 0.1 ± 0.0 | 0.1 ± 0.0 | 0.0 ± 0.0 |
| l. intestine | 14.6 ± 6.0 | 2.1 ± 0.7 | 0.2 ± 0.1 | 0.2 ± 0.1 |
| kidneys | 0.7 ± 0.1 | 0.6 ± 0.1 | 0.6 ± 0.1 | 0.1 ± 0.0 |
| muscle | 0.1 ± 0.1 | 0.1 ± 0.0 | 0.1 ± 0.0 | 0.0 ± 0.0 |
| bone | 0.1 ± 0.0 | 0.1 ± 0.0 | 0.1 ± 0.0 | 0.0 ± 0.0 |

TABLE 7
In vivo biodistribution organ % ID/g values of BxPC3
tumor bearing female nude mice administered with
2 144 h post CB7-M5A injection. The timepoints represent
the amount of time passed post injection of 2.

| | CB7-M5A & 2 | | |
|---|---|---|---|
| | 4 h | 8 h | 24 h |
| blood | 0.6 ± 0.3 | 0.3 ± 0.2 | 0.3 ± 0.0 |
| tumor | 3.2 ± 1.4 | 3.0 ± 1.1 | 5.3 ± 1.4 |
| heart | 0.1 ± 0.0 | 0.2 ± 0.1 | 0.2 ± 0.1 |
| lungs | 0.3 ± 0.1 | 0.2 ± 0.1 | 0.4 ± 0.1 |
| stomach | 0.2 ± 0.0 | 0.2 ± 0.1 | 0.2 ± 0.1 |
| liver | 0.1 ± 0.0 | 0.1 ± 0.0 | 0.2 ± 0.1 |
| pancreas | 0.1 ± 0.0 | 0.1 ± 0.0 | 0.1 ± 0.0 |
| spleen | 1.4 ± 0.7 | 1.0 ± 0.2 | 0.9 ± 0.1 |
| s. intestine | 0.8 ± 0.6 | 0.2 ± 0.1 | 0.2 ± 0.1 |
| l. intestine | 8.0 ± 0.4 | 4.4 ± 1.5 | 0.3 ± 0.1 |
| kidneys | 0.9 ± 0.2 | 0.6 ± 0.2 | 0.6 ± 0.1 |
| muscle | 0.2 ± 0.2 | 0.1 ± 0.1 | 0.1 ± 0.0 |
| bone | 0.3 ± 0.2 | 0.1 ± 0.0 | 0.1 ± 0.0 |

TABLE 8
Tumor-to-organ ratios for pretargeted 1, 2 and 3 in BxPC3 tumor
bearing female nude mice performed with a 72 h or 144 h lag time.

| | | | tumor | tumor to | tumor |
|---|---|---|---|---|---|
| | | | to blood | l. intestine | to muscle |
| pretargeted 1 | 4 | h | 3.0 ± 0.6 | 0.7 ± 0.1 | 24.2 ± 7.4 |
| (72 h lag time) | 8 | h | 1.9 ± 0.3 | 3.0 ± 1.4 | 18.3 ± 2.7 |
| | 24 | h | 4.7 ± 1.8 | 59.2 ± 30.7 | 46.6 ± 30.0 |
| pretargeted 2 | 4 | h | 2.3 ± 1.5 | 0.5 ± 0.1 | 21.9 ± 9.9 |
| (72 h lag time) | 8 | h | 3.2 ± 0.6 | 8.4 ± 3.8 | 39.8 ± 9.8 |
| | 24 | h | 5.8 ± 0.4 | 75.3 ± 29.5 | 84.2 ± 19.0 |
| pretargeted 3 | 4 | h | 2.2 ± 1.1 | 0.2 ± 0.0 | 17.1 ± 4.3 |
| (72 h lag time) | 8 | h | 2.6 ± 1.5 | 1.0 ± 0.8 | 30.6 ± 16.4 |
| | 24 | h | 4.5 ± 1.9 | 25.0 ± 18.0 | 59.8 ± 39.4 |
| pretargeted 2 | 4 | h | 5.7 ± 1.6 | 0.4 ± 0.2 | 43.4 ± 15.1 |
| (144 h lag time) | 8 | h | 12.2 ± 7.6 | 0.6 ± 0.2 | 39.7 ± 20.7 |
| | 24 | h | 16.7 ± 4.6 | 23.9 ± 6.6 | 67.2 ± 28.3 |

### Dosimetry

TABLE 9
The estimated organ dose values of an adult human male
from a dose of pretargeted [⁶⁴Cu]Cu-NOTA-PEG₃-Adma (2).

| | [⁸⁹Zr]Zr-DFO-M5A | pretargeted | fold |
|---|---|---|---|
| target organ | (μSv/MBq)¹ | 2 (μSv/MBq) | change |
| adrenals | 280 | 2.5 | 110 |
| brain | 3.5 | 0.016 | 210 |
| esophagus | 150 | 1.0 | 150 |
| eyes | 3.9 | 0.022 | 180 |
| gallbladder wall | 280 | 2.7 | 100 |
| left colon | 580 | 94 | 6.2 |
| small intestine | 230 | 10 | 22 |
| stomach wall | 220 | 2.7 | 81 |
| right colon | 410 | 50 | 8.2 |
| rectum | 57 | 1.0 | 57 |
| heart wall | 640 | 12 | 52 |
| kidneys | 520 | 14 | 38 |
| liver | 530 | 7.2 | 74 |
| lungs | 140 | 1.8 | 79 |
| pancreas | 280 | 5.1 | 55 |
| prostate | 46 | 3.3 | 14 |
| salivary glands | 6.4 | 0.037 | 170 |
| red marrow | 58 | 0.83 | 69 |
| osteogenic cells | 37 | 0.49 | 76 |
| spleen | 310 | 5.1 | 62 |
| tumor | 18000 | 670 | 26 |
| thymus | 160 | 0.97 | 160 |
| thyroid | 32 | 0.20 | 160 |
| urinary bladder wall | 73 | 2.2 | 33 |
| total body | 62 | 2.0 | 30 |
| effective dose | 860 | 40 | 22 |

### Pet Imaging Procedure

Prior to each scan, the mice were anesthetized by inhalation of 2.5% isoflurane with a 2.0 mL/min flow for 5 minutes. The mice were kept under anesthesia and kept warm with an infrared lamp for the duration of the scan.

The 4 and 8 h timepoint imaging studies were performed with 10-minute scans while the 24 h timepoint images were acquired with 30-minute scans. The energy and time coincidence windows used for the scans were 350-650 keV and 3.432 ns. The imaging was performed on a small animal Siemens Inveon PET/CT. Data from all possible lines of response (LOR) were saved in the list mode raw data format. The raw data was then binned into 3D sinograms with a span of 3 and ring difference of 79. The images were reconstructed into transaxial slices (128×128×159) with voxel sizes of 0.800000×0.800000×0.7999150 mm³, using the MAP algorithm with 16 subsets and 18 iterations at a beta value of 0.00427838.

### EXAMPLES

### Development of the Radiolabeling Precursors

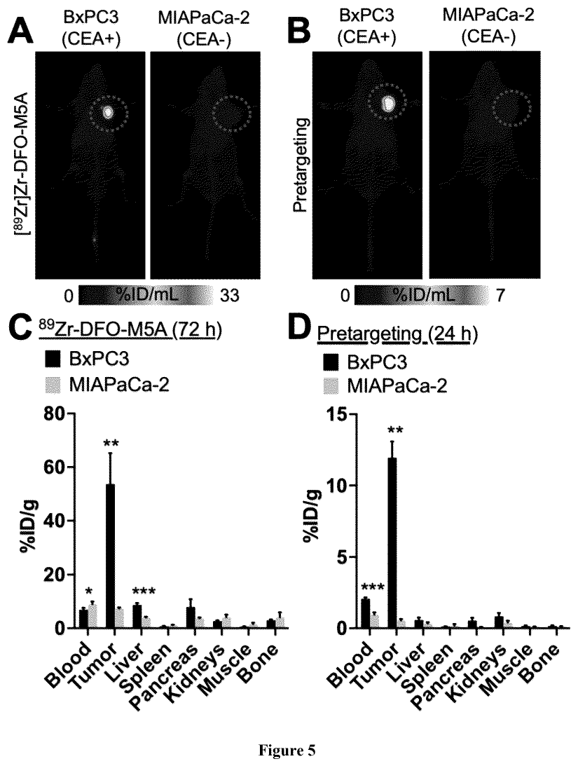

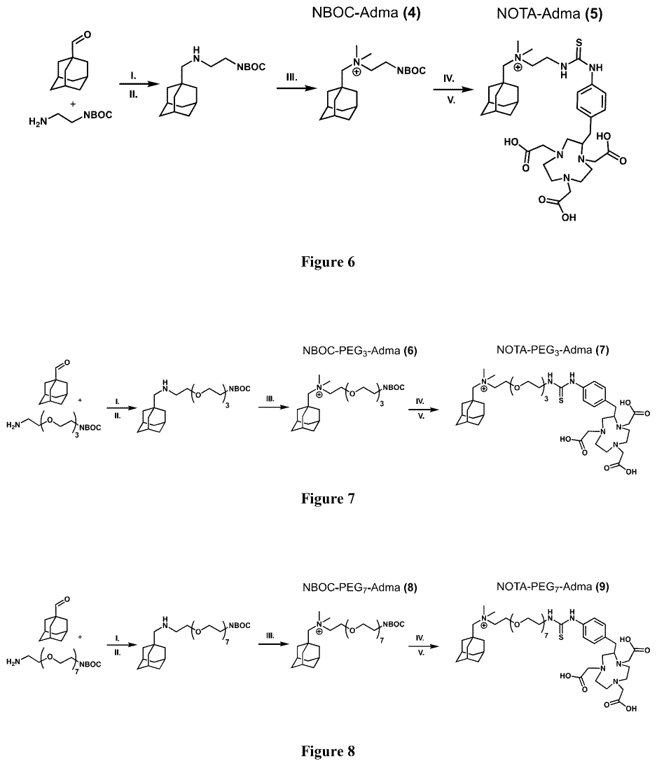

Detailed synthesis and characterization of the precursors for the Adma radioligands, 1-3 are described in FIGS. 6-8. To synthesize 1-3, [⁶⁴Cu]CuCl₂ in 0.05 M HCl (1.5-7.4 μL; 47-260 MBq) was mixed with 0.2 M NH₄OAc (pH 5.5; 50-150 L) in an Eppendorf tube. Respective precursor for 1/2/3 (1-L in DMSO) was added to the solution followed by incubating the reaction at room temperature for 10 minutes (“min”). The reaction was monitored with radio-HPLC with a method described in the present invention. Due to the high radiolabeling yield, no purification was required.

### Synthesis and Characterization of Modified M5A Molecules

CB7-M5A and deferoxamine conjugated M5A (DFO-M5A) were prepared as previously described (Jallinoja, 2021 and Jallinoja, 2022). The quality control of both modified antibodies was performed with size exclusion chromatography and the immunoreactivity was determined via cellular binding, Lindmo assay as previously reported (Jallinoja, 2022 and Lindmo 1986). The number of CB7 moieties per monoclonal antibody (mAb) was determined as previously reported (Jallinoja, 2021).

### ⁸⁹Zr-labeling of DFO-M5A

[⁸⁹Zr]Zr(C₂O₄)₂ in 0.1 M oxalic acid (86.2 MBq; 46 L) was neutralized to pH 7.4 using 1 M NaHCO₃. DFO-M5A (900 g; 6 nmol in 450 L in phosphate buffer saline pH 7.4 (PBS)) was added to the solution of zirconium-89 and the labeling reaction was incubated in room temperature for 1 h. The labeling reaction yield was checked with radio thin layer chromatography (radio-TLC) scanner using iTLC-SG-Glass microfiber chromatography paper and 50 mM ethylenediaminetetraacetic acid as the mobile phase. The synthesized [⁸⁹Zr]Zr-DFO-M5A was purified with a PD10 desalting column using PBS as the elution buffer. The radiochemical purity of the purified product was determined using the previously described radio-TLC method.

### In Vitro Stability and Plasma Protein Binding of 1-3

In vitro stability of 1-3 was studied in PBS and in bovine plasma at 37 °C. First, freshly synthesized radioligand (1/2/3; 1.5 nmol; 3.0-3.9 MBq in 100 L of 0.2 M NH₄OAc pH 5.5) was added to an Eppendorf with PBS (1 mL). The samples were incubated at 37 °C for 1, 6 or 24 h after which they were analyzed with radio high performance liquid chromatography (radio-HPLC). To study the ligands' stability in plasma, freshly synthesized 1/2/3 (1.4 MBq in 5 L of 0.2 M NH₄OAc pH 5.5) was mixed with bovine plasma (100 μL). The samples were incubated at 37 °C for 1, 6 or 24 h followed by an addition of cold acetonitrile (100 μL). The sample was centrifuged for 5 minutes (10000 rpm). Supernatant was collected and centrifuged for the second time after which the supernatant was diluted with H₂O (300 μL) and ran on a radio-HPLC. The formed pellet was measured for radioactivity and compared to the initial total activity to determine the protein bound fraction. Both stability assays were done in triplicate.

### Distribution Coefficient Measurement of the Guest Radioligands

Freshly synthesized 1/2/3 (586-610 kBq; 13 μL in 0.2 M NH₄OAc pH 5.5) was added to an Eppendorftube containing PBS (600 μL) and 1-octanol (600 μL). The mixture was stirred on a thermomixer for 10 minutes (900 rpm) at room temperature followed by centrifuging the sample for 5 minutes (1000 rcf). 200 μL of each phase was transferred, and the samples were weighed and counted on a gamma counter to determine the relative amount of radioactivity in each phase. The distribution coefficient (log D) value was calculated as log D=log₁₀(% radioligand in 1-octanol/% radioligand in PBS). The experiment was done in triplicate.

BxPC3 Cell Interaction with 2

Freshly synthesized 2 (354 kBq; 10 μL) or [⁶⁴Cu]CuCl₂ in 0.2 M NH₄OAc pH 5.5 (407 kBq; 10 L) was mixed with RPMI-1640 medium containing 0.3 g/l glutamine, 25 mM HEPES, 1% (vol/vol) Penicillin-Streptomycin and 10% (vol/vol) fetal bovine serum (12 mL). Media (1 mL) was measured to the wells of a six well plate which had been plated with 0.75×10⁶ BxPC3 cell the day prior. The cells were incubated with the media for 2, 4 or 6 h at 37 °C followed by treating the cells with 0.05 M glycine (pH 2.8). Finally, the cells were lysed by incubating them with 1.0 M NaOH. The media, glycine and NaOH solutions were collected and measured with a gamma counter to determine the unbound, membrane bound and internalized fraction of each radioactive agent.

### 1-3 in Healthy Mice

To determine the blood half-life of each Adma radioligand, freshly synthesized 1/2/3 (13.6-14.8 MBq; 1.5 nmol in 100 μL of PBS) was injected intravenously in healthy female nude mice (n=3). Blood was drawn from the saphenous vein at six different timepoints post injection (2, 5, 15, 30, 60 and 120 min). The collected blood samples were weighed and measured with a gamma counter to determine the percent injected dose per gram (% ID/g) value of each sample. In addition to studying the blood half-life of each radioligand, the renal excretion of 2 at early timepoints was studied in healthy female nude mice by collection of urine at 20 and 60 minutes post radioligand injection. Further descriptions of both experiments are detailed below.

Pretargeting with 1-3

Experimental cohorts of BxPC3 tumor bearing female nude mice (n=4-5/cohort) were injected intravenously with CB7-M5A (0.7 nmol; 100 g in 150 L in PBS) followed 72 h later with an intravenous injection of 1/2/3 (1.5 nmol; 10.2-13.9 MBq in 150 L in PBS). For pretargeting with radioligand 2, a 144 hours (“h”) lag time between the antibody and radioligand was also investigated. The cohorts were euthanized for in vivo biodistribution 4, 8 or 24 h post radioligand injection and the 24 h cohort was also imaged with a small animal PET/computer tomography (PET/CT) scanner (Siemens Inveon) at 4, 8 and 24 h post radioligand injection prior to euthanasia. An additional cohort/timepoint for pretargeted 2 was assigned, which was euthanized 2 h post radioligand injection for dosimetry calculations. The corresponding control cohort (n=4/cohort) for each ligand was injected intravenously only with 1/2/3 and sacrificed for in vivo biodistribution 24 h post injection.

### [⁸⁹Zr]Zr-DFO-M5A In Vivo Profile in Tumor Models

A cohort of BxPC3 and MIAPaCa-2 tumor bearing female nude mice (n=4/cohort) were injected intravenously with [⁸⁹Zr]Zr-DFO-M5A (0.7 nmol; 100 g; 2.3-3.4 MBq in 200 L in PBS). The mice were imaged with a small animal PET/CT scanner 72 h post injection followed by in vivo biodistribution.

Pretargeting with 2 in MIAPaCa-2 Xenografts

A cohort of MIAPaCa-2 tumor bearing female nude mice (n=4) was injected with CB7-M5A (0.7 nmol; 100 μg in 150 μL in PBS) intravenously 72 h prior to an injection of 2 (1.5 nmol; 8.8-10.1 MBq in 150 μL in PBS). The mice were imaged with a small animal PET/CT scanner 24 h post radioligand injection, followed by in vivo biodistribution.

### The Dosimetry of Pretargeted 2

The estimated dosimetry of the pretargeted 2 in an adult human male (70 kg) was calculated based on the in vivo biodistribution of the pretargeted 2 in BxPC3 tumor bearing mice. The biodistribution data was fitted using a linear interpolation between time points. The linear function of each organ was used to interpolate the concentration at intervals of 1 h to give a better estimate of the kinetics. The integration time was extended 48 h with the assumption that the % ID/organ was constant after the first 24 h and the only change in concentration between 24 and 48 h was due to radioactive decay. A trapezoidal approximation was then used to obtain the integral over the time intervals. These residence times were used to estimate the absorbed dose to a human subject using the OLINDA program¹⁶ with the adult human male model and no bladder clearance. The % ID to the large intestine was divided equally to the right and left colon for input into the OLINDA model for an adult male. The dose to the rest of the body was not used in this calculation.

### Statistical Analysis

Statistical analysis for all of the data from the biodistribution and in vitro assays was performed with 2-tailed unpaired t tests using GraphPad Prism (GraphPad Software, Inc.). A p-value between two groups of p<0.05 was considered significant. The number of mice used for each pretargeting cohort was at least four. All in vitro experiments were performed in triplicate unless otherwise noted.

### Results

### Development of the Pretargeting Agents

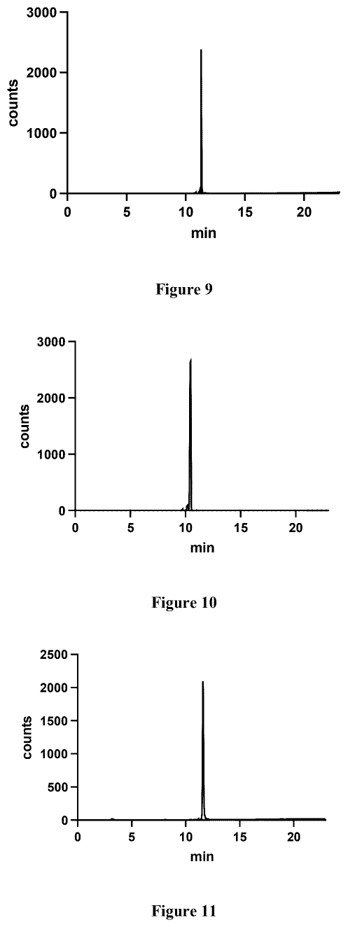

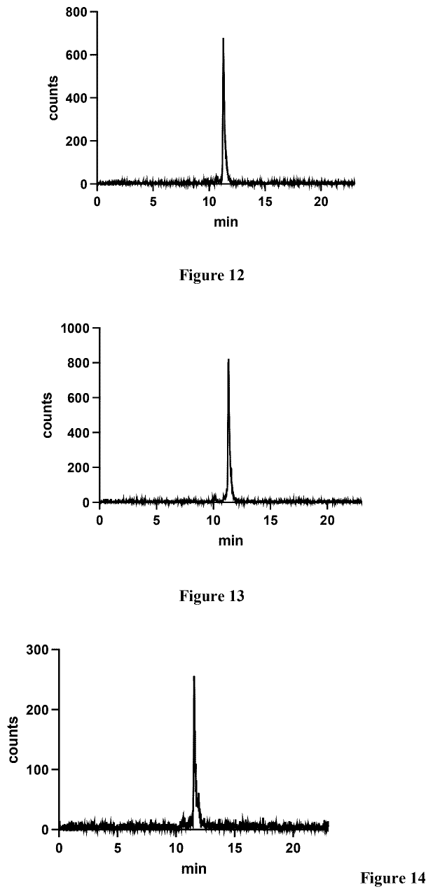

Each of the three radioligands was synthesized and radiolabeled efficiently and in good yields. Precursors for 1, 2 and 3 were synthesized with overall yields of 14.5%, 36.2% and 14.1% respectively with chemical purities of 97.6%, 97.2% and 95.8%. (FIGS. 9-11) Radioligands 1-3 were produced by radiolabeling their corresponding precursors with high radiochemical yields of 97.6±1.5%, 98.5±0.7% and 96.7±0.0% and purity of 97.5±1.0%, 98.3±1.8% and 98.9±0.7% respectively (n=3). (FIGS. 12-14)

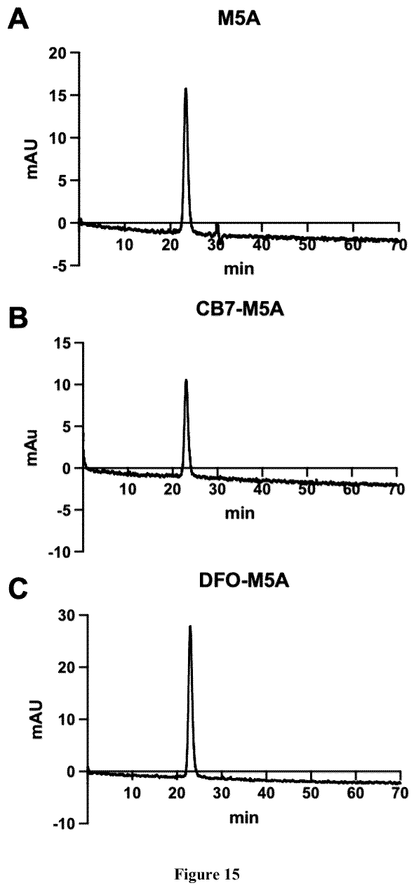

CB7 conjugated antibody was synthesized with overall recovery yield of 83%. Each M5A was determined to have 0.8±0.0 CB7 (n=3) moieties on average. Based on a quality control of the CB7-M5A, there was no aggregation or fragments present. (FIG. 15) The immunoreactivity of the CB7-conjugated antibody was 95.7±0.7% (n=3).

### In Vitro Characterization of 1-3

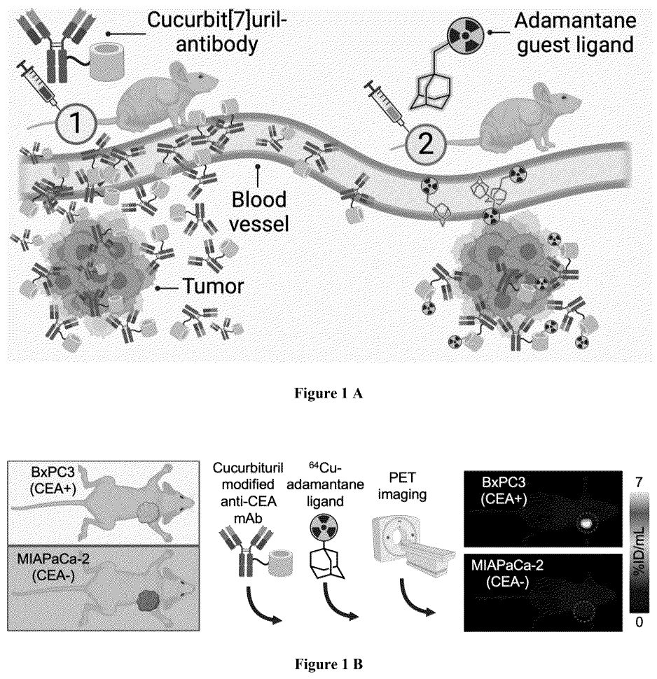

In order to compare the three radioligands, in vitro analysis was performed on each of them to determine their relative pharmacological characteristics and suitability for in vivo pretargeting experiments. 1-3 demonstrated great in vitro stability in PBS and in bovine plasma. (FIG. 1B, Tables 1-2) At 24 h, 95.8±2.2%, 99.0±0.8% and 95.9±3.1% of 1, 2 and 3 was still intact in PBS and 95.2±0.9%, 90.6±1.6% and 97.5±0.6% of 1, 2 and 3 remained intact in bovine plasma samples. No free copper-64 was observed in any of the stability samples, suggesting that the radioligands were sufficiently stable for the application. At 1 h timepoint, the Adma-radioligands exhibited decreased plasma binding as the length of polyethylene glycol (PEG) linker increased (23.8±6.6 (1); 14.0±5.4 (2); 8.1±2.0 (3)). Additionally, the fraction of protein bound radioligand grew overtime to the last timepoint of 24 h (27.8±2.1(1); 30.5±3.7 (2); 23.2±2.1 (3)) These differences were determined to be not significant amongstthe molecules (p>0.05).

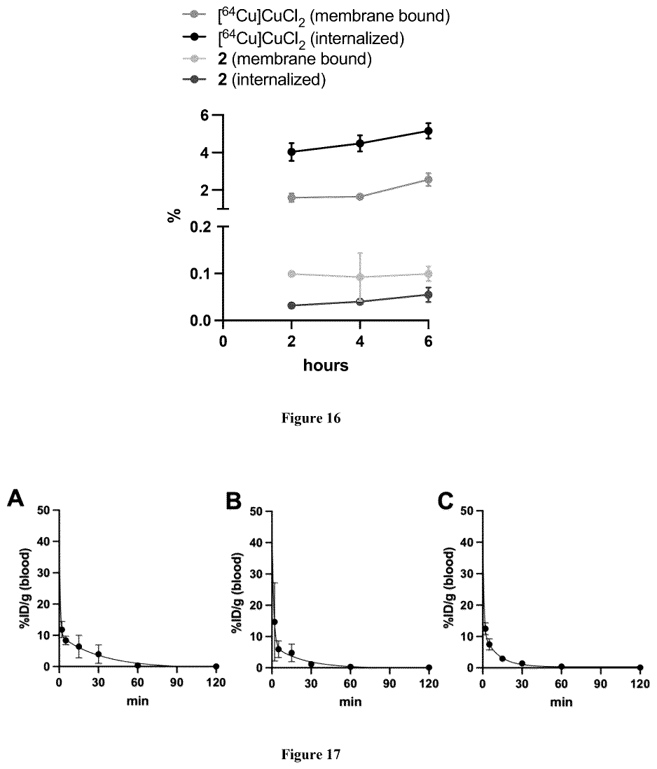

Based on the cell internalization assay in BxPC3 cells, 2 did not bind to the cell membrane (0.1±0.1%) or get internalized (0.0±0.0%) even after a 6 h incubation period. (FIG. 16) [⁶⁴Cu]CuCl₂ demonstrated significantly higher internalization (5.2±0.3%; p=0.002) compared to 2 confirming that copper-64-NOTA complex of 2 remained intact for the duration of the experiment.

### Development of the DFO-Modified Antibody

Based on the quality control, the DFO conjugated M5A antibody was intact and no fragments or aggregates were detected. (FIG. 15) The overall recovery yield was 85%. The immunoreactive fraction of the DFO-M5A was measured to be 89.6±2.1%, which was suitable for in vivo analysis.

### 1-3 in Healthy Mice

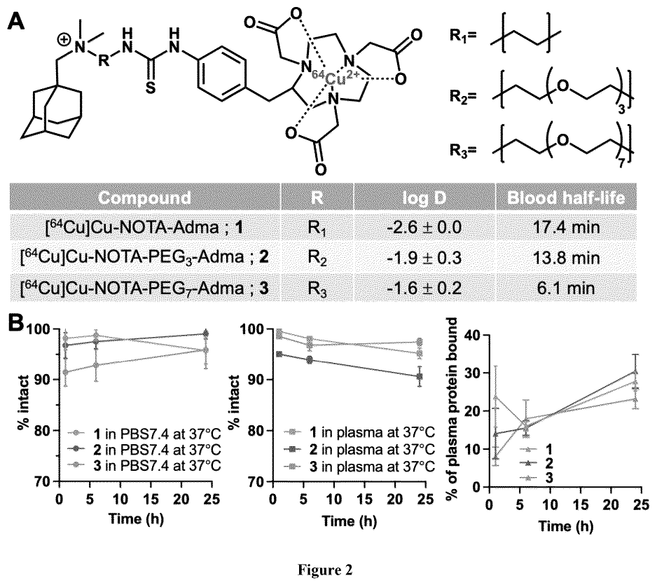

In order to determine the route and relative rate of clearance of the radioligands, their blood half-life was investigated. Additionally, because a combination of both renal and hepatobiliary clearance was expected, urine at time points earlier than the first biodistribution time point was collected. The in vivo blood half-life experiments revealed that the blood half-life had a negative correlation with the number of PEG-units incorporated to the radioligand. (FIGS. 2A,17) The ligands' blood half-life decreased as the PEG-linker length increased (1: 17.4 min; 2: 13.8 min; 3: 6.1 min). Based on the urine samples at 20 min and 60 min post injection of 2 into healthy mice, the radioligand demonstrated high renal clearance at these early timepoint, 680±210 and 1050±450% ID/g respectively. (Table 3)

### In Vivo Biodistribution of Pretargeted 1-3

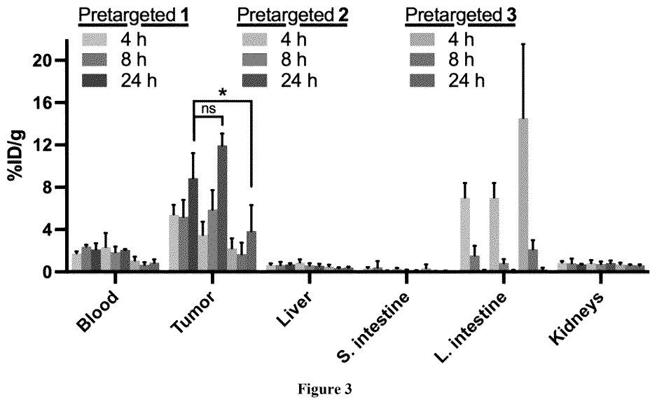

In vivo biodistribution studies were performed in order to assess the relative effectiveness of the three radioligands for pretargeting of tumors in a mouse model. Of the three Adma radioligands that were studied with a 72 h lag time schedule, only pretargeted 1 and 2 demonstrated significantly higher tumor uptake compared to their respective control cohorts (1: p=0.005 & 2: p=0.003, Tables 4-5). Pretargeted 3 resulted in almost 4 times higher average tumor uptake compared to control (3.9±2.1% ID/g & 0.0±0.0% ID/g; Table 6). However, the tumor uptake between the cohorts was not significant (p=0.053). The tumor uptake increased over time in all the cohorts, yet the highest average tumor uptake was obtained with pretargeted 2 (1: 8.9±2.0% ID/g & 2: 12.0±0.9% ID/g & 3: 3.9±2.1% ID/g). (FIG. 3) To demonstrate that pretargeting platform may be suitable for clinical applications without the use of clearing agents, the best performing radioligand (2) with a 144 h lag time was explored, which is more aligned with the biological half-life of mAbs in humans. For the pretargeted 2, the tumor uptake was lower when it was administered 144 h compared to 72 h post CB7-M5A injection (144 h: 5.3±1.4% ID/g; p=0.01) but tumor to blood uptake ratio was substantially higher for the 144 h cohorts. (Tables 7, 8)

The presence of all the Adma-radioligands in the blood pool was significantly higher in the pretargeting cohorts compared to their corresponding control cohorts (p=0.001, p=0.0006 and p=0.01 respectively for 1, 2 and 3 with 72 h lag time) revealing that the Adma radioligands bound to the remaining CB7-M5A circulating in the blood. However, when the lag time was extended from 72 to 144 h, the presence of the pretargeted 2 in the blood was not significantly higher compared to controls studies when only 2 was administered (p=0.13) suggesting that less of 2 was binding to the CB7-M5A in the blood with the longer lag time. (Table 7) The lower presence of 2 bound CB7-M5A circulating in the blood pool likely contributed to the tumor uptake of pretargeted 2 being lower at later timepoints with the extended lag time. Initially at 4 h timepoint, the tumor uptake of pretargeted 2 is similar with 72 and 144 h lag times (3.5±1.1% ID/g and 3.2±1.4% ID/g respectively).

All three pretargeted radioligands excreted through the kidneys/bladder and gastrointestinal tract. With a 72 h lag time, the excretion was the slowest for pretargeted 3. For pretargeted 2, the excretion through the intestine suggested to be slower with a longer lag time of 144 h. However, the standard deviation on the % ID/g values are large for the organ since feces was not removed from the large intestine samples, which could partly explain the difference between the lag time cohorts. By 24 h post injection the excretion was complete for 1-3. Despite the low kidney retention at 4 h with pretargeted 1-3 (<1.0% ID/g; Table 4-7), 2 demonstrated early renal clearance in healthy mice. (Table 3)

As expected, tumor-to-blood ratios increased over time with the pretargeted ligands. (Table 8) The difference between the first and last timepoint was significant only with pretargeted 2 with 72 h lag time (4 h: 2.3±1.5 & 24 h: 5.8±0.4; p=0.008). The low presence of 2 in the blood pool with the 144 h lag time resulted in the highest tumor-to-blood ratio at 24 h post injection out of all the studied conditions (16.7±4.6).

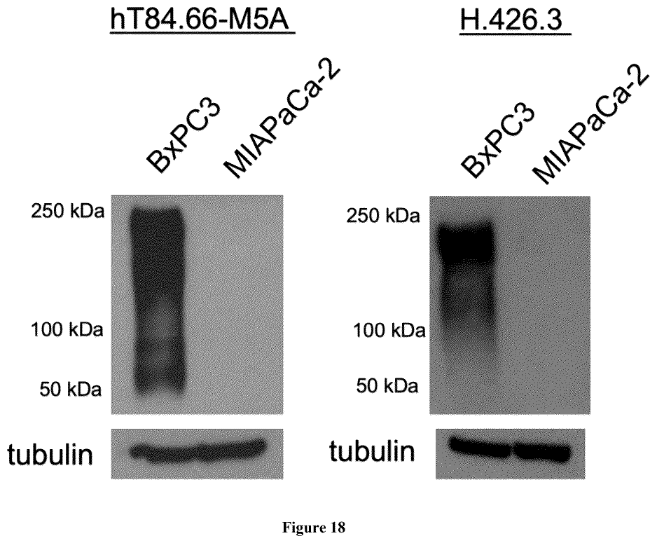

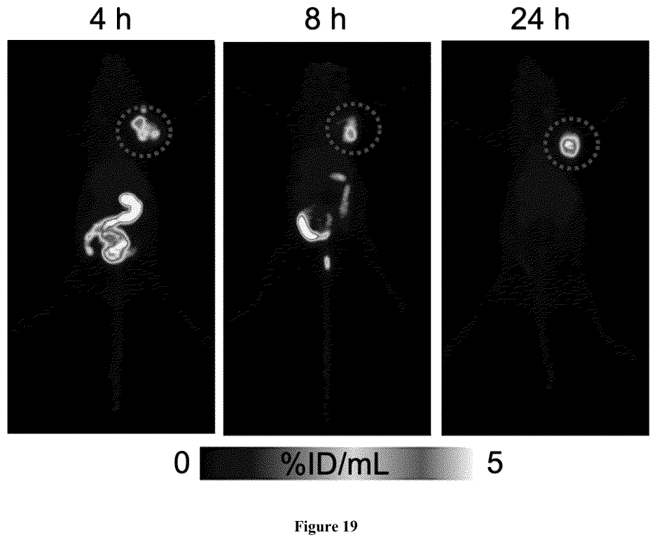

As it was expected based on the CEA expression differences in BxPC3 and MIAPaCa-2 cells (FIG. 18), the tumor uptake of the pretargeted 2 was significantly lower in MIAPaCa-2 tumor models compared to BxPC3 (0.5±0.1 vs. 12.0±0.9% ID/g; p=0.003), demonstrating the specificity of the interaction of the radioligands with the tumors. (FIG. 5D)

### Pretargeted PET 1-3

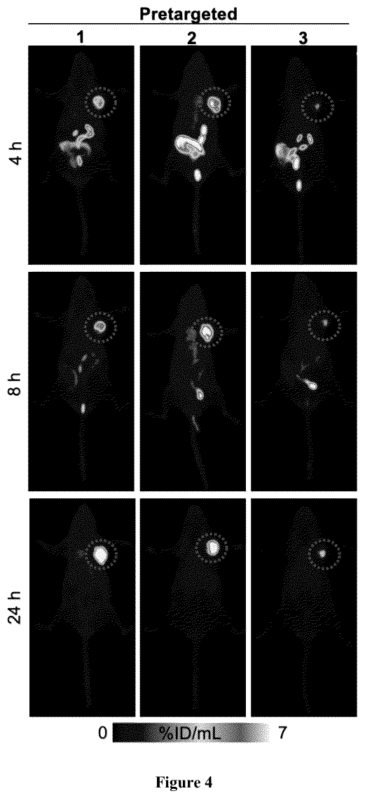

The PET imaging of the pretargeted 1, 2 and 3 in BxPC3 tumor bearing nude mice resulted in successful delineation of the tumor mass at all timepoints. (FIGS. 4,19) The images confirmed the in vivo biodistribution data, showing that the tumor uptake and tumor-to-background signal increased overtime up to the last timepoint. The tumor uptake of the pretargeted 1-3 varied between 3.8-17.1; 14.5-24.0; 2.8-13.1; % ID/mL respectively with the 72 h lag time and 3.8-7.9% ID/mL with the 144 h lag time at 24 h post radioligand injection. For the pretargeted 2 in MIAPaCa-2 tumor bearing mice, the tumor uptake values were between 0.8-1.4% ID/mL, indicating that there was no specific uptake in the tumors due to the lack of CEA expression. (FIG. 5B)

### In Vivo Profile of [⁸⁹Zr]Zr-DFO-M5A in Xenograft Models

To compare the uptake in antigen expressing and antigen negative cell lines with M5A, directly-labeled [⁸⁹Zr]Zr-DFO-M5A was used. The in vivo profile of the [⁸⁹Zr]Zr-DFO-M5A in the subcutaneous tumor mouse models of BxPC3 and MIAPaCa-2 revealed varying tumor uptakes between the models. The findings aligned with the Western Blotting results on the CEA expression of the cell lines as well as previously published results from with the same cell lines (Girgis, 2011, Yunis, 1977 and Tan, 1986). (FIG. 18) The CEA-positive BxPC3 xenografts demonstrated higher tumor uptake compared to the CEA-negative xenografts of MIAPaCa-2 (53.7±10.0 vs 7.3±0.3% ID/g; p=0.004). (FIG. 5C) Based on these imaging experiments, the tumor-to-background signal was higher in BxPC3 cohort compared to MIAPaCa-2 and the tumor uptake values in both tumor models supported the findings of the in vivo biodistribution studies (BxPC3: 50.4±8.9% ID/mL & MIAPaCa-2: 4.8±0.2% ID/g. (FIG. 5A)

### Dosimetry of the Pretargeted 2

To determine the dosimetric advantages of the pretargeting method, the dosimetry was calculated and compared it to that of the directly labeled [⁸⁹Zr]Zr-DFO-M5A. The estimated dose of an adult male for pretargeted 2 demonstrated that the CB7-Adma pretargeting approach resulted in much lower organ doses compared to the use of [⁸⁹Zr]Zr-DFO-M5A, whose dosimetry analysis was reported earlier by previous study (Jallinoja, 2022). (Table 9) In all the investigated organs, the dose of the pretargeted 2 was reduced several fold compared to the directly radiolabeled M5A. The dose limiting organ for the use of directly radiolabeled antibodies is often red marrow and here its dose with [⁸⁹Zr]Zr-DFO-M5A demonstrated to be almost 70-fold higher compared to that of the pretargeted 2. The reported injected clinical doses of zirconium-89 labeled mAbs vary between 37-185 MBq (Ulaner 2017, Der Houven, 2015). In this regard, with an estimated 110 MBq clinical dose of [⁸⁹Zr]Zr-DFO-M5A, the effective dose would be 95.5 mSv. A clinical dose of 150 MBq of pretargeted 2 however would give rise to a lower effective dose of 6.0 mSv while still providing the necessary tumor to non-target tissue uptake ratios to clearly delineate CEA-expressing tumors.

### Discussion

The present invention has demonstrated here that host-guest CB7-Adma pretargeted PET results in specific and exceptionally high tumor uptake and tumor-to-background signal. (FIG. 3, Table 4-8) The efficacy of the pretargeting methodology was evaluated with three copper-64-labeled Adma-radioligands. The pretargeted radioligand with the best in vivo profile, 2, was also studied with an additional lag time. Since the motivation for the studies was the development of a pretargeting platform, the dosimetry of a directly radiolabeled [⁸⁹Zr]Zr-DFO-M5A and CB7-M5A pretargeted 2 was compared. (Table 9) While the isotopes were not matched for these experiments, [⁸⁹Zr]Zr-DFO-M5A was chosen because zirconium-89 is the most clinically relevant isotope for PET imaging with mAbs and it provides information on the biological fate of the mAbs at 5-7 days post injection whereas copper-64 based PET with mAbs is only useful for ~2 days post injection when there is still very low tumor to non-target tissue uptake, especially in the blood.

The difference between 1-3 was observed in their respective blood half-lives and log D values, which resulted in the ligands having variance in their tumor uptake and clearance profile. The number of PEG-units incorporated to the Adma radioligand correlated negatively with the compounds log D value and blood half-life. The same correlation between the number of PEG-units and the log D values was observed earlier with ferrocene radioligands designed for pretargeting (Jallinoja, 2022). As has been reported earlier, with higher number of hydrophilic PEG-units the expectation is to have lower respective log D value (Meyer, 2017).

In turn, the log D values of 1-3 negatively correlated with the blood half-life values. This trend of more hydrophilic compounds diffusing from the blood stream through the cell membrane at a lower rate resulting in longer blood half-life has been established before (Obach, 2008, Lewis, 2019). The difference in tumor uptake of pretargeted 1 and 2 was not significant, despite their relative difference in the blood half-life. However, the Adma-radioligand with the shortest blood half-life, 3, resulted in lowest tumor uptake, which was determined to be significant compared to pretargeted 1 and 2. This positive correlation between blood half-life and tumor uptake has been reported earlier by us and other laboratories (Jallinoja, 2022, Meyer 2017). As the radioligand remains in the blood pool longer, the molecule has more time to accumulate at the target site.

M5A demonstrated high specificity towards the CEA, which was validated with in vivo experiment with directly labeled M5A ([⁸⁹Zr]Zr-DFO-M5A) and pretargeted 2 in CEA-positive (BxPC3) and CEA-negative (MIAPaCa-2) tumor bearing mice. (FIG. 5) Both imaging experiments in the two tumor models showed that the high specificity and excellent tumor-to-background signal which antibody-based imaging provides is not lost when the antibody imaging is done with the CB7-Adma pretargeting strategy. The obtained tumor-to-blood ratio of pretargeted 2 at 24 h post injection with the longer lag time schedule of 144 h was higher compared to that of [⁸⁹Zr]Zr-DFO-M5A at 72 h post injection (16.7±4.6 and 7.5±1.1 respectively). (Tables 8,9) Interestingly, the fact that it was able to obtain excellent tumor targeting with a 144 h lag time suggests that even longer lag times may still be possible, which would further improve the dosimetry by reducing exposure in non-target tissues including the blood and marrow. This would potentially improve the image contrast using described pretargeted approach in this invention as well as improve the therapeutic index when turning this strategy to targeted radionuclide therapy.

The use of ferrocene radioligand driven host-guest chemistry methodology for pretargeted PET was previously reported (Jallinoja 2021, Jallinoja, 2022). Comparing the CB7-ferrocene pair to CB7-Adma pair as the pretargeting agents, there are several advantages in the latter approach. All the investigated Adma-radioligands maintained high in vitro stability in plasma up to 24 h, unlike the reported ferrocene radioligands (70-80% intact at 4 h). Additionally, the formed Adma-CB7 host-guest complex exhibited higher in vivo stability compared to the previously reported CB7-ferrocene complex. Increasing tumor uptake was observed in pretargeting experiments with 1-3 from the first studied timepoint (4 h) to the last (24 h) whereas the opposite trend is observed in the CB7-ferrocene pretargeting approach and the dissociation of the ferrocene radioligand from the CB7 over time is observed. All in all, using the same antibody, pretargeting schedule, dosing and mouse tumor model, the highest tumor uptake values experienced with lead Adma radioligand candidate (2) are several times higher (8 h: 5.9±1.6% ID/g & 24 h: 12.0±0.9 ID/g) compared to the previously reported ferrocene radioligand (8 h: 3.1±0.6% ID/g & 24 h: 1.5±0.5% ID/g) (Jallinoja 2022). The in vivo stability of a preformed CB7-Adma complex has been explored earlier with carbon-11 and fluorine-18 labeled Adma guests in rodents (Strebl 2018). The complexes exhibited high stability in vivo, however, the stability was studied only up to 1 h. This invention expands the knowledge on the in vivo formed host-guest complexes and their stability.

It was hypothesized that one major advantage of host-guest based pretargeting lies in the high stability of the CB7-Adma components which allows to perform pretargeting with flexible pretargeting dosing schedules and preferably longer lag times (>3 days). The reported pretargeting strategy excels with varying lag times making the strategy clinically approachable. Importantly the feasibility of longer lag times potentially allows the methodology to further decrease the whole-body radiation dose and increase tumor-to-background signal, which is the motivation for shifting to pretargeting technologies from the use of directly radiolabeled antibodies.

The present invention describes the development and characterization of three Adma-radioligands for CB7-Adma host-guest pretargeted PET. The use of copper-64-labeled Adma radioligand along with a CB7-modified anti-CEA full-length antibody as the pretargeting agent pair for pretargeted PET in BxPC3 tumor bearing nude mice resulted in high, long remaining target uptake of the radioligand. This is the first reported pretargeting study utilizing the CB7-Adma complex formation as the pretargeting interaction for antibody-based PET.

The adamantane radioligands possessed high in vitro stability up to 24 h (>90%). Pretargeted positron emission tomography with cucurbit[7]uril-adamantane methodology resulted in specific tumor uptake (p<0.05) with low background signal. The in vivo formed cucurbit[7]uril-adamantane complex was demonstrated to be stable with high tumor uptake up to 24 h post radioligand injection (12.0±0.9% ID/g). The total body radiation dose of the pretargeting strategy was only 3.3% of that of the directly zirconium-89-labeled hT84.66-M5A. The cucurbit[7]uril-adamantane strategy is highly suitable for pretargeted positron emission tomography. The exceptional stability of the pretargeting agents and the specific and high tumor uptake of the pretargeted adamantane radioligands provides great potential for the platform.

### REFERENCES

1. Altai M, Membreno R, Cook B, Tolmachev V, Zeglis BM. Pretargeted Imaging and Therapy. J Nucl Med. 2017; 58(10):1553-9.
2. Jallinoja V, Houghton JL. Current Landscape in Clinical Pretargeted Radioimmunoimaging and Therapy. J Nucl Med. 2021.
3. Assaf K I, Nau W M. Cucurbiturils: from synthesis to high-affinity binding and catalysis. Chem Soc Rev. 2015; 44(2):394-418.
4. Wanka L, Iqbal K, Schreiner P R. The lipophilic bullet hits the targets: medicinal chemistry of adamantane derivatives. Chem Rev. 2013; 113(5):3516-604.
5. Shetty D, Khedkar J K, Park K M, Kim K. Can we beat the biotin-avidin pair?: cucurbit[7]uril-based ultrahigh affinity host-guest complexes and their applications. Chem Soc Rev. 2015; 44(23):8747-61.
6. Zhao X B, Kang J Y, Shi Y P. Noncovalent Dual-Locked Near-Infrared Fluorescent Probe for Precise Imaging of Tumor via Hypoxia/Glutathione Activation. Anal Chem. 2022; 94(17):6574-81.
7. Sembo-Backonly B S, Estour F, Gouhier G. Cyclodextrins: promising scaffolds for MRI contrast agents. RSC Adv. 2021; 11(47):29762-85.
8. Wu H, Chen Z, Qi S, Bai B, Ye J, Wu D, et al. Evaluation of the stability of cucurbit[8]uril-based ternary host-guest complexation in physiological environment and the fabrication of a supramolecular theranostic nanomedicine. J Nanobiotechnology. 2021; 19(1):330.
9. Strebl M G, Yang J, Isaacs L, Hooker J M. Adamantane/Cucurbituril: A Potential Pretargeted Imaging Strategy in Immuno-PET. Mol Imaging. 2018; 17:1536012118799838.
10. Girgis M D, Olafse T, Kenanova, V, McCabe K E, Wu A M, Tomlinson J S. Targeting CEA in Pancreas Cancer Xenografts with a Mutated scFv-Fc Antibody Fragment. EJNMMI Res. 2011; 1:24.
11. Yunis A A, Arimura G K, Russin D J. Human pancreatic carcinoma (mia paca-2) in continuous culture: Sensitivity to asparaginase. Int. J. Cancer. 1977; 19:128-135.
12. Tan M H, Nowak N J, Loor R, Ochi H, Sandberg A A, Lopez C, Pickren J W, Berjian R, Douglass H O, Chu M T. Characterization of a New Primary Human Pancreatic Tumor Line. Cancer Investigation. 1986; 4:15-23.
13. Jallinoja V I J, Carney B D, Zhu M, Bhatt K, Yazaki P J, Houghton J L. Cucurbituril-Ferrocene: Host-Guest Based Pretargeted Positron Emission Tomography in a Xenograft Model. Bioconjug Chem. 2021; 32(8):1554-8.
14. Jallinoja V I J, Carney B D, Bhatt K, Abbriano C H, Schlyer D J, Yazaki P J, et al. Investigation of Copper-64-Based Host-Guest Chemistry Pretargeted Positron Emission Tomography. Mol Pharm. 2022.
15. Lindmo T, Bunn P A, Jr. Determination of the true immunoreactive fraction of monoclonal antibodies after radiolabeling. Methods Enzymol. 1986; 121:678-91.
16. Stabin M G, Sparks R B, Crowe E. OLINDA/EXM: the second-generation personal computer software for internal dose assessment in nuclear medicine. J Nucl Med. 2005; 46(6):1023-7.
17. Ulaner G A, Hyman D M, Lyashchenko S K, Lewis J S, Carrasquillo J A. 89Zr-Trastuzumab PET/CT for Detection of Human Epidermal Growth Factor Receptor 2-Positive Metastases in Patients With Human Epidermal Growth Factor Receptor 2-Negative Primary Breast Cancer. Clin Nucl Med. 2017; 42:912-917.
18. der Houven C. Menke-van, Gootjes E C, Huisman M C, Vugts D J, Roth C, Luik A. Marije, et al. 89Zr-cetuximab PET imaging in patients with advanced colorectal cancer. Oncotarget. 2015; 6:30384-30393.
19. Meyer J P, Kozlowski P, Jackson J, Cunanan K M, Adumeau P, Dilling T R, et al. Exploring Structural Parameters for Pretargeting Radioligand Optimization. J Med Chem. 2017; 60(19):8201-17.
20. Obach R S, Lombardo F, Waters N J. Trend analysis of a database of intravenous pharmacokinetic parameters in humans for 670 drug compounds. Drug Metab Dispos. 2008; 36(7):1385-405.
21. Lewis J S, Windhorst A D, Zeglis B M. Radiopharmaceutical Chemistry. Springer Cham; 2019: 125-127.

1. A compound having the structure: 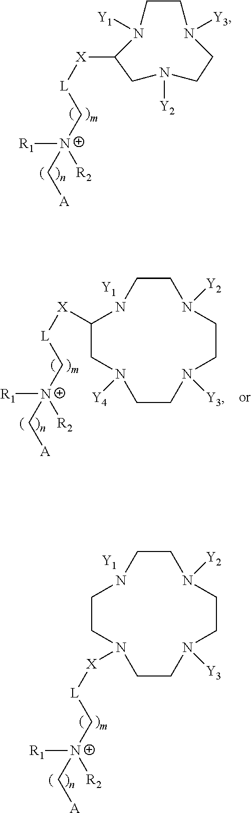
wherein Y₁, Y₂, Y₃, Y₄ are each, independently, —H, alkyl-N—(CO₂R₄)₂, alkyl-N-(alkyl-CO₂R₄)₂, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ or alkylheteroaryl-P(O)(OH)₂, and
wherein each occurrence of R₄ is independently, —H, —OH, —NH₂, halogen, alkyl, —O-alkyl, —NH— alkyl, —CHF₂, —CF₃, —OCHF₂, —OCF₃, amide, alkenyl, alkynyl, alkyl-aryl, alkyl-heteroaryl, aryl, heteroaryl, alkyl-CF₃, or —Si(alkyl)₃; preferably, R₄ is —OH, —NH₂, —O—(C₁-C₆ alkyl), or NH—(C₁-C₆ alkyl), more preferably R₄ is —OH or —NH₂;
wherein X is alkyl-aryl-thiourea, alkyl-heteroaryl-thiourea, alkyl-cycloalkyl-thiourea, alkenyl-aryl-thiourea, alkenyl-heteroaryl-thiourea, alkenyl-cycloalkyl-thiourea, alkynyl-aryl-thiourea, alkynyl-heteroaryl-thiourea, or alkynyl-cycloalkyl-thiourea;
wherein L is a chemical linker;
wherein R₁ and R₂ are each independently H, halogen, alkyl, alkenyl, alkynyl, —OH, —O-(alkyl), —CHF₂, —CF₃, —OCHF₂ or —OCF₃;
wherein n and m are each independently 0, 1, 2, 3, 4, 5, or 6; and
wherein A is a guest molecule which is substituted or unsubstituted adamantane, ferrocene, diamantane, 4,9-diamino diamantane, bicyclo[2.2.2]octane, iceane, triamantane, isotetramantane, pentamantane, cyclohexamantane, super-adamantane, 1,3,5,7-tetramethyl-1,3,5,7-tetrasilaadamantane, adamanzane, antimony trioxide, arsenic trioxide, 2,4,6-trioxa-1,3,5,7-tetraarsaadamantane, diamondoid, hexamethylenetetramine, phosphorus pentasulfide, phosphorus pentoxide, phosphorus trioxide, tetramethylenedisulfotetramine, tetrodotoxin, or 1,3,5-Triaza-7-phosphaadamantane;
or salt or ester thereof.

2. The compound of claim 1, wherein the compound is other than

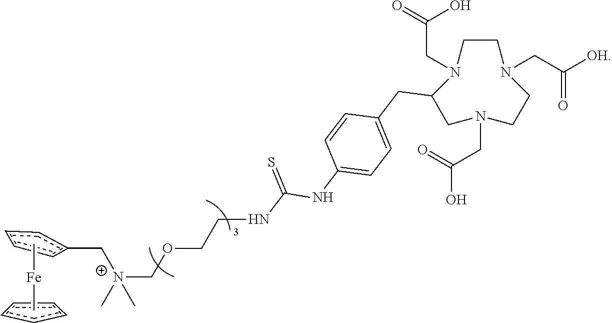

3. The compound of claim any one of claims 1-2, wherein Y₁, Y₂, Y₃, Y₄ are each independently alkyl-CO₂H, alkylaryl-CO₂H, alkyl-N—(CO₂R₄)₂, alkyl-N-(alkyl-CO₂R₄)₂, alkylheteroaryl-CO₂H, alkyl-CO₂R₄, alkylaryl-NH—CO₂R₄, alkylaryl-CO₂R₄, alkylheteroaryl-CO₂R₄, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂; preferably, Y₁, Y₂, Y₃, Y₄ are each independently alkyl-CO₂H, alkyl-N—(CO₂R₄)₂, alkyl-N-(alkyl-CO₂R₄)₂, alkylaryl-CO₂H, alkylheteroaryl-CO₂H, alkyl-N(alkylaryl-CO₂H)₂, alkyl-N(alkylheteroaryl-CO₂H)₂, alkyl-N(alkyl-CO₂H)₂, alkyl-N(alkylaryl-OH)(alkyl-CO₂H), alkyl-N(alkylheteroaryl-OH)(alkyl-CO₂H), or alkylheteroaryl-P(O)(OH)₂; more preferably, Y₁, Y₂, Y₃, Y₄ are each independently alkyl-CO₂H, alkyl-N—(CO₂R₄)₂, alkyl-N-(alkyl-CO₂R₄)₂, or alkyl-N(alkyl-CO₂H)₂; more preferably, Y₁, Y₂, Y₃, Y₄ are each independently alkyl-CO₂H, CH₂—N—(CO₂H)₂, or CH₂—N-(alkyl-CO₂R₄)₂.

4. The compound of any one of claims 1-3, wherein at least two of Y₁, Y₂ and Y₃ are same; preferably, Y₁, Y₂ and Y₃ are same; or wherein at least two of Y₁, Y₂, Y₃ and Y₄ are same; preferably, at least three of Y₁, Y₂, Y₃ and Y₄ are same; more preferably, Y₁, Y₂, Y₃ and Y₄ are same.

5. The compound of any one of claims 1-4, wherein X is alkyl-aryl-thiourea, alkyl-heteroaryl-thiourea, alkyl-cycloalkyl-thiourea, alkenyl-aryl-thiourea, or alkenyl-heteroaryl-thiourea, preferably, X is alkyl-aryl-thiourea, alkyl-heteroaryl-thiourea, or alkyl-cycloalkyl-thiourea, more preferably, X is alkyl-aryl-thiourea, or alkyl-heteroaryl-thiourea, most preferably, X is alkyl-aryl-thiourea.

6. The compound of any one of claims 1-5, wherein the chemical linker L is an alkyl, alkenyl, alkynyl, alkylether, alkylthioether, alkylamino, alkylamido, alkylester, alkylaryl, alklyheteroaryl, polyethylene glycol (PEG), aryl, heteroaryl, a natural amino acid, an unnatural amino acid, a disulfide or thioether containing linker or combinations thereof; preferably, the chemical linker L is an alkyl linker, an alkyne linker, alkynal linker or a polyethylene glycol (PEG) or combinations thereof, preferably, the chemical linker L is an alkyl or a PEG or combinations thereof, more preferably, the chemical linker L is a PEG.

7. The compound of any one of claims 1-6, wherein R₁ and R₂ are each independently H, halogen, C₁-C₆ alkyl, C₁-C₆ alkenyl, or C₁-C₆ alkynyl; preferably, R₁ and R₂ are each independently, C₁-C₆ alkyl or C₁-C₆ alkenyl; more preferably, R₁ and R₂ are each independently C₁-C₆ alkyl; more preferably, R₁ and R₂ are each independently C₁₋₅ alkyl, more preferably, R₁ and R₂ are each independently C₁₋₃ alkyl, more preferably, R₁ and R₂ are ethyl; more preferably, R₁ and R₂ are methyl.

8. The compound of any one of claims 1-7, wherein n and m are each independently 0, 1, 2, 3, 4, 5, or 6; preferably, n and m are each independently 0, 1, 2, or 3; more preferably, n and m are each independently 0, or 1.

9. The compound of any one of claims 1-8, wherein the guest molecule A is substituted or unsubstituted adamantane, diamantane, 4,9-diamino diamantane, ferrocene, bicyclo[2.2.2]octane, buckminsterfullerene (C60), iceane, triamantane, isotetramantane, ferrocene-modified peracetic acid, pentamantane, or cyclohexamantane; more preferably, the guest molecule A is adamantane, ferrocene, 4,9-diamino diamantane, bicyclo[2.2.2]octane, iceane, diamantane, triamantane, isotetramantane, pentamantane, or cyclohexamantane, more preferably, the guest molecule A is adamantane, 4,9-diamino diamantane, or ferrocene, most preferably, the guest molecule A is adamantane.

10. The compound of any one of claims 1-9, wherein
Y₁, Y₂, Y₃, Y₄ are each independently —H, 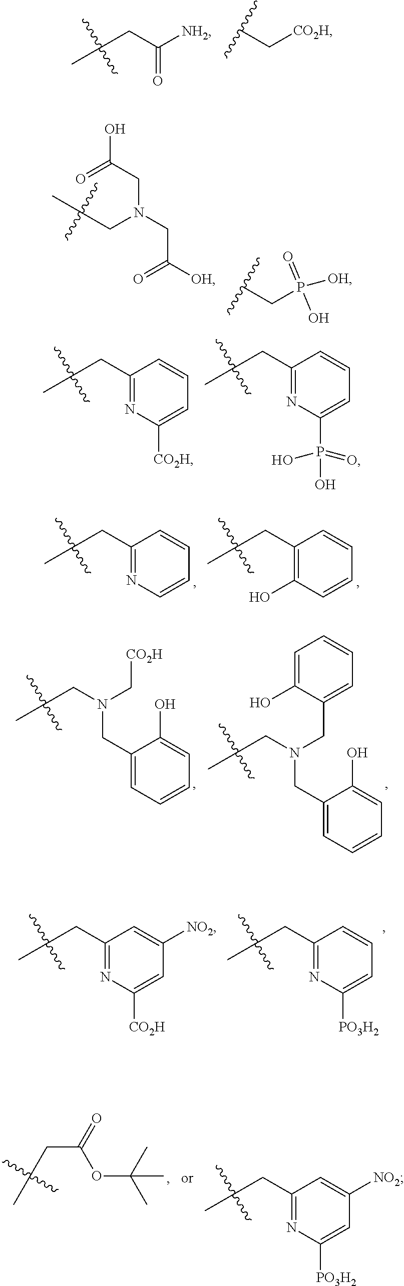
preferably Y₁, Y₂, Y₃, Y₄ are each independently 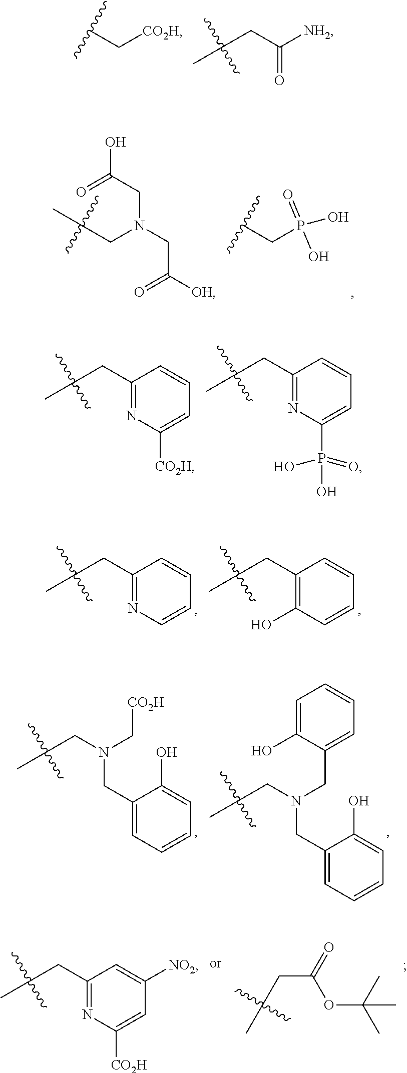
more preferably, Y₁, Y₂, Y₃, Y₄ are each independently 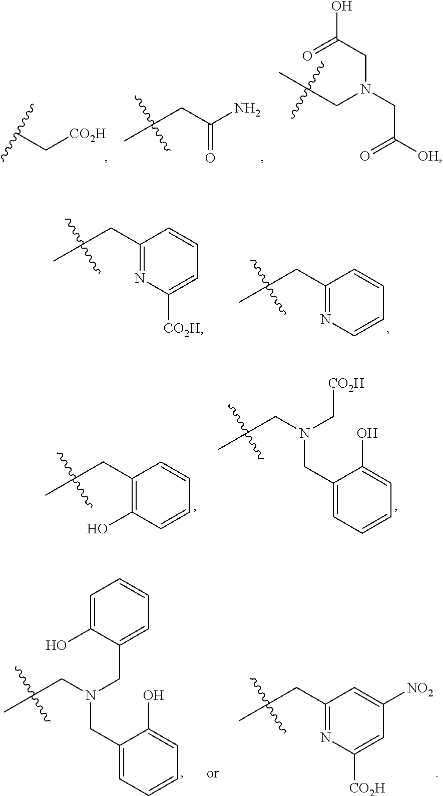
more preferably, Y₁, Y₂, Y₃, Y₄ are each independently 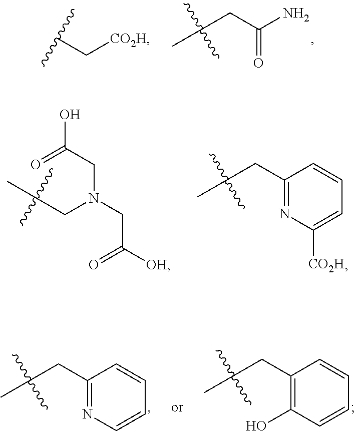 [SMILES: *CC(=O)O ; *CC(N)=O ; *CN(CC(=O)O)CC(=O)O ; *Cc1cccc(C(=O)O)n1 ; *Cc1ccccn1 ; *Cc1ccccc1O]
more preferably, Y₁, Y₂, Y₃, Y₄ are each independently 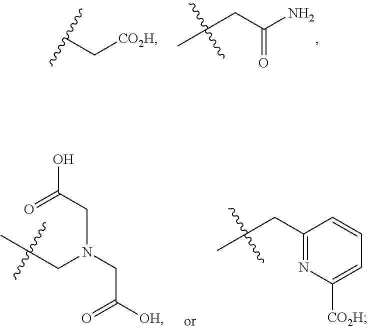 [SMILES: *CC(=O)O ; *CC(N)=O ; *CN(CC(=O)O)CC(=O)O ; *Cc1cccc(C(=O)O)n1 ; "or"]
more preferably, Y₁, Y₂, Y₃, Y₄ are each independently

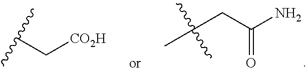 [SMILES: *CC(=O)O ; *CC(N)=O ; "or" ; "."]

11. The compound of any one of claims 1-9 having the structure: 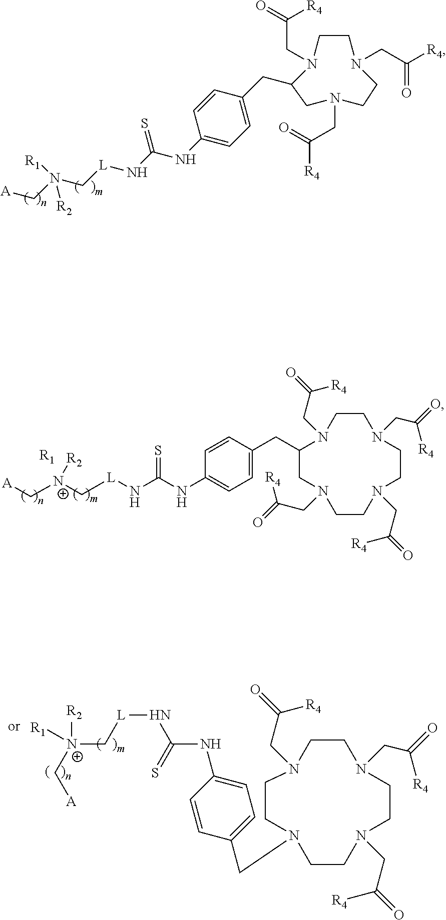
wherein R₄ is H, halogen, C₁-C₆ alkyl, —OH, —O—(C₁-C₆ alkyl), —NH—(C₁-C₆ alkyl), —CHF₂, —CF₃, —OCHF₂, or —OCF₃; preferably, R₄ is —OH, —NH₂, —O—(C₁-C₆ alkyl), or NH—(C₁-C₆ alkyl), more preferably R₄ is —OH or —NH₂.

12. The compound of any one of claims 1-9, wherein the compound having the structure: 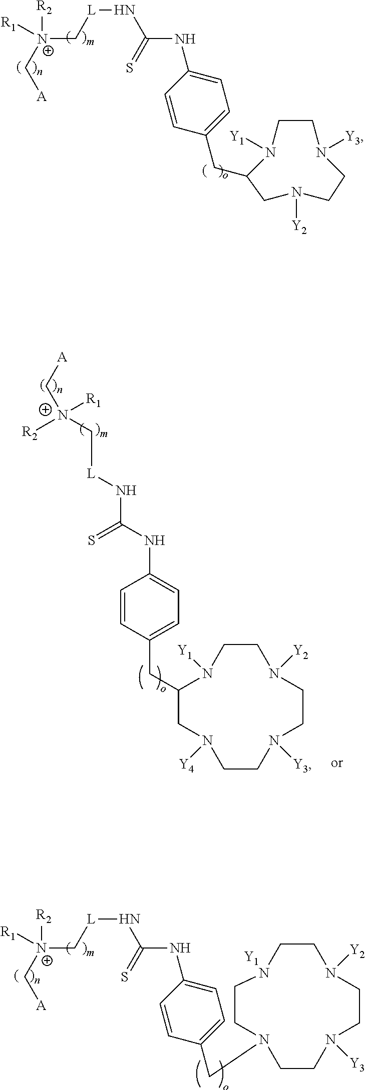
wherein n and m are each independently 0, 1, 2, 3, 4, 5, or 6; preferably, n and m are each independently 1, 2, or 3; more preferably, n and m are 1;
wherein o is 0, 1, 2, 3, 4, 5, or 6; preferably o is 1, 2, or 3; more preferably, o is 1.

13. The compound of any one of claims 1-12, wherein
a) alkyl is C₁₋₆ alkyl, preferably, alkyl is C₁₋₃ alkyl, more preferably, alkyl is methyl or ethyl; more preferably alkyl is methyl; and/or
b) aryl is phenyl, p-toluenyl (4-methylphenyl), naphthyl, tetrahydronaphthyl; indanyl, biphenyl, phenanthryl, anthryl or acenaphthyl, preferably, aryl is phenyl, p-toluenyl (4-methylphenyl), or naphthyl, more preferably, aryl is phenyl.

14. The compound of claim 6, wherein the chemical linker L has the following structure: 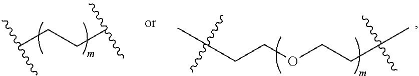
wherein m is 1, 2, 3, 4, 5, 6, 7, 8, 9, or 10; preferably m is 1, 2, 3, 4, 5, 6, or 7; more preferably, m is 1, 3, or 7.

15. The compound of claim 9, wherein the substituted ferrocene is substituted with C₁-C₆ alkyl, -alkyl-N—(C₁-C₆ alkyl), —OH, —O—(C₁-C₆ alkyl), —NH—(C₁-C₆ alkyl), —CHF₂, —CF₃, —OCHF₂, or —OCF₃; preferably, the substituted ferrocene is substituted with C₁-C₆ alkyl, -alkyl-N—(C₁-C₆ alkyl), —OH, —O—(C₁-C₆ alkyl), —NH—(C₁-C₆ alkyl); more preferably, the substituted ferrocene is substituted with C₁-C₆ alkyl, -alkyl-N—(C₁-C₆ alkyl); more preferably, the substituted ferrocene is substituted with -alkyl-N—(C₁-C₆ alkyl); most preferably, the substituted ferrocene is

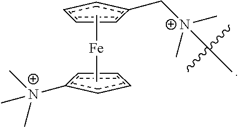

16. The compound of claim 1 having the structure:

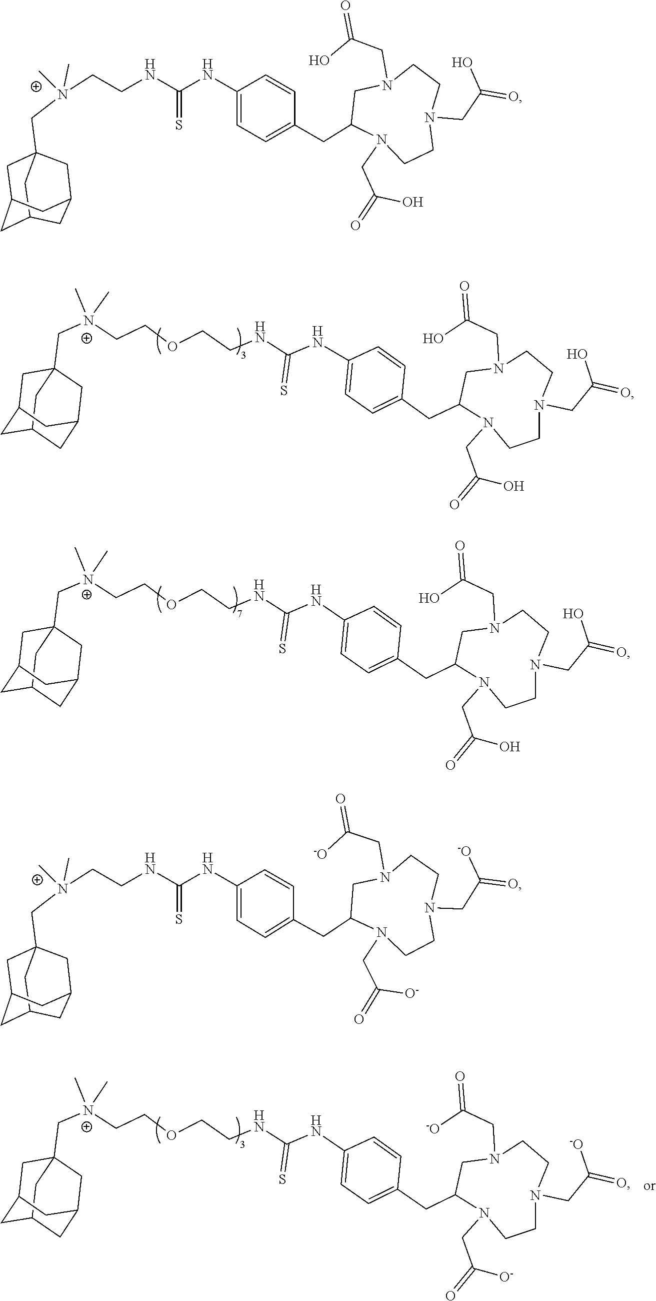

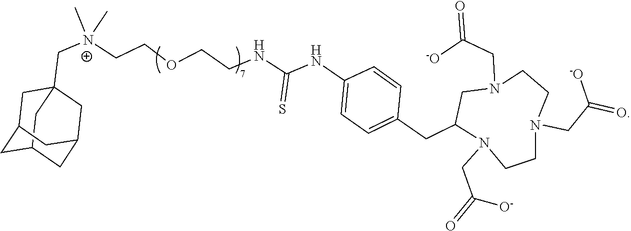

17. A metal complex comprising the compound of any one of claims 1-16, wherein the compound coordinates to a metal.

18. The metal complex of claim 17 having the structure: 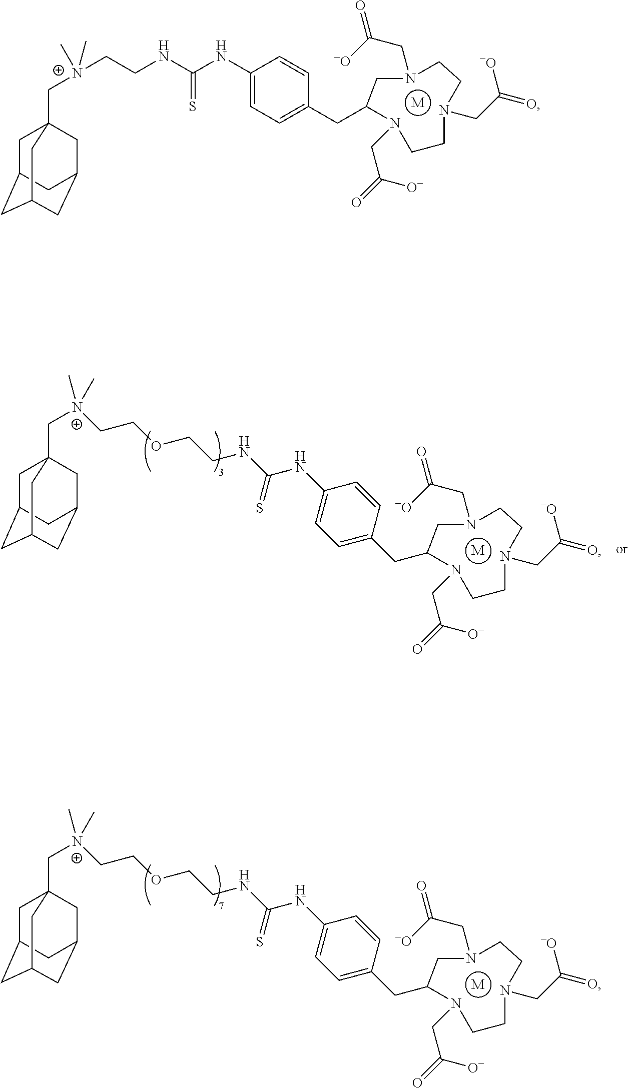
wherein \textcircled{M} is the metal.

19. The metal complex of any one of claims 17-18, wherein the metal is Copper-62 (⁶²Cu), Copper-64 (⁶⁴Cu), Copper-67 (⁶⁷Cu), Gallium-68 (⁶⁸Ga) Scandium-44 (⁴′Sc), Scandium-47 (⁴⁷Sc), Scandium-43 (⁴³Sc), Lead-203 (²⁰³Pb), Lead-212 (²¹²Pb), Lanthanum-132 (¹³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb); preferably, the metal is Copper-62 (⁶²Cu), Copper-64 (⁶⁴Cu), Copper-67 (⁶⁷Cu), Scandium-44 (⁴⁴Sc), Scandium-47 (⁴⁷Sc), or Scandium-43 (⁴³Sc); more preferably, the metal is Copper-64 (⁶⁴Cu).

20. A pharmaceutical composition comprising the metal complex of any one of claim 17-19 and a marker attached to a host molecule.

21. The pharmaceutical composition of claim 20, wherein the marker is a tumor marker or a cancer marker.

22. The pharmaceutical composition of any one of claims 20-21, wherein the tumor marker is a prostate-specific antigen (PSA), prostatic acid phosphatase (PAP), cancer antigen 125 (CA 125), carcinoembryonic antigen (CEA), alpha-fetoprotein (AFP), human chorionic gonadotropin (HCG), cancer antigen 19-9 (CA 19-9), cancer antigen 15-3 (CA 15-3), cancer antigen 27-29 (CA 27-29), lactate dehydrogenase (LDH), or neuron-specific enolase (NSE); more preferably, the tumor marker is a prostate-specific antigen (PSA), cancer antigen 125 (CA 125), carcinoembryonic antigen (CEA), cancer antigen 19-9 (CA 19-9), cancer antigen 15-3 (CA 15-3), or cancer antigen 27-29 (CA 27-29); more preferably the tumor marker is a prostate-specific antigen (PSA) or carcinoembryonic antigen (CEA); most preferably, the tumor marker is a carcinoembryonic antigen (CEA).

23. The pharmaceutical composition of any one of claims 20-22, wherein the host molecule comprises cucurbit[5]uril, cucurbit[6]uril, cucurbit[7]uril, cucurbit[8]uril, cucurbit[10]uril, cucurbit[14]uril, cyclodextrin, or calix-[5]-arenes; more preferably, the host molecule comprises cucurbit[5]uril, cucurbit[6]uril, cucurbit[7]uril, cucurbit[8]uril, or cucurbit[10]uril; more preferably, the host molecule comprises cucurbit[5]uril, cucurbit[6]uril, cucurbit[7]uril, or cucurbit[8]uril; more preferably, the host molecule comprises cucurbit[7]uril, or cucurbit[8]uril; most preferably, the host molecule is cucurbit[7]uril.

24. The pharmaceutical composition of any one of claims 20-23, wherein the interaction between the host and the guest molecule is a non-covalent interaction, preferably, the non-covalent interaction is ion-ion interaction, ion-dipole interaction, dipole-dipole interaction, hydrogen bonding, cation-7c interaction, 7-t interaction, van der Waals interaction or hydrophobic interaction; more preferably, the interaction is ion-ion interaction or van der Waals interaction.

25. The pharmaceutical composition of any one of claims 20-24, wherein the metal complex and the host molecule form a high affinity host-guest complex.

26. A method of detecting cells in a subject comprises administering an effective amount of metal complex of any one of claims 17-19 to the subject who contains a marker attached to a host molecule.

27. A method of detecting cells in a subject comprises administering an effective amount of a marker attached to a host molecule of any one of claims 20-25 to the subject who contains a guest molecule.

28. A method of detecting cells in a subject comprising administering an effective amount of the pharmaceutical composition of any one of claims 19-24 to the subject and imaging the subject with a molecular imaging device to detect the composition in the subject.

29. A method of imaging cells in a subject comprising:
1) administering to the subject an effective amount of the composition of any one of claims 19-24, wherein the composition specifically accumulates at the cells in the subject;
2) detecting in the subject the location of the composition; and
3) obtaining an image of the cells in the subject based on the location of the composition in the subject.

30. A method of detecting the presence of cells in a subject which comprises determining if an amount of the composition of any one of claims 20-25 is present in the subject at a period of time after administration of the composition to the subject, thereby detecting the presence of the cells based on the amount of the composition determined to be present in the subject.

31. The method of claim 28, wherein the metal complex and the host molecule are applied concurrently, or wherein the host molecule is applied first, and the metal complex is applied after a period of time.

32. The method of claim 31, wherein the period of time is 24 hours, 48 hours, 72, hours, 96 hours, 120 hours, or 144 hours; preferably, the period of time 72 hours.

33. The method of any one of claims 26-32, wherein the cells are cancer cells or tumor cells.

34. The method of claim 33, wherein the cancer cells or tumor cells have elevated levels of proteins or antigens, or both.

35. The method of any one of claims 33-34, wherein the cancer is lung cancer, breast cancer, prostate cancer, cervical cancer, pancreatic cancer, colon cancer, ovarian cancer, stomach cancer, esophagus cancer, skin cancer, heart cancer, liver cancer, bronchial cancer, testicular cancer, kidney cancer, bladder cancer, spleen cancer, thymus cancer, thyroid cancer, brain cancer, or gall bladder cancer.

36. The method of any one of claims 33-34, wherein the tumor is bone tumor, brain tumor, malignant soft tissue tumor, organ tumor, ovarian germ cell tumor, gland tumor, lymphatic tumor, or skin tumor.

37. The method of any one of claims 26-36, wherein the subject is a mammal, preferably, the subject is a human.

38. The method of claim 28, wherein the molecular imaging device is a PET imaging device.

39. Use of an effective amount of the metal complex of any one of claims 17-19 to detect cells within a subject, wherein the subject contains a marker attached to a host molecule.

40. Use of an effective amount of the host molecule of any one of claims 20-25 to detect cells in a subject, wherein the subject contains a guest molecule.

41. Use of an effective amount of the composition of any one of claims 20-26 to image a subject with a molecular imaging device to detect cells in a subject.

42. The present invention provides a compound having the structure: 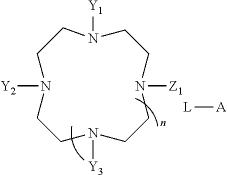
wherein
n is 0 or 1;
Y₁, Y₂ and Y₃ are each, independently, alkyl-CO₂NH₂, alkyl-N-(alkyl-CO₂R₄)₂ or alkyl-N—(CO₂R₄)₂,
wherein each occurrence of R₄ is, independently, —H, NH₂, alkyl, alkenyl, alkynyl, alkyl-aryl, alkyl-heteroaryl, aryl, heteroaryl, alkyl-CF₃ or —Si(alkyl)₃; preferably, R₄ is, independently, —H, NH₂,
Z₁ is 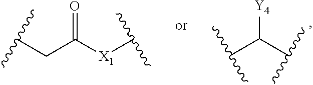
wherein X₁ is NH, O or S, and
Y₄ is —CO₂H, —CO₂R₅, aryl-CO₂H, heteroaryl-CO₂H, aryl-CO₂R₅ or heteroaryl-CO₂R₅,
wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃;
A is a targeting moiety; and
L is a chemical linker,
or a pharmaceutically acceptable salt of the compound.
